(19) United States
(12) Patent Application Publication
(10) Pub. No.: US 2017/0007608 A1
(43) Pub. Date: Jan. 12, 2017

(54) ACTIVATORS OF PYRUVATE KINASE M2 AND METHODS OF TREATING DISEASE

(71) Applicant: AGIOS PHARMACEUTICALS, INC, Cambridge, MA (US)

(72) Inventor: Charles Kung, Arlington, MA (US)

(21) Appl. No.: 15/271,075

(22) Filed: Sep. 20, 2016

Related U.S. Application Data
(63) Continuation of application No. 14/351,480, filed on Apr. 11, 2014.
(60) Provisional application No. 61/546,873, filed on Oct. 13, 2011.

(51) Int. Cl. A61K 31/5025; G01N 33/574; C12Q 1/68; A61K 31/496; A61K 45/06

(52) U.S. Cl. CPC A61K 31/5025; A61K 31/496; A61K 45/06; C12Q 1/6886; G01N 33/57484; G01N 33/57426; G01N 2333/916

(57) ABSTRACT

The invention described herein features methods, compositions, and kits that utilize activators of pyruvate kinase M2 (PKM2) for the treatment or amelioration of disorders related to PKM2 function and characterized by abnormally low levels of serine.

### CLAIM OF PRIORITY

This application is a continuation of U.S. Ser. No. 14/351,480, filed Apr. 11, 2014, which is a national stage application under 35 U.S.C. §371 of International Application No. PCT/US2012/060099, filed Oct. 12, 2012, published as International Publication No. WO2013/056153 on Apr. 18, 2013, which claims priority from U.S. Ser. No. 61/546,873, filed Oct. 13, 2011, each of which is incorporated herein by reference in its entirety.

### BACKGROUND OF THE INVENTION

Cancer cells rely primarily on glycolysis to generate cellular energy, while the majority of “normal” cells in adult tissues utilize aerobic respiration. This fundamental difference in cellular metabolism between cancer cells and normal cells, termed aerobic glycolysis or the Warburg Effect, has been exploited for diagnostic purposes, but has not yet been exploited for therapeutic benefit.

Pyruvate kinase (PK) is a metabolic enzyme that converts phosphoenolpyruvate to pyruvate during glycolysis. Four PK isoforms exist in mammals: the L and R isoforms are expressed in liver and red blood cells, respectively, the M1 isoform is expressed in most adult tissues, and the M2 isoform is a splice variant of M1 expressed during embryonic development. All tumor cells exclusively express the embryonic M2 isoform. A well-known difference between the M1 and M2 isoforms of PK is that M2 is a low-activity enzyme that relies on allosteric activation by the upstream glycolytic intermediate, fructose-1,6-bisphosphate (FBP), whereas M1 is a constitutively active enzyme.

### SUMMARY OF THE INVENTION

The invention features methods, compositions, and kits that utilize activators of pyruvate kinase M2 (PKM2) for the treatment or amelioration of a disorder or disease related to PKM2 function and where the disease or disorder, such as a proliferative disorder, is characterized by abnormally low levels of serine.

The invention also features methods, compositions, and kits that utilize activators of PKM2 for the treatment or amelioration of a disorder or disease related to PKM2 function, and where the disease or disorder, such as a proliferative disorder, is characterized by abnormally low levels of phosphoserine phosphatase mRNA or protein, or abnormally low levels of phosphoserine phosphatase activity.

The invention also features methods, compositions, and kits that utilize activators of PKM2 for the treatment or amelioration of a disorder or disease related to PKM2 function, and where the disease or disorder, such as a proliferative disorder, is characterized by a mutation, amplication or misregulation in a gene involved in serine biosynthesis (e.g., a phosphoglycerate dehydrogenase (PHGDH) gene, phosphoserine aminotransferase (PSAT) genes, or phosphoserine phosphatase (PSPH) gene).

In one aspect, the invention features a method of determining whether a patient who has a proliferative disorder, such as a cancer, is a candidate for treatment with a compound that activates PKM2, where the method includes measuring serine levels in a biological sample from the patient and determining if the serine levels are reduced as compared to a control sample. The biological sample is, for example, a serum sample, a tissue sample, as from a biopsy, e.g., from a sample a tumor sample, or from a tissue suspected of having cancerous cells. As used herein, a “control sample” is a sample from a non-diseased subject, i.e., a subject, who does not have the disorder, or from a tissue of the same type that does not have a tumor or cancerous cells. In one embodiment, the serine levels from the biological sample are compared to levels determined to be normal, as an industry standard, in a population from data compiled from a set of non-diseased samples.

In one embodiment, if the serine levels are abnormally low, then it is determined that the patient is a candidate for treatment with a compound that activates PKM2. A candidate for treatment with a compound that activates PKM2 can be predicted to experience a positive result following administration of the compound, e.g., the candidate will experience improved symptoms of the disorder. For example, tumor size in a candidate who has cancer will stop growing, or shrink, or disappear, or a metastasis will slow in its progress, or the patient will go into remission, following administration of the compound.

In one embodiments, the activator of PKM2 is selected from a compound of formula (I) or a pharmaceutically acceptable salt thereof:

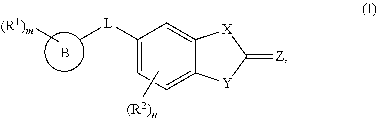

wherein:

m is an integer from 0 to 5;

each R¹ is independently selected from C₁-C₆ alkyl, C₁-C₆ alkoxy, C₁-C₆ haloalkyl, C₁₋₆ haloalkoxy, halo, acetyl, —NO₂, aryl, aralkyl, heteroaryl, —SO₂-aryl, —C(O)—NR^{b}-aryl, —C(O)— aralkyl, —C(O)—C₁₋₆ alkoxy, —NR^{b}—SO₂-aryl, wherein each aryl, aralkyl and heteroaryl group is optionally substituted with 0-3 occurrences of R^{c} and wherein two R¹ groups taken together with the carbon atoms to which they are attached form a heterocyclyl ring;

n is an integer from 1 to 3;

each R² is independently selected from C₁-C₆ alkyl and halo;

B is aryl, monocyclic heteroaryl, cycloalkyl, heterocyclyl, C₁₋₆ aralkyl, or C₁₋₆ heteroaralkyl;

L is a linker selected from —SO₂—, —SO₂NR^{a}— and —NR^{a}SO₂—;

each R^{a} is independently selected from hydrogen and C₁-C₆ alkyl;

X and Y are each independently selected from O, S, NR^{b} and CH₂, wherein at least one of X and Y is O or S;

Z is O or S;

each R^{b} is independently selected from hydrogen, C₁₋₆ aralkyl, and C₁-C₆ alkyl substituted with 0-1 occurrences of R^{c}; and

R^{c} is independently selected from C₁₋₆ alkyl, C₁₋₆ alkoxy, C₁₋₆ haloalkyl, halo, NR^{d}R^{d}, and heterocyclyl and wherein two R^{c} groups taken together with the carbon atoms to which they are attached form a heterocyclyl ring; and

R^{d} is independently selected from H and C₁₋₆ alkyl.

In one embodiment, the activator of PKM2 is a compound selected from formula (II) or a pharmaceutically acceptable salt thereof:

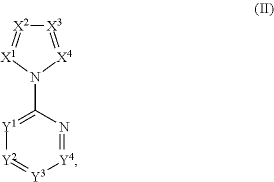

wherein

X¹ is N or CE;

X² is N or CD;

X³ is N or CB;

X⁴ is N or CA;

Y¹, Y², Y³ and Y⁴ are each independently selected from N and CR¹;

A, B, D and E are each independently selected from H, R³ and —SO₂—NR⁴R⁵;

wherein at least one of X¹, X², X³, X⁴, Y¹, Y², Y³ and Y⁴ is N; and at least one of X¹, X², X³, X⁴, is C—SO₂—NR⁴R⁵;

each R⁴ is independently selected from C₁₋₈ alkyl, aryl and heteroaryl, each of which is substituted with n occurrences of R²;

each R⁵ is independently hydrogen or C₁₋₈ alkyl;

each R¹ is independently selected from hydrogen, C₁₋₈ alkyl, C₁₋₈ terminal alkynyl, C₁₋₈ alkoxy, halogen, haloalkyl and haloalkoxy;

each R² is independently selected from halo, haloalkyl, C₁₋₄ alkyl, C₂₋₄ alkenyl, C₂₋₄ alknynyl, aryl, heteroaryl, aralkyl, heteroaralkyl, cyano, —OR^{a}, —COOR^{b} and —CONR^{c}R^{c′}; wherein two R², together with the carbons to which they are attached, may form an optionally substituted ring, each of which can be further substituted;

each R³ is independently selected from C₁₋₈ alkyl, —OR^{a}, halogen, haloalkyl, haloalkoxy and optionally substituted heteroaryl;

each R^{a} is independently selected from alkyl, haloalkyl, optionally substituted heteroaryl and optionally substituted heterocyclyl;

each R^{b} is independently alkyl; and

each R^{c} is independently selected from hydrogen and alkyl; and

n is 0, 1, 2 or 3.

In one embodiment, the activator of PKM2 is a compound selected from formula (III) or a pharmaceutically acceptable salt thereof:

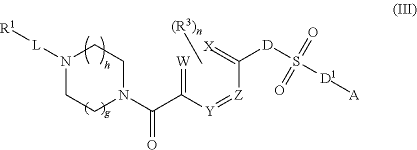

wherein:

W, X, Y and Z are each independently selected from CH or N;

D and D¹ are independently selected from a bond or NR^{b};

A is optionally substituted bicyclic heteroaryl;

L is a bond, —C(O)—, —(CR^{c}R^{c})ₘ—, —OC(O)—, —(CR^{c}R^{c})ₘ—OC(O)—, —(CR^{c}R^{c})ₘ—C(O)—, —NR^{b}C(S)—, or —NR^{b}C(O)—;

R¹ is selected from alkyl, cycloalkyl, aryl, heteroaryl, and heterocyclyl; each of which is substituted with 0-5 occurrences of R^{d};

each R³ is independently selected from halo, haloalkyl, alkyl, hydroxyl and —OR^{a} or two adjacent R³ taken together with the carbon atoms to which they are attached form an optionally substituted cyclyl;

each R^{a} is independently selected from alkyl, acyl, hydroxyalkyl and haloalkyl;

each R^{b} is independently selected from hydrogen and alkyl;

each R^{c} is independently selected from hydrogen, halo, alkyl, alkoxy and halo alkoxy or two R^{c} taken together with the carbon atoms to which they are attached form an optionally substituted cycloalkyl;

each R^{d} is independently selected from halo, haloalkyl, haloalkoxy, alkyl, alkynyl, nitro, cyano, hydroxyl, —C(O)R^{a}, —OC(O)R^{a}, —C(O)OR^{a}, —SR^{a}, —NR^{a}R^{b} and —OR^{a}, or two R^{d} taken together with the carbon atoms to which they are attached form an optionally substituted heterocyclyl;

n is 0, 1, or 2;

m is 1, 2 or 3;

h is 0, 1, 2; and

g is 0, 1 or 2.

In one embodiment, the activator of PKM2 is a compound selected from formula (IV) or a pharmaceutically acceptable salt thereof:

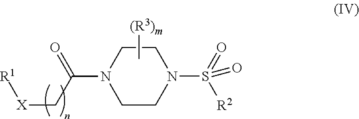

or a pharmaceutically acceptable salt thereof, wherein:

m is 0, 1 or 2;

n is 0, 1 or 2;

X is O, S, NR^{b}, alkylenyl, cycloalkylenyl, or a bond;

R¹ is selected from optionally substituted alkyl, optionally substituted aryl, optionally substituted heteroaryl, optionally substituted heterocyclyl, optionally substituted cycloalkyl, an optionally substituted aralkyl, or optionally substituted heteroaralkyl;

R² is an optionally substituted aryl or an optionally substituted heteroaryl;

each R³ is independently selected from halo, alkyl, haloalkyl and —OR^{a};

each R^{a} is independently selected from alkyl, haloalkyl and optionally substituted heteroaryl; and

each R^{b} is independently hydrogen or alkyl.

In one embodiment, the serine level in a biological sample of the patient, e.g., a tumor sample, is compared to the serine level in a control sample. A control sample may be the serum of the candidate patient, the serum of a normal patient, or cells of the same tissue as affected by the disorder, but not affected by the disorder, or cells of the same type of tissue as affected by the disorder, but from a patient who does not have the disorder.

In one embodiment, the biological sample, e.g., cells of the tumor sample, has abnormally low levels of phosphoserine phosphatase mRNA or protein, or abnormally low levels of phosphoserine phosphatase activity.

In another embodiment, cells of the biological sample have a mutation, amplication or misregulation in a gene involved in serine biosynthesis, e.g., a phosphoglycerate dehydrogenase (PHGDH) gene, phosphoserine aminotransferase (PSAT) gene, or phosphoserine phosphatase (PSPH) gene.

In another embodiment, the patient has a solid tumor, e.g., a tumor in the lung, colon or pancreas. In another embodiment, the patient has leukemia.

In one aspect, the invention features a method of monitoring the efficacy of treatment of a patient having a cancer following administration of a PKM2 activator, where the method includes monitoring serine levels in the patient following administration of the PKM2 activator. The serine levels are typically monitored at regular intervals, e.g., every one, 2, 3, 4, 5, 6, 7, days or more, or once a week or once every two or three or four weeks, or once per month, or once every two or three or four months or more, for a period of time, e.g., for 6 months or a year or longer, or for as long as the patient is receiving treatment with the PKM2 activator, such as until the patient achieves remission.

Therapeutic agents and methods of subject evaluation described herein can be combined with other therapeutic modalities, e.g., with art-known treatments.

In one aspect, the invention features a method of treating a patient with a proliferative disorder by administering a PKM2 activator and a second therapeutic agent in a serine deficient environment. In one aspect, the invention features a method of treating a patient with a proliferative disorder by administering a PKM2 activator and a second therapeutic agent that lowers the serine levels. In one embodiment, the second therapeutic agent is an inhibitor of serine metabolism. In one embodiment, the second therapeutic agent disrupts a component of the phosphoserine pathway. The second therapeutic agent, e.g., an inhibitor of serine metabolism (such as an inhibitor of phosphoglycerate dehydrogenase (PHGDH), phosphoserine aminotransferase (PSAT), or phosphoserine phosphatase (PSPH)), can be a cytotoxic agent, a serine sink, a serine biosynthesis enzyme inhibitor, a phosphoserine pathway poison. In one embodiment, the second therapeutic is a chemotherapeutic agent, such as doxorubicin, docetaxel, vinblastine, taxol (paclitaxel) and carboplatin.

In one embodiment, the second treatment (i.e., the second therapeutic agent) is, for example, surgical removal, irradiation or administration of a chemotherapeutic agent, e.g., administration of an alkylating agent. Administration (or the establishment of therapeutic levels) of the second treatment can (i) begin prior to the beginning of treatment with (or prior to the establishment of therapeutic levels of) the PKM2 activator; (ii) begin after the beginning of treatment with (or after the establishment of therapeutic levels of) the PKM2 activator; or (iii) be administered concurrently with the PKM2 activator, e.g., to achieve therapeutic levels of both, concurrently.

In one embodiment the cell proliferation-related disorder is a non-small cell lung (NSCL) tumor, and the second therapy includes administration of one or more of: an inhibitor of serine metabolism; radiation; photodynamic or laser therapy; a lobectomy or partial resection of the lung; an inhibitor of HER1/EGFR tyrosine kinase, e.g., erlotinib, e.g., Tarceva®; gemcitabine; bevacizumab (Avastin®); cetuximab (Erbitux®), Tykerb®; or Vectibix®.

In one embodiment the cell proliferation-related disorder is large cell carcinoma of the lung and the second therapy comprises one or more of: an inhibitor of serine metabolism; a lobectomy or partial resection of the lung; radiation; carboplatin; docetaxel; paclitaxel; vinorelbine; gemcitabine; cisplatin; methotrexate; mitomycin; or ifosfamide.

In one embodiment, the cell proliferation-related disorder is colon carcinoma, and the second therapy comprises one or more of: an inhibitor of serine metabolism; surgical resection of the primary and regional lymph nodes; 5-fluorouracil (5-FU); capecitabine; leucovorin; or oxaliplatin.

In one embodiment, the cell proliferation-related disorder is pancreatic carcinoma and the second therapy comprises administration of one or more of: an inhibitor of serine metabolism; radiation; surgery, e.g., a pancreaticoduodenectomy (Whipple procedure); insertion of a biliary stent; or gemcitabine.

In another embodiment, the cell proliferation-related disorder is an acute monocytic leukemia and the second therapy comprises administration of one or more of: an inhibitor of serine metabolism; radiation; a bone marrow transplant; an antibiotic; a red blood cell transfusion; transfusions of platelets; an anthracycline; all-trans retinoic acid (ATRA); arsenic trioxide

In another embodiment, the PKM2 activator is administered with a second therapeutic agent, which is an inhibitor of serine metabolism (such as an inhibitor of phosphoglycerate dehydrogenase (PHGDH), phosphoserine aminotransferase (PSAT), or phosphoserine phosphatase (PSPH)), and a third therapeutic agent, which targets the underlying medical condition, e.g., the cancer. For example, the third therapeutic agent can be a chemotherapeutic agent as described above.

In some embodiments, the methods described herein can result in reduced side effects relative to other known methods of treating cancer.

Certain tumors, or cells, characterized by abnormally low levels of serine; abnormally low levels of phosphoserine phosphatase mRNA or protein; abnormally low levels of phosphoserine phosphatase activity; or mutation, amplication or misregulation in a gene involved in serine biosynthesis pathway are sensitive to treatment with PKM2 activators, such as a compound of formulas (I)-(IV). These activators caused a decrease in cell viability when cells were cultured in an environment with low serine levels. The compound of formula (I) also inhibited tumor growth in a xenograft model.

In some embodiments the methods featured in the invention include providing a treatment to the subject wherein the treatment includes:
i) providing a PKM2 activator; and
ii) administering to the subject the PKM2 activator in a serine deficient environment,

thereby treating the subject.

In one aspect, the invention features a method of evaluating, e.g., diagnosing, a subject as having a disorder characterized by abnormally low levels of serine. The method includes analyzing a parameter related to one or more of:
a) abnormally low levels of an enzyme in the serine biosynthesis pathway, e.g., abnormally low levels of phosphoglycerate dehydrogenase (PHGDH), phosphoserine aminotransferase (PSAT), or phosphoserine phosphatase (PSPH);
b) abnormally low levels of an mRNA encoding an enzyme in the serine biosynthesis pathway, e.g., abnormally low levels of phosphoglycerate dehydrogenase (PHGDH), phosphoserine aminotransferase (PSAT), or phosphoserine phosphatase (PSPH);

or
c) a mutation, amplication or misregulation in a gene encoding an enzyme in the serine biosynthesis pathway, e.g., abnormally low levels of phosphoglycerate dehydrogenase (PHGDH), phosphoserine aminotransferase (PSAT), or phosphoserine phosphatase (PSPH);
thereby evaluating the subject.

In one embodiment, “analyzing” comprises performing a procedure, e.g., a test, to provide data or information on one or more of a-c, e.g., performing a method that results in a physical change in a sample, in the subject, or in a device or reagent used in the analysis, or which results in the formation of an image representative of the data. The sample can be a tissue sample, e.g., a tumor tissue sample, or a bodily fluid, such as a blood or serum sample, from the subject. The analysis can include an immuno analysis (e.g., immunohistochemistry or in situ analysis), an enzymatic activity assay, a branched DNA assay, a Northern analysis, or reverse transcription coupled to polymerase chain reaction.

Methods of obtaining and analyzing samples, and the in vivo analysis in subjects, described elsewhere herein, e.g., in the section entitled, “Methods of evaluating samples and/or subjects,” can be combined with this method. In another embodiment analyzing comprises receiving data or information from such test from another party. In one embodiment the analyzing includes receiving data or information from such test from another party and, the method includes, responsive to that data or information, administering a treatment to the subject.

As described herein, the evaluation can be used in a number of applications, e.g., for diagnosis, prognosis, staging, determination of treatment efficacy, patient selection, or drug selection.

Thus, in one embodiment method further comprises, e.g., following analysis of one or more of a-c above:
diagnosing the subject, e.g., diagnosing the subject as having a cell proliferation-related disorder, e.g., a disorder characterized by an abnormally low level of serine, and by unwanted cell proliferation, e.g., cancer, or a precancerous disorder;
staging the subject, e.g., determining the stage of a cell proliferation-related disorder, e.g., a disorder characterized by unwanted cell proliferation, e.g., cancer, or a precancerous disorder;
providing a prognosis for the subject, e.g., providing a prognosis for a cell proliferation-related disorder, e.g., a disorder characterized by unwanted cell proliferation, e.g., cancer, or a precancerous disorder;
determining the efficacy of a treatment, e.g., the efficacy of a PKM2 activator, alone or in combination with one or more of an inhibitor of serine metabolism, a chemotherapeutic agent, irradiation or surgery; and
selecting the subject for a treatment for a cell proliferation-related disorder, e.g., a disorder characterized by abnormally low serine levels, and by unwanted cell proliferation, e.g., cancer, or a precancerous disorder.

In one embodiment, a subject diagnosed as having a proliferative disorder associated with abnormally low levels of serine (or by abnormally low levels of phosphoserine phosphatase mRNA or protein; abnormally low levels of phosphoserine phosphatase activity; or mutation, amplication or misregulation in a gene involved in serine biosynthesis pathway) is receives a good prognosis if the subject is administered an activator of PKM2, e.g., a compound of formula (I), (II), (III), or (IV), of Table 1 or Table 2, FIG. 10A-10A-44 or 11A-11F. By “good prognosis” is meant that the subject is expected to survive longer than if the subject were not administered the PKM2 activator, or that the subject's tumor or cancer will diminish or slow in its progression, to a greater extent than if the subject were not administered the activator of PKM2.

The selection can be based on the need for amelioration of a condition associated with or resulting from abnormally low serine levels. For example, if it is determined that the subject has a cell proliferation-related disorder, e.g., cancer, or a precancerous disorder characterized by unwanted, i.e., abnormally low levels of serine, selecting the subject for treatment with a therapeutic agent described herein, e.g., a PKM2 activator (e.g., a small molecule);
correlating the analysis with an outcome or a prognosis;
providing a value for an analysis on which the evaluation is based, e.g., the value for a parameter correlated to the presence, distribution, or level of serine, or serine precursor, or enzyme involved in the serine biosynthesis pathway;
providing a recommendation for treatment of the subject; or
memorializing a result of, or output from, the method, e.g., a measurement made in the course of performing the method, and optionally transmitting the memorialization to a party, e.g., the subject, a healthcare provider, or an entity that pays for the subject's treatment, e.g., a government, insurance company, or other third party payer.

As described herein, the evaluation can provide information on which a number of decisions or treatments can be based.

Thus, in one embodiment the result of the evaluation, e.g., of a cell-proliferation disorder characterized by abnormally low levels of serine (or by abnormally low levels of phosphoserine phosphatase mRNA or protein; abnormally low levels of phosphoserine phosphatase activity; or mutation, amplication or misregulation in a gene involved in serine biosynthesis pathway), is indicative of:
the efficacy of a treatment, e.g., the efficacy of a PKM2 activator, alone or in combination with an inhibitor of serine metabolism, a chemotherapeutic agent, irradiation or surgery;

In one embodiment, relatively higher levels of PKM2 activity are indicative of responsiveness to a treatment. The result can be used as a noninvasive biomarker for clinical response. For example, evidence of elevated PKM2 activity can be predictive of better outcome in lung cancer patients (e.g., longer life expectancy).

As described herein, the evaluation can provide for the selection of a subject.

Thus, in one embodiment the method comprises, e.g., responsive to the analysis of one or more of a-c above, selecting a subject, e.g., for a treatment. The subject can be selected on a basis described herein, e.g., on the basis of:
said subject being at risk for, or having, a proliferative disorder characterized by an abnormally low level, i.e., decreased, level of serine, or a serine precursor, or an enzyme involved in the serine biosynthesis pathway;
said subject being in need of, or being able to benefit from, a therapeutic agent of a type described herein;
said subject being in need of, or being able to benefit from, a compound that activates PKM2; or
said subject being in need of, or being able to benefit from, a compound that inhibits serine biosynthesis.

In one embodiment, evaluation includes selecting the subject, e.g., for treatment with an anti-neoplastic agent, on the establishment of, or determination that, the subject has a proliferative disorder characterized by abnormally low levels of serine (abnormally low levels of phosphoserine phosphatase mRNA or protein; abnormally low levels of phosphoserine phosphatase activity; or mutation, amplication or misregulation in a gene involved in serine biosynthesis pathway).

As described herein, the evaluations provided for by methods described herein allow the selection of optimal treatment regimens.

Thus, in one embodiment the method includes, e.g., responsive to the analysis of one or more of a-c above, selecting a treatment for the subject, e.g., selecting a treatment on a basis disclosed herein. The treatment can be the administration of a therapeutic agent disclosed herein. The treatment can be selected on the basis that it is useful in treating a disorder characterized by abnormally low levels of serine (or by abnormally low levels of phosphoserine phosphatase mRNA or protein; abnormally low levels of phosphoserine phosphatase activity; or mutation, amplication or misregulation in a gene involved in serine biosynthesis pathway).

In one embodiment, evaluation includes selecting the subject, e.g., for treatment.

In some embodiments, the treatment is the administration of a therapeutic agent described herein.

The methods can also include treating a subject, e.g., with a treatment selected in response to, or on the basis of, an evaluation made in the method.

Thus, in one embodiment the method includes, e.g., responsive to the analysis of one or more of a-c above, administering a treatment to the subject, e.g., the administration of a therapeutic agent of a type described herein.

In one embodiment, which includes selecting or administering a treatment for the subject, the subject:
has not yet been treated for the cell proliferation-related disorder, and the selected or administered treatment is the initial or first line treatment;
has already been treated for the cell proliferation-related disorder, and the selected or administered treatment results in an alteration of the existing treatment;
has already been treated for the cell proliferation-related disorder, and the selected treatment results in continuation of the existing treatment; or
has already been treated for the cell proliferation-related disorder, and the selected or administered treatment is different, e.g., as compared to what was administered prior to the evaluation or to what would be administered in the presence of normal levels of serine.

In one embodiment, which includes selecting or administering a treatment for the subject, the selected or administered treatment can include:
a treatment which includes administration of a therapeutic agent at different, e.g., a greater (or lesser) dosage (e.g., different as compared to what was administered prior to the evaluation or to what would be administered in the presence of normal levels of serine);
a treatment which includes administration of a therapeutic agent at a different frequency, e.g., more or less frequently, or not at all (e.g., different as compared to what was administered prior to the evaluation or to what would be administered in the presence of normal levels of serine); or
a treatment which includes administration of a therapeutic agent in a different therapeutic setting (e.g., adding or deleting a second treatment from the treatment regimen) (e.g., different as compared to what was administered prior to the evaluation or to what would be administered in the presence of normal levels of serine).

Methods of evaluating a subject described herein can include evaluating a genotype or phenotype of a subject or biological sample. Methods of obtaining and analyzing samples, and the in vivo analysis in subjects, described elsewhere herein, e.g., in the section entitled, “Methods of evaluating samples and/or subjects,” can be combined with this method.

In one embodiment the method includes:
subjecting the subject (e.g., a subject having a proliferative disorder) to a biopsy or alternate procedure to determine the level of serine associated with the disorder;
optionally storing a parameter related to the determination, e.g., the value related to the determination, in a tangible medium; and
responsive to the determination, performing one or more of: correlating the determination with outcome or with a prognosis; providing an indication of outcome or prognosis; providing a value for an analysis on which the evaluation is based, e.g., the presence, distribution, or level of serine; providing a recommendation for treatment of the subject; selecting a course of treatment for the subject, e.g., a course of treatment described herein, e.g., selecting a course of treatment that includes an activator of PKM2; administering a course of treatment to the subject, e.g., a course of treatment described herein, e.g., a course of treatment that includes an activator of PKM2; and memorializing a result of the method or a measurement made in the course of the method, e.g., one or more of the above and/or transmitting memorialization of one or more of the above to a party, e.g., the subject, a healthcare provider, or an entity that pays for the subject's treatment, e.g., a government, insurance company, or other third party payer.

In one embodiment, the method includes confirming or determining, e.g., by direct examination or evaluation of the subject, or sample, e.g., tissue or bodily fluid (e.g., blood (e.g., blood plasma), serum, urine, lymph, or cerebrospinal fluid) therefrom (e.g., by DNA sequencing or immuno analysis, or evaluation of the presence, distribution or level of an enzyme, or mRNA encoding an enzyme, involved in the serine biosynthesis pathway), or receiving such information about the subject, e.g., that the subject has a cancer characterized by abnormally low levels of serine (or by abnormally low levels of phosphoserine phosphatase mRNA or protein; abnormally low levels of phosphoserine phosphatase activity; or mutation, amplication or misregulation in a gene involved in serine biosynthesis pathway).

In one embodiment, prior to or after treatment, the method includes evaluating the growth, size, weight, invasiveness, stage or other phenotype of the cell proliferation-related disorder.

In one embodiment the cell proliferation-related disorder is a tumor of the lung, e.g., a NSCL or a large cell carcinoma of the lung; a colon carcinoma; a pancreatic carcinoma; or an acute myeloid leukemia, e.g., an acute monocytic leukemia, and the evaluation is a, or b, or c as described above. In one embodiment the method includes evaluating a sample, e.g., a sample described herein, e.g., a tissue sample, such as a cancer sample, or a bodily fluid, e.g., serum or blood, for abnormally low levels of serine.

In one embodiment, the method includes obtaining a sample from the subject and analyzing the sample, or analyzing the subject, e.g., by evaluating the subject or the sample, e.g., by immunohistochemistry or in situ analysis, and optionally forming representations of images from the analysis, or storing the results of the analysis on a computer.

In one embodiment, the results of the analysis are compared to a reference.

In one embodiment, a value for a parameter correlated to the presence, distribution, or level, e.g., of serine, or an enzyme involved in serine biosynthesis, is determined. It can be compared with a reference value, e.g., the value for a reference subject not having abnormal presence, level, or distribution, e.g., of serine, or an enzyme involved in serine biosynthesis.

Treatment methods described herein can include evaluating a genotype or phenotype of a subject or a biological sample. Methods of obtaining and analyzing samples, and the in vivo analysis in subjects, are described elsewhere herein, such as in the section entitled, “Methods of evaluating samples and/or subjects,” can be combined with this method

In one embodiment, prior to or after treatment, the method includes evaluating the growth, size, weight, invasiveness, stage or other phenotype of the cell proliferation-related disorder.

In another embodiment, prior to or after treatment, the method includes evaluating a phenotype that is indicative of PKM2 activity. For example, levels of ADP or PEP (phosphoenolpyruvate), or production of ATP or pyruvate, can be evaluated, e.g., spectroscopically, e.g., by colorimetry or fluorometry, or by other known methods. A decrease in ADP or PEP levels, or an increase in ATP or pyruvate levels is indicative of increased PKM2 activity. In one embodiment, production of ATP is measured using luminescence by coupling the PKM2 reaction to, e.g., a luciferase reaction. An increase in lactate production is another indicator of increased PKM2 activity. In other embodiments, a decrease in any one of cellular PEP, glycerol-phosphate, ribose or deoxyribose, lipid synthesis or glucose conversion to lipid or nucleic acids or protein by the cell can be used to confirm the ability of the candidate compound to activate PKM2.

The evaluation can be by a method described herein.

In one embodiment the subject is evaluated before treatment to determine if the cell proliferation-related disorder is characterized by abnormally low levels of serine (or by abnormally low levels of phosphoserine phosphatase mRNA or protein; abnormally low levels of phosphoserine phosphatase activity; or mutation, amplication or misregulation in a gene involved in serine biosynthesis pathway). For example, evaluation of serine levels can be by assays for enzymes or substrates in the serine metabolism pathway. For example, low levels of phosphoserine phosphatase mRNA or protein in a cell can be indicative of abnormally low serine levels. mRNA levels, e.g., phosphoserine phosphatase levels can be assayed by RT-PCR, branched DNA assay, in situ hybridization or Northern blot analysis. Protein levels, e.g., levels of phosphoserine phosphatase mRNA can be assayed by immunohistochemistry, or Western blot assay using an anti-phosphoserine antibody.

Other enzymes in the serine metabolism pathway include phosphoglycerate dehydrogenase (PHGDH) and phosphoserine aminotransferase (PSAT). Low levels of either of these proteins, or mRNAs, may be indicative of low serine levels.

In one embodiment a cancer, e.g., a lung cancer (such as a non-small cell lung cancer or a large cell carcinoma of the lung), a colon carcinoma, a pancreatic carcinoma, or an acute myeloid leukemia, e.g., acute monocytic leukemia or acute promyelocytic leukemia (APL), can be analyzed, e.g., by branched DNA analysis or immunohistochemistry or Western blot analysis, before treatment, to determine if it is characterized by abnormally low serine levels.

In one embodiment, the method includes evaluating, e.g., by direct examination or evaluation of the subject, or a sample from the subject, or receiving such information about the subject, the serine level in a tissue sample, e.g., a tumor sample. As described in more detail elsewhere herein, the evaluation can be, e.g., by mRNA or protein assay, e.g., by branched DNA or immunohistochemistry, sample analysis such as serum or biopsy, or by analysis of surgical material. In some embodiments, this information is used to determine or confirm that a proliferation-related disorder, e.g., a cancer, is characterized by abnormally low serine levels.

In one embodiment, before and/or after treatment has begun, the subject is evaluated or monitored by a method described herein, e.g., the analysis of serine levels or indicators of serine levels, e.g., to select, diagnose or prognose the subject, to select a PKM2 activator or serine biosynthesis inhibitor or additional therapeutic agent, e.g., chemotherapeutic agent, or to evaluate response to the treatment or progression of disease.

In one embodiment the cell proliferation-related disorder is a tumor of the lung, e.g., a non-small cell lung carcinoma, and the evaluation is of the presence, distribution, or level of serine or enzymes or cofactors involved in the serine biosynthetic pathway, e.g., phosphoserine phosphatase.

In one embodiment, the disorder is other than a solid tumor. In one embodiment, the disorder is a tumor that, at the time of diagnosis or treatment, does not have a necrotic portion. In one embodiment the disorder is a tumor in which at least 30, 40, 50, 60, 70, 80 or 90% of the tumor cells have abnormally low levels of serine at the time of diagnosis or treatment.

In one embodiment the cell proliferation-related disorder is a cancer, e.g., a cancer described herein, characterized by abnormally low serine levels (or by abnormally low levels of phosphoserine phosphatase mRNA or protein; abnormally low levels of phosphoserine phosphatase activity; or mutation, amplication or misregulation in a gene involved in serine biosynthesis pathway).

In one embodiment the cell proliferation-related disorder is a tumor of the lung, e.g., an NSCLC, e.g., wherein the tumor is characterized by abnormally low levels of serine (or by abnormally low levels of phosphoserine phosphatase mRNA or protein; abnormally low levels of phosphoserine phosphatase activity; or mutation, amplication or misregulation in a gene involved in serine biosynthesis). In one embodiment, the tumor is characterized by abnormally low levels of serine, as compared to non-diseased cells of the same type.

In one embodiment the method includes selecting a subject having NSCLC characterized by abnormally low levels of serine (or by abnormally low levels of phosphoserine phosphatase mRNA or protein; abnormally low levels of phosphoserine phosphatase activity; or mutation, amplication or misregulation in a gene involved in serine biosynthesis pathway). In another embodiment, the method includes selecting a subject having NSCLC characterized by abnormally low levels of an enzyme involved in the serine biosynthesis pathway, e.g., phosphoserine phosphatase.

In one embodiment the cell proliferation-related disorder is a large cell carcinoma of the lung, e.g., a tumor of a large cell carcinoma of the lung, e.g., where the tumor is characterized by abnormally low levels of serine (or by abnormally low levels of phosphoserine phosphatase mRNA or protein; abnormally low levels of phosphoserine phosphatase activity; or mutation, amplication or misregulation in a gene involved in serine biosynthesis pathway). In one embodiment, the tumor is characterized by abnormally low levels of serine, as compared to non-diseased cells of the same type.

In one embodiment the method includes selecting a subject having a large cell carcinoma of the lung characterized by abnormally low levels of serine (or by abnormally low levels of phosphoserine phosphatase mRNA or protein; abnormally low levels of phosphoserine phosphatase activity; or mutation, amplication or misregulation in a gene involved in serine biosynthesis pathway). In another embodiment, the method includes selecting a subject having a large cell carcinoma of the lung characterized by abnormally low levels of an enzyme involved in the serine biosynthesis pathway, e.g., a phosphoserine phosphatase.

In one embodiment, the cell proliferation-related disorder is a colon carcinoma, e.g., a tumor of a colon carcinoma, e.g., where the tumor is characterized by abnormally low levels of serine (or by abnormally low levels of phosphoserine phosphatase mRNA or protein; abnormally low levels of phosphoserine phosphatase activity; or mutation, amplication or misregulation in a gene involved in serine biosynthesis pathway). In one embodiment, the tumor is characterized by abnormally low levels of serine, as compared to non-diseased cells of the same type.

In one embodiment the method includes selecting a subject having a colon carcinoma characterized by abnormally low levels of serine (or by abnormally low levels of phosphoserine phosphatase mRNA or protein; abnormally low levels of phosphoserine phosphatase activity; or mutation, amplication or misregulation in a gene involved in serine biosynthesis pathway). In another embodiment, the method includes selecting a subject having a colon carcinoma characterized by abnormally low levels of an enzyme involved in the serine biosynthesis pathway, e.g., phosphoserine phosphatase.

In one embodiment the cell proliferation-related disorder is a pancreatic carcinoma, e.g., a tumor of a pancreatic carcinoma, e.g., where the tumor is characterized by abnormally low levels of serine (or by abnormally low levels of phosphoserine phosphatase mRNA or protein; abnormally low levels of phosphoserine phosphatase activity; or mutation, amplication or misregulation in a gene involved in serine biosynthesis pathway). In one embodiment, the tumor is characterized by abnormally low levels of serine, as compared to non-diseased cells of the same type.

In one embodiment, the method includes selecting a subject having a pancreatic carcinoma characterized by abnormally low levels of serine (or by abnormally low levels of phosphoserine phosphatase mRNA or protein; abnormally low levels of phosphoserine phosphatase activity; or mutation, amplication or misregulation in a gene involved in serine biosynthesis pathway). In another embodiment, the method includes selecting a subject having a pancreatic carcinoma characterized by abnormally low levels of an enzyme involved in the serine biosynthesis pathway, e.g., phosphoserine phosphatase.

In one embodiment the cell proliferation-related disorder is an acute myeloid leukemia, e.g., acute monocytic leukemia (AMoL, or AML-M5), e.g., where cancer cells of the leukemia are characterized by abnormally low levels of serine (or by abnormally low levels of phosphoserine phosphatase mRNA or protein; abnormally low levels of phosphoserine phosphatase activity; or mutation, amplication or misregulation in a gene involved in serine biosynthesis pathway). In one embodiment, the cancerous cells are characterized by abnormally low levels of serine, as compared to non-diseased cells of the same type.

In one embodiment, the method includes selecting a subject having AML characterized by abnormally low levels of serine in the cancer cells (or by abnormally low levels of phosphoserine phosphatase mRNA or protein; abnormally low levels of phosphoserine phosphatase activity; or mutation, amplication or misregulation in a gene involved in serine biosynthesis pathway). In another embodiment, the method includes selecting a subject having AML characterized by abnormally low levels of an enzyme involved in the serine biosynthesis pathway, e.g., phosphoserine phosphatase.

In one aspect, the invention features a method of increasing the level of PKM2 activity and/or glycolysis (e.g., by inhibiting the endogenous ability of a cell in the patient to downregulate PKM2) in a patient with abnormally low levels of serine, e.g., in cells of a tumor, or in cells of a tissue having a tumor. The method includes the step of administering an effective amount of an activator, preferably a selective activator, of PKM2 to the patient in need thereof, thereby increasing the level of PKM2 activity and/or glycolysis in the patient. PKM2 is only expressed in growing cells such as cancer cells or fat cells in the patient; other tissues use other isoforms of PK. In some embodiments, an activator is used to maintain PKM2 in its active conformation or to constitutively activate pyruvate kinase activity in proliferating cells as a means to divert glucose metabolites into catabolic rather than anabolic processes in the patient.

In another aspect, the invention features a method of regulating cell proliferation in a patient in a serine deficient environment, e.g., with abnormally low levels of serine, with abnormally low levels of phosphoserine phosphatase mRNA or protein, with abnormally low levels of phosphoserine phosphatase activity, or with a mutation, amplication or misregulation in a gene involved in serine biosynthesis pathway, e.g., in cells of a tumor, or in cells of a tissue having a tumor. The method includes the step of administering an effective amount of an activator of PKM2 to the patient, thereby regulating cell proliferation in the patient. This method can inhibit growth of a transformed cell, e.g., a cancer cell, or generally inhibit growth in a PKM2-dependent cell that undergoes aerobic glycolysis. In another aspect, the invention features a method of treating a patient suffering from or susceptible to a disease or disorder associated with the function of PKM2. The method includes the step of administering an effective amount of an activator of PKM2 to the patient, thereby treating or ameliorating the disease or disorder in the patient. In another embodiment, the activator is provided in a pharmaceutical composition.

In another embodiment, the method includes identifying or selecting a patient who would benefit from activation of PKM2. The patient can be identified on the basis of having abnormally low levels of serine in a cell or tissue or serum sample of the patient (e.g., as opposed to merely being in need of treatment of the disorder (e.g., cancer)). In one embodiment, the selected patient is a patient suffering from or susceptible to a disorder or disease identified herein, e.g., a disorder characterized by unwanted cell growth or proliferation, e.g., cancer.

In another embodiment, the activator of PKM2 utilized in the methods and compositions featured in the invention operates by or has one or more of the following mechanisms or properties: the activator is an allosteric activator of PKM2; the activator stabilizes the binding of fructose 1,6-bisphosphate (FBP) in a binding pocket of PKM2; the activator inhibits the release of FBP from a binding pocket of PKM2; the activator is an agonist, e.g., an analog, of FBP, e.g., an agonist which binds PKM2 with a lower, about the same, or higher affinity than does FBP; the activator inhibits the dissolution of tetrameric PKM2; the activator promotes the assembly of tetrameric PKM2; the activator stabilizes the tetrameric conformation of PKM2; the activator inhibits the binding of a phosphotyrosine containing polypeptide to PKM2; the activator inhibits the ability of a phosphotyrosine containing polypeptide to induce the release of FBP from PKM2, e.g., by inducing a change in the conformation of PKM2, e.g., in the position of Lys433, thereby hindering the release of FBP; the activator binds to or changes the position of Lys433 relative to the FBP binding pocket; the activator selectively activates PKM2 over at least one other isoform of PK, e.g., the activator is selective for PKM2 over one or more of PKR, PKM1, or PKL; the activator has an affinity for PKM2 which is greater than its affinity for at least one other isoform of PK, e.g., PKR, PKM1, or PKL; the activator has an EC₅₀ of from about 100 micromolar to about 0.1 nanomolar, e.g., about 10 micromolar to about 0.1 nanomolar, about 1 micromolar to about 0.1 nanomolar, about 500 nanomolar to about 0.1 nanomolar, about 250 nanomolar to about 0.1 nanomolar, about 100 nanomolar to about 0.1 nanomolar, about 50 nanomolar to about 0.1 nanomolar, about 25 nanomolar to about 0.1 nanomolar, about 10 nanomolar to about 0.1 nanomolar, about 100 nanomolar to about 1 nanomolar, about 50 nanomolar to about 1 nanomolar, about 25 nanomolar to about 1 nanomolar, about 10 nanomolar to about 1 nanomolar; and/or the activator is provided at a dosage of 0.1 mg to about 3000 mg per day, e.g., about 1 mg to about 2400 mg, about 15 mg to about 2400 mg, about 15 mg to about 1500 mg, about 75 mg to about 1200 mg, or about 75 mg to about 600 mg per day.

In another embodiment, the activator is administered at a dosage and frequency sufficient to increase lactate production or oxidative phosphorylation.

The method may further include the step of co-administering to the patient in need thereof an additional therapeutic agent, e.g., an inhibitor of serine metabolism. The term “co-administering” as used herein means that an additional therapeutic agent may be administered together with an activator of this invention as part of a single dosage form or as separate, multiple dosage forms. Alternatively, the additional agent may be administered prior to, consecutively with, or following the administration of a PKM2 activator. In such combination therapy treatment, both the PKM2 activator and the additional therapeutic agent(s) are administered by conventional methods. The administration of a composition of this invention, having both a PKM2 activator and an additional therapeutic agent, to a patient does not preclude the separate administration of that same therapeutic agent, any other second therapeutic agent, or the same or different PKM2 activator to the patient at another time during a course of treatment.

When the treatment is for cancer, the additional therapeutic agent may be a chemotherapeutic agent. In another embodiment, the patient being treated for cancer is characterized by one or more of the following: cells in the cancer carry out aerobic glycolysis; the cancer tissue has increased glucose uptake, as compared to a control value for glucose uptake, e.g., as measured by 2-deoxyglucose uptake or uptake by a labeled glucose or glucose analog; the cancer is metastatic; the cancer is PET positive; or the cancer has increased PKM2 expression.

In another embodiment, the activator is administered at least twice. In still another embodiment, the activator is administered in sufficient amount and with sufficient frequency that therapeutic levels are maintained for at least 1, 3, 5, 7, 10, 20, 30, 60, or 180 days. In another embodiment, the treatment is pulsatile or repeated and each administration provides therapeutic levels that are maintained for at least 1, 3, 5, 7, 10, or 20 days. In some specific embodiments, the additional therapeutic agent is an inhibitor of glutamine metabolism.

In another aspect, the invention features a method of evaluating a candidate compound, e.g., a PKM2 activator, e.g., for the ability to inhibit tumor growth, or cell viability or proliferation, in an environment having abnormally low levels of serine, (or having abnormally low levels of phosphoserine phosphatase mRNA or protein; abnormally low levels of phosphoserine phosphatase activity; or a mutation, amplication or misregulation in a gene involved in serine biosynthesis pathway) e.g., for use as an anti-proliferative or anti-cancer agent.

In one embodiment, the method includes:
optionally supplying the candidate compound;
contacting the candidate compound with a biological sample, e.g., a cell in culture or a subject having a tumor; and

evaluating the ability of the candidate compound to modulate, e.g., inhibit or promote, the cell proliferation in the environment (e.g., in the culture, or in the tumor),

thereby evaluating the candidate compound.

In one embodiment, the subject is an animal model with a xenograft, or an animal having a proliferative disorder, e.g., a leukemia, characterized by abnormally low serine levels. In another embodiment, the cells in culture are from a cancer cell line, e.g., a lung cancer (e.g., NSCL or large cell lung carcinoma), pancreatic cancer, colon cancer, or leukemia. In another embodiment, the cell is a cultured cell, e.g., a primary cell, a secondary cell, or a cell line. In yet another embodiment, the cell is an A549 cell, an NCI-H460 cell, a DU-145 cell, a Colo205 cell, an LN18 cell, a MiaPaca-2 cell, or a THP-1 cell.

In one embodiment, the cell is from a subject, e.g., a subject having cancer, e.g., a cancer characterized by abnormally low levels of serine, e.g., at the tumor site, such as within the tumor or in the area surrounding the tumor.

In another embodiment, the evaluating step includes an immuno analysis, an enzymatic activity assay, a branched DNA assay, a Northern blot analysis, or reverse transcription coupled to polymerase chain reaction, such as to determine the effect of the compound on levels of phosphoglycerate dehydrogenase (PHGDH), phosphoserine aminotransferase (PSAT), or phosphoserine phosphatase (PSPH).

In one aspect, the invention provides a method of evaluating or processing a therapeutic agent, e.g., a therapeutic agent referred to herein, e.g., a therapeutic agent that results in a activation of PKM2 in a cell or tissue having abnormally low levels of serine (or having abnormally low levels of phosphoserine phosphatase mRNA or protein; abnormally low levels of phosphoserine phosphatase activity; or a mutation, amplication or misregulation in a gene involved in serine biosynthesis pathway).

The method includes:

providing, e.g., by testing a sample, a value (e.g., a test value) for a parameter related to a property of the therapeutic agent, e.g., the ability to convert PEP to pyruvate,

optionally, providing a determination of whether the value determined for the parameter meets a preselected criterion, e.g., is present, or is present within a preselected range,

thereby evaluating or processing the therapeutic agent.

In one embodiment the therapeutic agent is approved for use in humans by a government agency, e.g., the FDA.

In one embodiment, the parameter is correlated to the ability to activate PKM2, e.g., the therapeutic agent is an activator that binds to PKM2 protein and inhibits release of FBP from PKM2.

In another embodiment, the parameter is correlated to the level of PEP or pyruvate, and, e.g., the therapeutic agent is an activator, which reduces the level of PEP or increases the amount of pyruvate.

In one embodiment, the method includes providing a comparison of the value determined for a parameter with a reference value or values, to thereby evaluate the therapeutic agent. In another embodiment, the comparison includes determining if a test value determined for the therapeutic agent has a preselected relationship with the reference value, e.g., determining if it meets the reference value. The value need not be a numerical value but, e.g., can be merely an indication of whether an activity is present.

In one embodiment, the method includes determining if a test value is equal to or greater than a reference value, if it is less than or equal to a reference value, or if it falls within a range (either inclusive or exclusive of one or both endpoints). In one embodiment, the test value, or an indication of whether the preselected criterion is met, can be memorialized, e.g., in a computer readable record.

In another embodiment, a decision or step is taken, e.g., a sample containing the therapeutic agent, or a batch of the therapeutic agent, is classified, selected, accepted or discarded, released or withheld, processed into a drug product, shipped, moved to a different location, formulated, labeled, packaged, contacted with, or put into, a container, e.g., a gas or liquid tight container, released into commerce, or sold or offered for sale, or a record made or altered to reflect the determination, depending on whether the preselected criterion is met. For example, based on the result of the determination or whether an activity is present, or upon comparison to a reference standard, the batch from which the sample is taken can be processed, e.g., as just described.

The evaluation of the presence or level of activity can show if the therapeutic agent meets a reference standard.

In one embodiment, methods and compositions disclosed herein are useful from a process standpoint, e.g., to monitor or ensure batch-to-batch consistency or quality, or to evaluate a sample with regard to a reference, e.g., a preselected value.

In one embodiment, the method can be used to determine if a test batch of a therapeutic agent can be expected to have one or more of the properties. Such properties can include a property listed on the product insert of a therapeutic agent, a property appearing in a compendium, e.g., the U.S. Pharmacopea, or a property required by a regulatory agency, e.g., the FDA, for commercial use.

In one embodiment, the method includes testing the therapeutic agent for its effect on the wildtype activity of a PKM2 protein, and providing a determination of whether the value determined meets a preselected criterion, e.g., is present, or is present within a preselected range.

In one embodiment the method includes:
contacting a therapeutic agent that is an activator of PKM2,
determining a value related to the activation of PKM2, e.g., conversion of PEP to pyruvate, and
comparing the value determined with a reference value, e.g., a range of values, for the activation of PKM2, e.g., conversion of PEP to pyruvate. In one embodiment the reference value is an FDA required value, e.g., a release criteria.

In one aspect, the invention features a method of evaluating a sample of a therapeutic agent, e.g., a therapeutic agent referred to herein, that includes receiving data with regard to an activity of the therapeutic agent; providing a record which includes said data and optionally includes an identifier for a batch of therapeutic agent; submitting said record to a decision-maker, e.g., a government agency, e.g., the FDA; optionally, receiving a communication from said decision maker; optionally, deciding whether to release market the batch of therapeutic agents based on the communication from the decision maker. In one embodiment, the method further includes releasing, or otherwise processing, e.g., as described herein, the sample.

In another aspect, the invention features a method of selecting a payment class for treatment with a therapeutic agent described herein, e.g., an activator of PKM2, for a subject having a cell proliferation-related disorder characterized by abnormally low serine levels (or by abnormally low levels of phosphoserine phosphatase mRNA or protein; abnormally low levels of phosphoserine phosphatase activity; or mutation, amplication or misregulation in a gene involved in serine biosynthesis pathway). The method includes:
providing (e.g., receiving) an evaluation of whether the subject is positive for a cell proliferation disorder associated with abnormally low levels of serine, and
performing at least one of (1) if the subject is positive selecting a first payment class, and (2) if the subject is a not positive selecting a second payment class.

In one embodiment the selection is memorialized, e.g., in a medical records system.

In another embodiment the method includes requesting the evaluation.

In another embodiment the evaluation is performed on the subject by a method described herein.

In another embodiment, the method includes communicating the selection to another party, e.g., by computer, compact disc, telephone, facsimile, email, or letter.

In another embodiment, the method includes making or authorizing payment for said treatment.

In one embodiment, payment is by a first party to a second party. In some embodiments, the first party is other than the subject. In some embodiments, the first party is selected from a third party payor, an insurance company, employer, employer sponsored health plan, HMO, or governmental entity. In some embodiments, the second party is selected from the subject, a healthcare provider, a treating physician, an HMO, a hospital, a governmental entity, or an entity which sells or supplies the drug. In some embodiments, the first party is an insurance company and the second party is selected from the subject, a healthcare provider, a treating physician, an HMO, a hospital, a governmental entity, or an entity which sells or supplies the drug. In some embodiments, the first party is a governmental entity and the second party is selected from the subject, a healthcare provider, a treating physician, an HMO, a hospital, an insurance company, or an entity which sells or supplies the drug.

The PKM2 activators of this invention may be administered in the form of a pharmaceutical composition comprising an activator of PKM2 activity and a pharmaceutically acceptable carrier. The activator is present in an amount that, when administered to a patient, is sufficient to treat a disease in a patient. The composition may be formulated as, e.g., a pill, a powder, a granulate, a suspension, an emulsion, a solution, a gel, a paste, an ointment, a cream, a foam, a lotion, a plaster, a suppository, an enema, an injectable, an implant, a spray, or an aerosol. The composition may be, e.g., formulated for targeted delivery or for extended or delayed release. The composition may be, e.g., formulated for oral, buccal, topical, rectal, subcutaneous, vaginal, inhalation, ophthalmic, parenteral, intravenous, or intramuscular administration. In some embodiments, the pharmaceutical composition further comprises an additional therapeutic agent useful in the treatment of a patient suffering from or susceptible to a disease or condition selected from cancer. In another embodiment, the additional therapeutic agent is selected from a chemotherapeutic agent. An activator of PKM2 can be administered with a chemotherapeutic agent and/or also with an inhibitor of serine metabolism.

The invention described herein features a kit that includes a pharmaceutical composition containing a PKM2 activator and instructions for administering the composition to a patient having a disease associated with abnormally low serine levels (or with by abnormally low levels of phosphoserine phosphatase mRNA or protein; abnormally low levels of phosphoserine phosphatase activity; or a mutation, amplication or misregulation in a gene involved in serine biosynthesis pathway). In some embodiments, the kit further includes at least one additional therapeutic agent. The additional therapeutic agent can be an inhibitor of serine metabolism, or an agent that is appropriate for the disease or condition to be treated by the kit, and may be selected, e.g., from any of the additional therapeutic agents set forth above for combination therapies.

By “activator” is meant an agent that increases the level of activity of PKM2 from the state of inactive monomeric or dimeric form or maintains or increases the activity of active tetrameric form of PKM2 (e.g., in the presence of an endogenous inhibitor). Increasing activity can include reducing endogenous down-regulation of PKM2 by an endogenous inhibitor (e.g., an endogenous phosphotyrosine peptide or protein). The binding of phosphotyrosine-containing peptide with activated PKM2 results in dissociation of FBP and inactivation of PKM2. Autonomous growth signaling in proliferating cells or stimulation of fat cells by insulin leads to tyrosine phosphorylation cascades. An activator can exert its effect in a number of ways including one or more of the following: an activator can render PKM2 resistant to inhibition by an inhibitor, e.g., an endogenous inhibitor; an activator inhibits release of an activator, more specifically FBP; an activator can bind to PKM2 and prevent an endogenous inhibitor from promoting the release of an endogenous activator, more specifically FBP; or an activator can inhibit the dissolution or promote the reassembly of the subunits which make up PKM2, e.g., an activator can inhibit oxidation of sulfhydryl moieties on such subunits, e.g., inhibit the oxidation of cysteine residues.

An activator can cause PKM2 activity to increase to a level that is greater than PKM2's levels (e.g., basal levels) of activity (e.g., levels seen in the absence of an endogenous or natural activator/ligand, e.g., FBP). For example, the activator may mimic the effect caused by an endogenous or natural ligand or activator (e.g., FBP). The activating effect caused by the agent may be to the same, to a greater, or to a lesser extent than the activating effect caused by an endogenous or natural ligand or activator, but the same type of effect can be caused. Peptides, nucleic acids, and small molecules may be activators. In preferred embodiments, the activator has a molecular weight in the range of 100 or 200 to 10,000, 100 or 200 to 5,000, 100 or 200 to 2,000, or more preferably 100 to 300, 200 to 500, 150 to 500, 200 to 500, 300 to 500, or 150 to 800 Daltons. Direct activators are activators which interact directly (e.g., bind) by forming a non-covalent bond such as a hydrogen, ionic, electrostatic, or hydrophobic bond, or induce a change in conformation in PKM2, including the tetrameric PKM2 molecule or the monomeric and dimeric molecules, or another activator thereof. In preferred embodiments, the direct activator forms a non-covalent bond with a specific moiety on the PKM2 or endogenous activor (e.g., FBP). Direct activators are preferred.

An expressional activator increases the expression of the PKM2 isoform at the nucleic acid level. This includes activators which induce the expression of PKM2 at the DNA level (e.g., by acting as a co-factor to induce transcription of PKM2) or the RNA level. An agent can be evaluated to determine if it is an activator by measuring either directly or indirectly the activity of the PKM2 when subjected to the agent. The activity of the agent can be measured, for example, against a control substance. In some instances, direct activation of PKM2 is measured. The activity of PKM2 can be measured, for example, by monitoring the concentration of a substrate or a product directly or indirectly.

All tumor cells exclusively express the embryonic M2 isoform of pyruvate kinase. PKM2 can serve as a target in cancer therapy. PKM2 is also expressed in adipose tissue and activated T-cells and thus activators of PKM2 can be used to treat disorders that are dependent on such cells. While not wishing to be bound by theory, it is believed that PKM2-dependent cells, e.g., cancer cells, must regulate PKM2, activating it when the cell's need for completion of glycolysis and maximal ATP production is relatively greater and inhibiting it when the cell's need for anabolic processes (growth) is relatively greater. Thus, the endogenous ability to modulate the activity of PKM2 is critically important to the cell. Therapeutic or exogenous modulation of PKM2 by inhibition or activation, e.g., constitutive activation or inhibition, defeats the endogenous modulation or regulation by the cell. Activators can be used to treat disorders related to PKM2 metabolism, e.g., disorders characterized by unwanted cell proliferation, e.g., cancer, obesity, diabetes, atherosclerosis, restenosis, and autoimmune conditions. Selective activators are preferred. Thus, activating PKM2 can mean depriving or compromising the ability of a cell to inactivate PKM2. An activator can reduce the cell's ability to down regulate PKM2 and can, for example, turn regulated PKM2 activity into constitutive PKM2 activity.

As used herein “abnormally low” levels of a compound or an enzyme refers to levels that are 5%, 10%, 20%, 30%, 40%, 50%, 60%, 70% or more lower than levels from the same person prior to having a disorder (e.g., a cancer), or as compared to levels considered to be normal for the general population who does not have the disorder, or as compared to levels in a non-cancerous tissue. For example, as used herein “abnormally low levels of serine” refers to or serine levels that are 5%, 10%, 20%, 30%, 40%, 50%, 60%, 70% or more lower than the serine levels from the same person prior to having a disorder (e.g., a cancer), or as compared to levels considered to be normal for the general population who does not have the disorder, or as compared to levels in a non-cancerous tissue. Abnormally low levels of serine can be correlated or associated with abnormally low levels of an enzyme in the serine biosynthesis pathway, or an mRNA encoding such an enzyme. For example, abnormally low levels of serine can be correlated and associated with abnormally low levels of phosphoglycerate dehydrogenase (PHGDH), phosphoserine aminotransferase (PSAT), or phosphoserine phosphatase (PSPH).

By “administering” is meant a method of giving a dosage of a pharmaceutical composition to a patient. The compositions described herein can be administered by a route selected from, e.g., ocular, inhalation, parenteral, dermal, transdermal, buccal, rectal, vaginal, sublingual, periungual, nasal, topical administration, and oral administration. Parenteral administration includes intravenous, intraperitoneal, subcutaneous, and intramuscular administration. The preferred method of administration can vary depending on various factors, e.g., the components of the composition being administered and the severity of the condition being treated.

By “chemotherapeutic agent” is meant a chemical that may be used to destroy a cancer cell, or to slow, arrest, or reverse the growth of a cancer cell. Exemplary chemotherapeutic agents include, e.g., L-asparaginase, bleomycin, busulfan carmustine (BCNU), carboplatin, chlorambucil, cladribine (2-CdA), CPTl 1 (irinotecan), cyclophosphamide, cytarabine (Ara-C), dacarbazine, daunorubicin, dexamethasone, doxorubicin (adriamycin), etoposide, fludarabine, 5-fluorouracil (5FU), hydroxyurea, idarubicin, ifosfamide, interferon-α (native or recombinant), levamisole, lomustine (CCNU), mechlorethamine (nitrogen mustard), melphalan, mercaptopurine, methotrexate, mitomycin, mitoxantrone, paclitaxel, pentostatin, prednisone, procarbazine, tamoxifen, taxol or taxol-related compounds, 6-thiogaunine, topotecan, vinblastine, vincristine, cisplatinum, carboplatinum, oxaliplatinum, or pemetrexed.

As used herein, a “cell proliferation-related disorder,” is a disorder characterized by unwanted cell proliferation or by a predisposition to lead to unwanted cell proliferation (sometimes referred to as a precancerous disorder). Examples of disorders characterized by unwanted cell proliferation include cancers, e.g., characterized by solid tumors, e.g., of the lung, such as non-small cell lung cancer and large cell carcinoma of the lung; or of the colon, pancreas. Other examples include hematological cancers, e.g., a leukemia, e.g., an acute myeloid leukemia, such as acute monocytic leukemia. Examples of disorders characterized by a predisposition to lead to unwanted cell proliferation include myelodysplasia or myelodysplastic syndrome, which are a diverse collection of hematological conditions marked by ineffective production (or dysplasia) of myeloid blood cells and risk of transformation to AML.

By “effective amount” is meant the amount of a pharmaceutical composition featured in the invention required to treat a patient suffering from or susceptible to a disease, such as, e.g., cancer, diabetes, obesity, autoimmune diseases, atherosclerosis, restenosis, and proliferation-dependent diseases. The effective amount of a pharmaceutical composition varies depending upon the manner of administration and the age, body weight, and general health of the subject. Ultimately, the attending prescriber will decide the appropriate amount and dosage regimen. Such an amount is referred to as the “effective amount.”

By “inhibitor” is meant an agent that measurably slows, stops, decreases, or inactivates the enzymatic activity of an enzyme to a level that is less than the basal level of activity of the same enzyme. By “patient” is meant any animal, e.g., mammal (e.g., a human). By “pharmaceutical composition” is meant any composition that contains at least one therapeutically or biologically active agent and is suitable for administration to a patient. For the purposes of this invention, pharmaceutical compositions suitable for delivering a therapeutic can include, e.g., eye drops, tablets, gelcaps, capsules, pills, powders, granulates, suspensions, emulsions, solutions, gels, hydrogels, oral gels, pastes, ointments, creams, plasters, drenches, delivery devices, suppositories, enemas, injectables, implants, sprays, or aerosols. Any of these formulations can be prepared by well-known and accepted methods of art. See, for example, Remington: The Science and Practice of Pharmacy (21^{st} ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

Agents useful in the pharmaceutical compositions featured in the invention may include those described herein in any of their pharmaceutically acceptable forms, including isomers such as diastereomers and enantiomers, salts, solvates, prodrugs, and polymorphs, thereof, as well as racemic mixtures of the agents described herein.

By “prodrug” is meant a molecule that, upon metabolism in the body of a subject, is chemically converted to another molecule serving a therapeutic or other pharmaceutical purpose (e.g., a drug molecule containing a carboxylic acid contains an amide or an ester bond in its prodrug form, which is cleaved upon metabolism).

By “selective” is meant at least 20%, 50%, 75%, 2-fold, 3-fold, 4-fold, 5-fold, 6-fold, or 10-fold greater inhibition of a PKM2 over a second kinase, e.g., a second pyruvate kinase, e.g., a different isoform. Thus, in some embodiments, the agent is selective for PKM2 over another isoform. For example, an agent is selective for PKM2 relative to PKM1. Selective regulation, e.g., activation, or selective modulation, are used interchangeably with specific regulation or specific modulation.

By “serine deficient environment” is meant an microenvironment that has lower levels of in serine as compared to normal circumstances, for example, a non-diseased state. A serine deficient microenvironment may be created by rapid consumption of serine. By “therapeutic agent” is meant any agent that produces a preventative, healing, curative, stabilizing, or ameliorative effect.

By “treating” is meant administering a pharmaceutical composition for prophylactic (preventative) and/or therapeutic purposes. Prophylactic treatment may be administered, for example, to a subject who is not yet ill, but who is susceptible to, or otherwise at risk of, a particular disorder, e.g., cancer. Therapeutic treatment may be administered, for example, to a subject already suffering from a disorder in order to improve or stabilize the subject's condition. Thus, in the claims and embodiments described herein, treating is the administration to a subject either for therapeutic or prophylactic purposes. In some instances, as compared with an equivalent untreated control, treatment may ameliorate a disorder or a symptom thereof by, e.g., 5%, 10%, 20%, 30%, 40%, 50%, 60%, 70%, 80%, 90%, 95%, or 100% as measured by any standard technique. In some instances, treating can result in the inhibition of a disease, the healing of an existing disease, and the amelioration of a disease.

As used herein, the term “activate” can refer to different levels of activation.

Other features and advantages of the invention will be apparent from the following detailed description, the drawings, and the claims.

### BRIEF DESCRIPTION OF THE DRAWINGS

FIG. 1 is a panel of graphs depicting the effect of an PKM2 activator AGI-752 on viability of A549 cells grown in standard media conditions under normoxia or hypoxia, as evaluated by CTP (ATP levels) or cell count.

FIGS. 2A-2D are graphs showing the effect of an PKM2 activator AGI-752 on the viability of A549 cells under various serum/glutamine concentrations.

FIG. 3 is a graph depicting the effect of an PKM2 activator AGI-752 on tumor volume in a A549 xenograft animal model.

FIG. 4 is a graph depicting the effect of an PKM2 activator AGI-752 on the growth of A549 cells in BME media.

FIGS. 5A and 5B are graphs depicting the effect of different amino acids on the growth of A549 cells in various media conditions when in the presence or absence of an PKM2 activator AGI-752

FIGS. 6A and 6B are graphs depicting the effect of serine on H460 cells grown in the presence of an PKM2 activator AGI-752.

FIGS. 7A and 7B are graphs depicting the effect of different amino acids on A549 cell growth in BME when in the presence of an PKM2 activator AGI-752.

FIGS. 8A and 8B are graphs depicting the effect of serine on H460 cells grown in BME in the presence of an PKM2 activator AGI-752.

FIGS. 9A-9F are graphs depicting the effect of various cytotoxic agents on A549 cell growth in BME.

FIGS. 10A-10A-44 represent tables of exemplary compounds and the corresponding activity of the compound. As shown in FIGS. 10A-10A-44, “A” refers to an activator of PKM2 with an EC₅₀<100 nM. “B” refers to an activator of PKM2 with an EC₅₀ between 100 nM and 500 nM. “C” refers to an activator of PKM2 with an EC₅₀ between 500 nM and 1000 nM. “D” refers to an activator of PKM2 with an EC₅₀ between 1 μM and 10 μM. “E” refers to data that is not available.

FIGS. 11A-11F represent a tables of exemplary compounds and the corresponding activity of the compound. As shown in FIGS. 11A-11F, “A” refers to an activator of PKM2 with an EC₅₀<10 μM. “B” refers to an activator of PKM2 with an EC₅₀ between 10 μM and 100 μM. “C” refers to an activator of PKM2 with an EC₅₀ greater than 100 μM.

### DETAILED DESCRIPTION

The invention described herein features methods, compositions, and kits that use of activators of PKM2 for the treatment, prevention, or amelioration of diseases related to pyruvate kinase function, including disorders characterized by unwanted cell growth or proliferation, such as cancer.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

Methods of Evaluating Samples and/or Subjects.

This section provides methods of obtaining and analyzing samples and of analyzing subjects.

Embodiments of the method include evaluation of one or more parameters related to PKM2 activation. The evaluation can be performed, e.g., to select, diagnose or prognose the subject, to select a therapeutic agent, e.g., an inhibitor, or to evaluate response to the treatment or progression of disease. In one embodiment, the evaluation, which can be performed before and/or after treatment has begun, is based, at least in part, on analysis of a tumor sample, cancer cell sample, or precancerous cell sample, from the subject. For example, a sample from the patient can be analyzed for the presence or level of serine by evaluating a parameter correlated to the presence or level of serine, or a serine precursor. Serine, or a compound or polypeptide in the serine biosynthetic pathway, can be determined by a chromatographic method, e.g., by LC-MS analysis. It can also be determined by contact with a specific binding agent, e.g., an antibody, which binds the serine or related compound, and allows detection. In one embodiment the sample is analyzed for PKM2 activity.

In one embodiment, the sample is removed from the patient and analyzed. In another embodiment, the evaluation can include one or more of performing the analysis of the sample, requesting analysis of the sample, requesting results from analysis of the sample, or receiving the results from analysis of the sample. Generally, analysis can include one or both of performing the underlying method or receiving data from another who has performed the underlying method.

In one embodiment the evaluation, which can be performed before and/or after treatment has begun, is based, at least in part, on analysis of a tissue (e.g., a tissue other than a tumor sample), or bodily fluid, or bodily product. Exemplary tissues include lymph node, skin, hair follicles and nails. Exemplary bodily fluids include blood, plasma, urine, lymph, tears, sweat, saliva, semen, and cerebrospinal fluid. Exemplary bodily products include exhaled breath. For example, the tissue, fluid or product can be analyzed for the presence or level of PKM2 activity by evaluating a parameter correlated to the presence or level of the activity. The activity in the sample can be determined by a chromatographic method, e.g., by LC-MS analysis. In one embodiment the tissue, fluid or product is removed from the patient and analyzed. In one embodiment the evaluation can include one or more of performing the analysis of the tissue, fluid or product, requesting analysis of the tissue, fluid or product, requesting results from analysis of the tissue, fluid or product, or receiving the results from analysis of the tissue, fluid or product.

A patient can be selected on the basis that the patient has a cancer characterized as having abnormally low serine levels, and then a PKM2 activator is administered to the patient. A pharmaceutical agent (e.g., a drug) for treating a subject suffering from cancer, for example, a cancer characterized as having abnormally low serine levels, can also be selected. These methods include evaluating a subject suffering from cancer to determine whether the cancer is characterized as having abnormally low serine levels, and if the cancer is characterized as having abnormally low serine levels, selecting a PKM2 activator to treat the subject. Exemplary methods of determining whether a cancer is characterized as having abnormally low serine levels are provided herein.

### Indications

Proliferating cells and fat cells express PKM2 specifically. Thus, the activators and methods used herein are particularly useful for treating disorders having unwanted activity or numbers of such cells. The invention provides optimized and selective treatments of diseases characterized by abnormally low serine levels and associated with PKM2 function. Such diseases include, for example, cancer, atherosclerosis, restenosis, obesity, autoimmune conditions, proliferation-dependent diseases, and other diseases associated with the function of PKM2.

PKM2 traps its allosteric activator, FBP, in a binding pocket bracketed by Lys433 and that collision with a Tyr-phosphorylated polypeptide is required for release of FBP from PKM2 and subsequent inhibition of enzymatic activity.

Constitutive activation of pyruvate kinase activities in cancer cells support tumorigenesis, as evidenced by replacing PKM2 activity with PKM1 in cancer cells. Note that PKM1 is constitutively active and does not bind to FBP. Together, these results show that an on-and-off switch of glycolysis by allosterically modulating the activity of PKM2 with FBP and phosphotyrosine-containing peptide(s)/proteins is required for growth of proliferating cells (e.g., cancer cells). Constitutive activation of PKM2 presents an approach to reprogram glycolysis/metabolism of proliferating cells and ameliorating diseases associated or dependent on modulation of cell glycolysis by PKM2

Diagnosis and Treatment of Diseases Associated with PKM2 Function.

Diseases treated by the methods, compositions, and kits described herein may be caused by or associated with, e.g., the function PKM2. These diseases may include disorders characterized by unwanted cell growth or proliferation, such as cancer, obesity, diabetes, atherosclerosis, restenosis, and autoimmune diseases. In particular, the disease suitable for treatment with the PKM2 activator is characterized by abnormally low levels of serine.

Cancer.

Activators of PKM2 described herein may be used in the treatment of, e.g., a cancer, and in particular, a cancer characterized as being associated with abnormally low levels of serine. For example, a tumor of the cancer can be characterized as having a abnormally low level of serine. The cancer can be, for example, a cancer of the lung, the colon or the pancreas, or the cancer can be a leukemia.

Cancers include, without limitation, leukemias (e.g., acute leukemia, acute lymphocytic leukemia, acute myelocytic leukemia, acute myeloblasts leukemia, acute promyelocyte leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma).

Proliferative diseases include, e.g., cancer (e.g., benign and malignant), benign prostatic hyperplasia, psoriasis, abnormal keratinization, lymphoproliferative disorders (e.g., a disorder in which there is abnormal proliferation of cells of the lymphatic system), chronic rheumatoid arthritis, arteriosclerosis, restenosis, and diabetic retinopathy. Proliferative diseases are described in U.S. Pat. Nos. 5,639,600 and 7,087,648, hereby incorporated by reference.

Therapy.

Therapy according to the methods described herein may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the severity of the patient's disease, and how the patient responds to the treatment.

### Compounds

Described herein are compounds and compositions that modulate PKM2, for example, activate PKM2. Compounds that activate PKM2 can be used in the treatment or amelioration of a disorder or disease related to PKM2 function, and where the disease or disorder, such as a proliferative disorder, is characterized by abnormally low levels or serine.

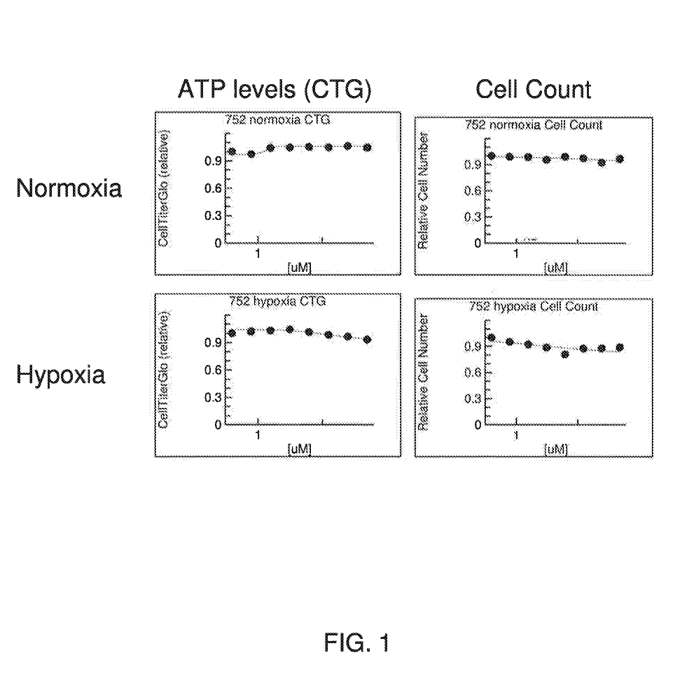

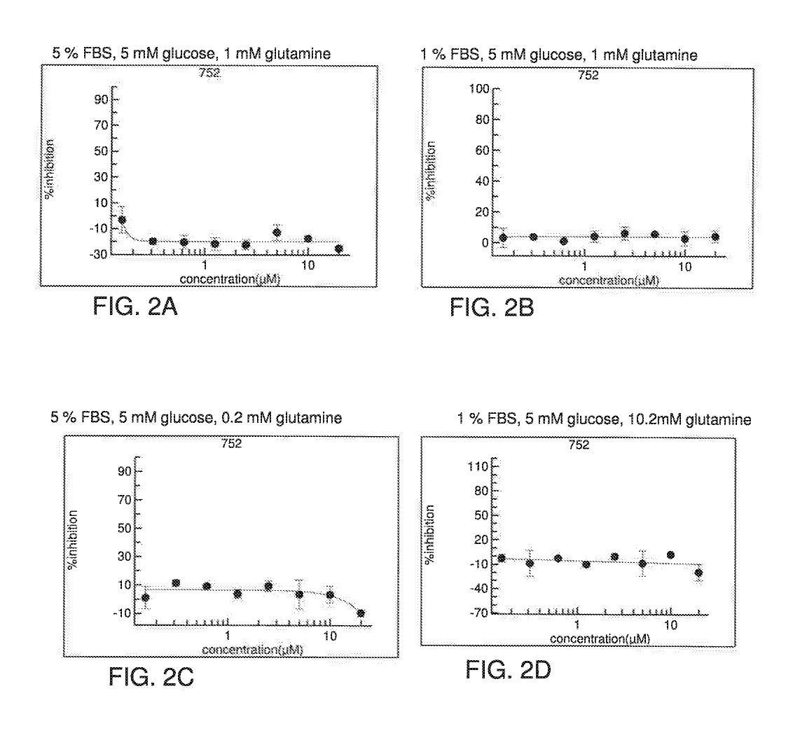

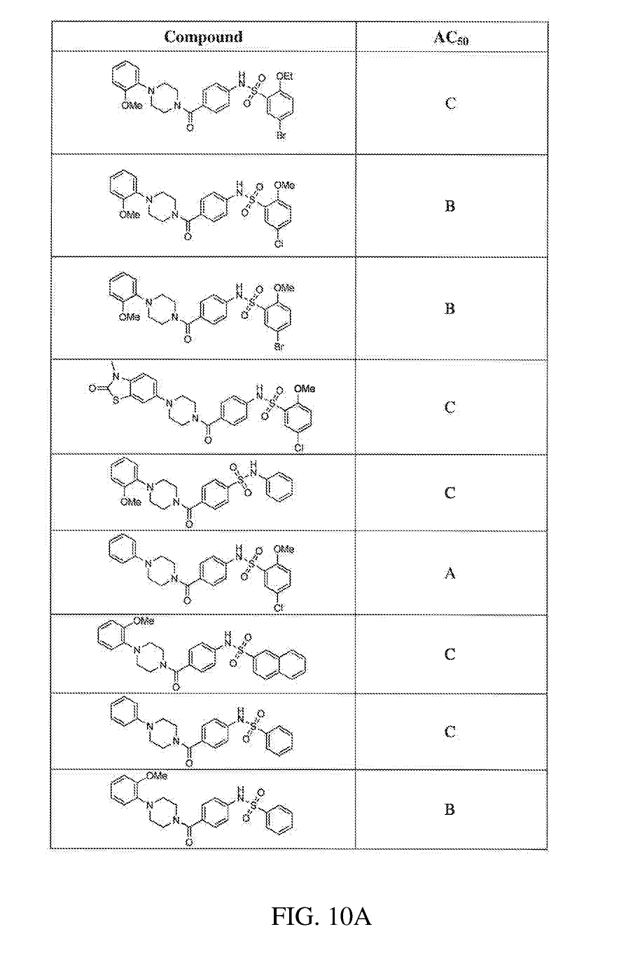

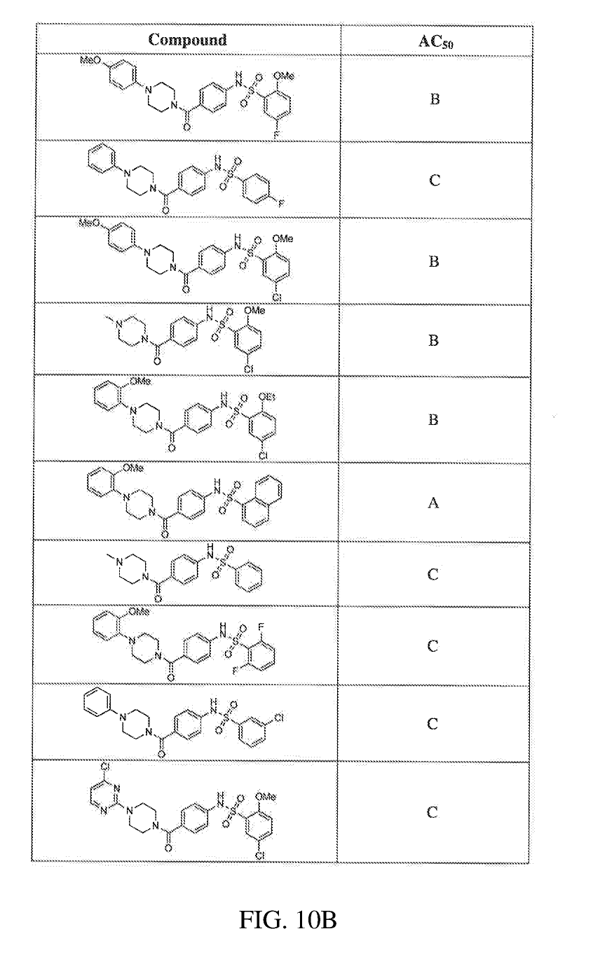

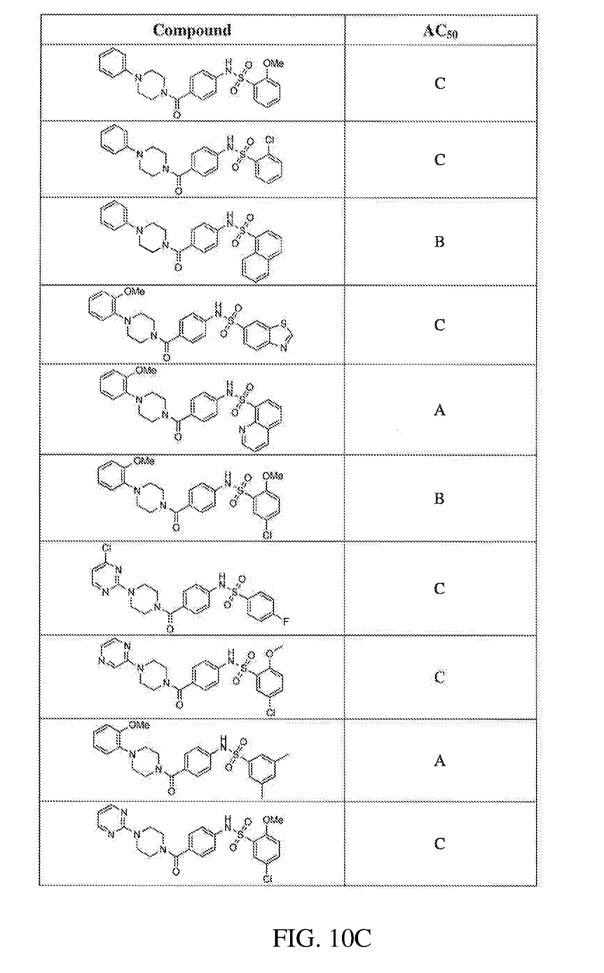

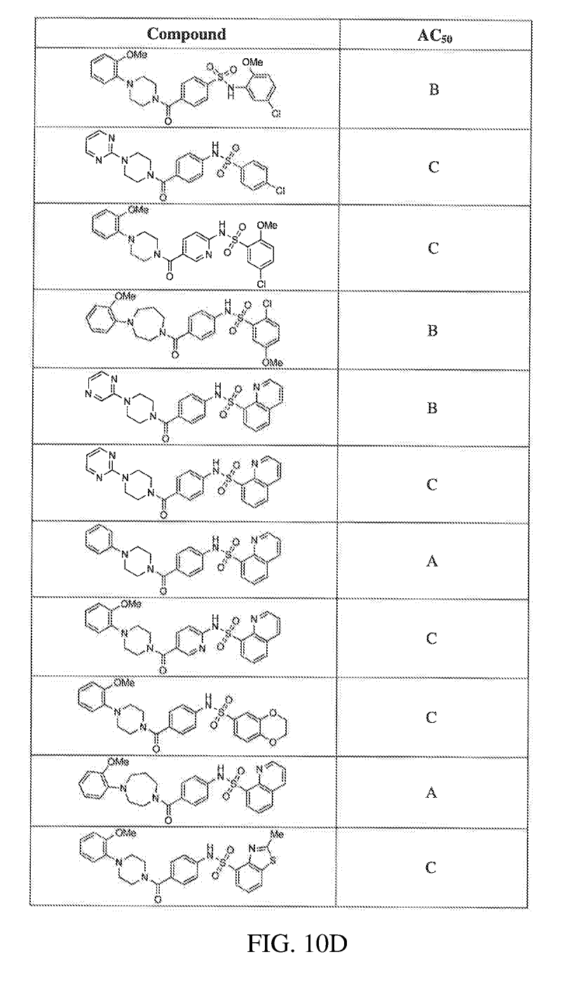

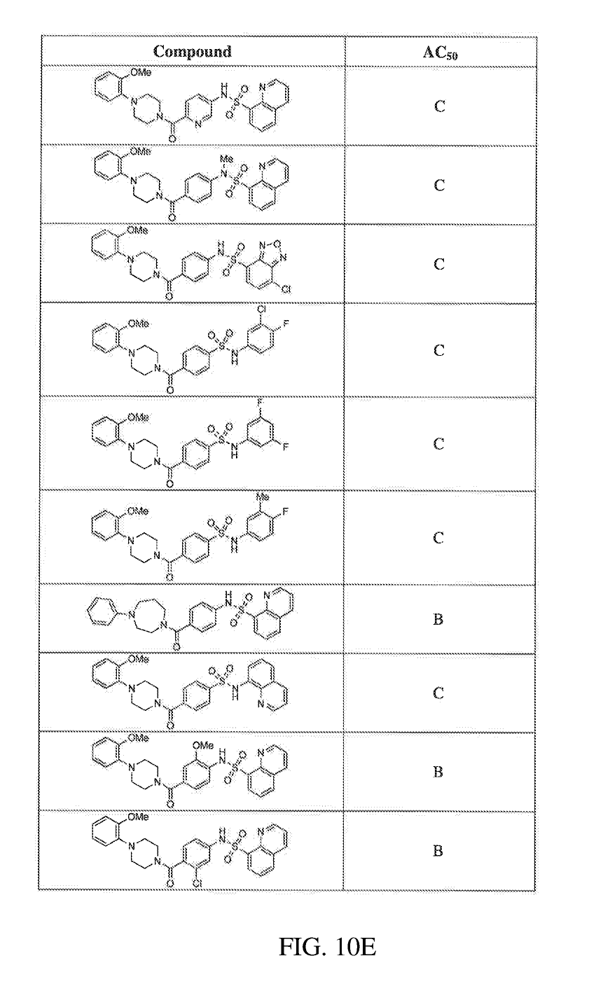

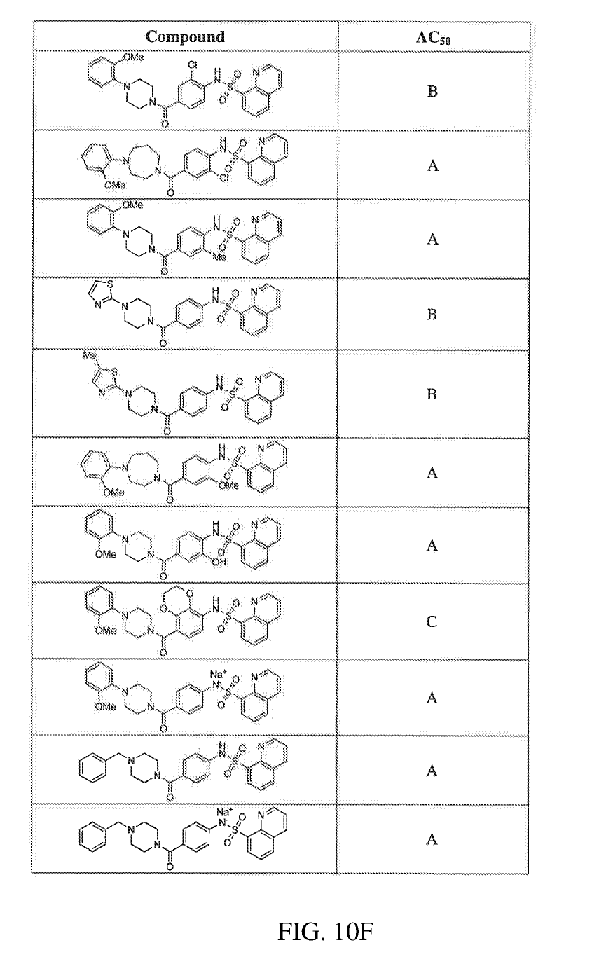

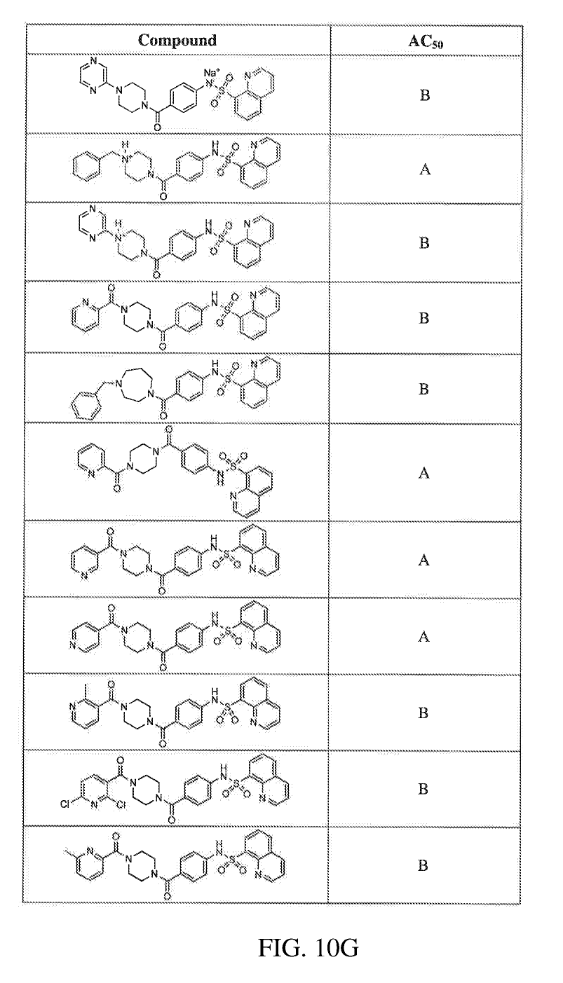

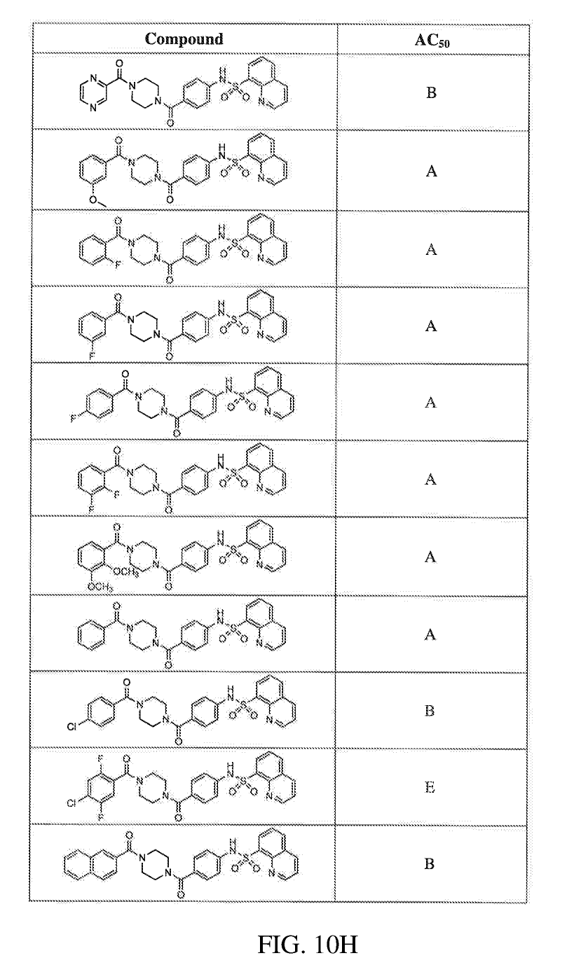

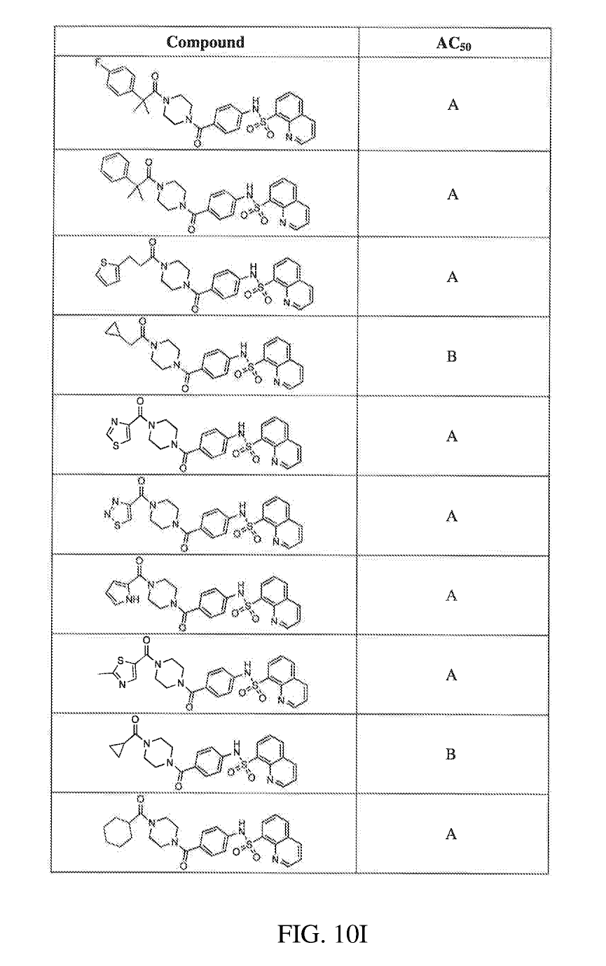

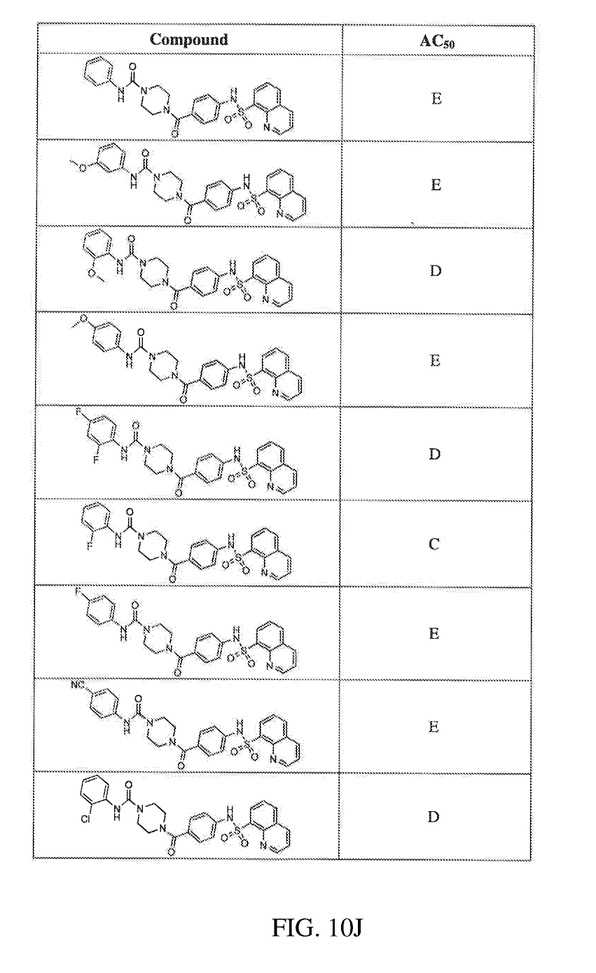

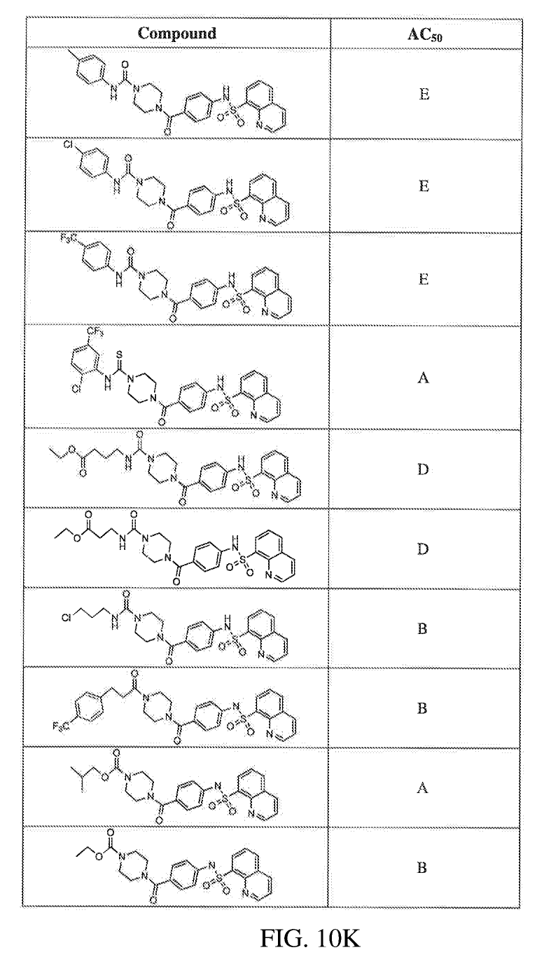

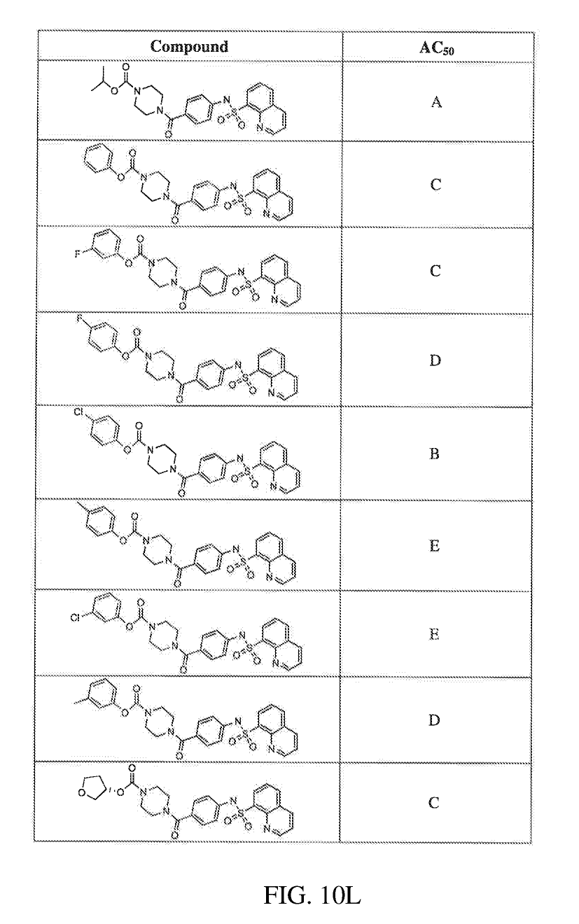

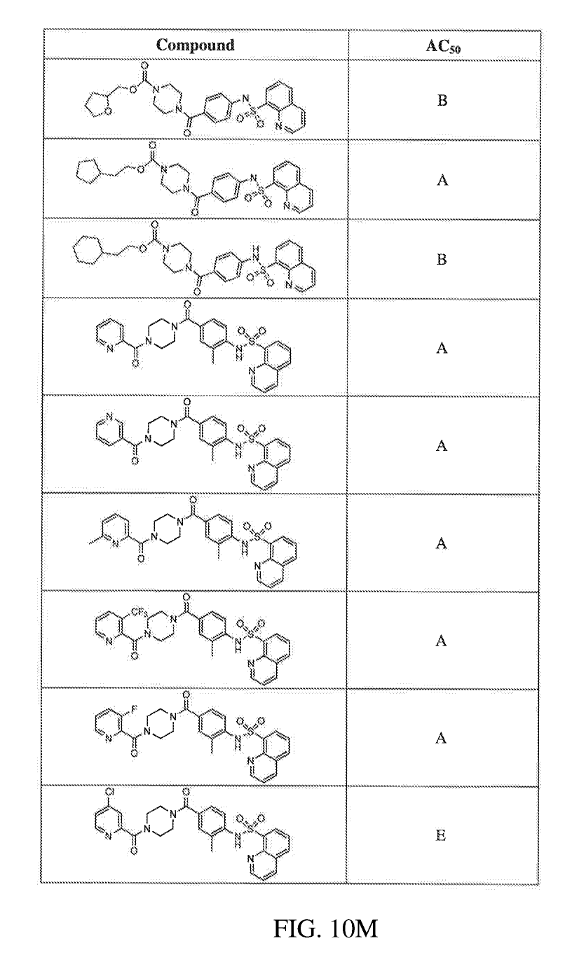

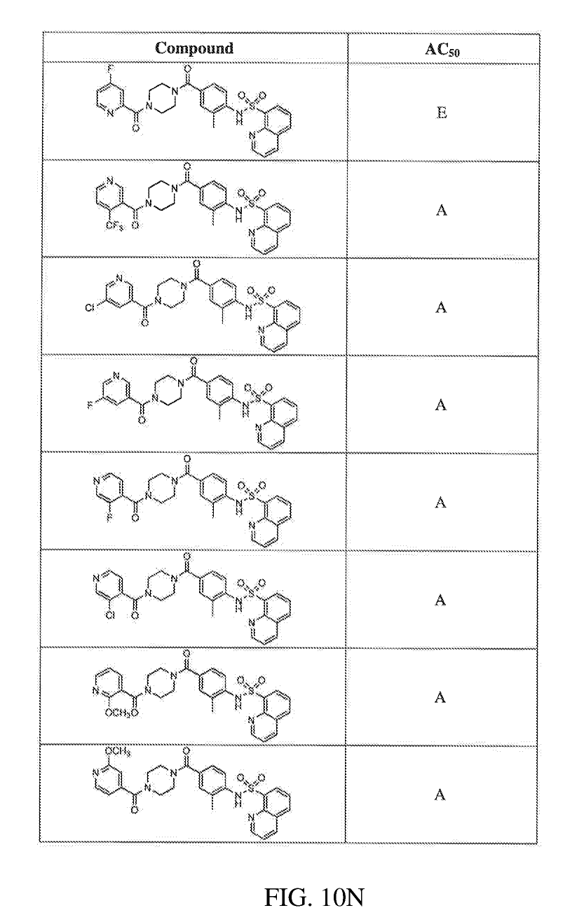

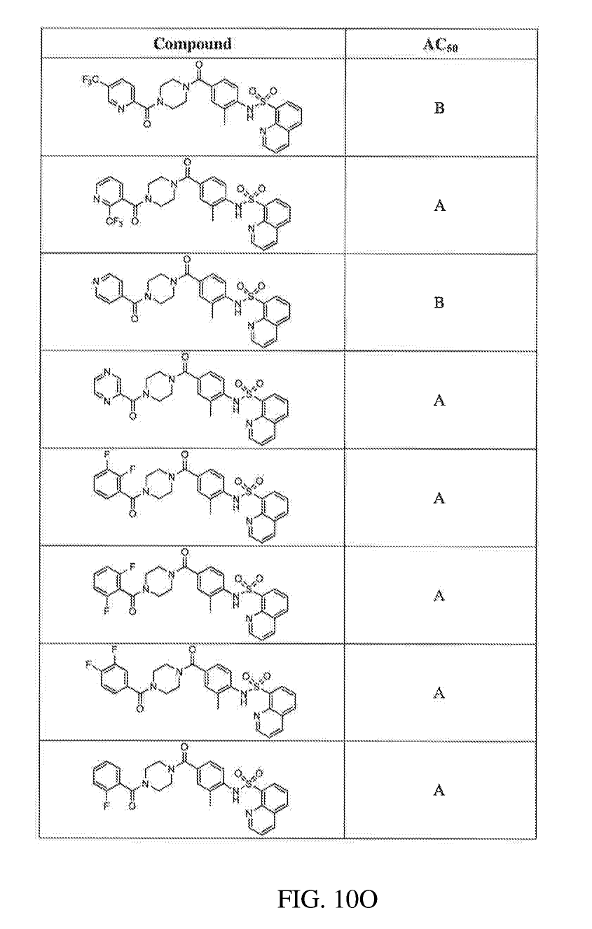

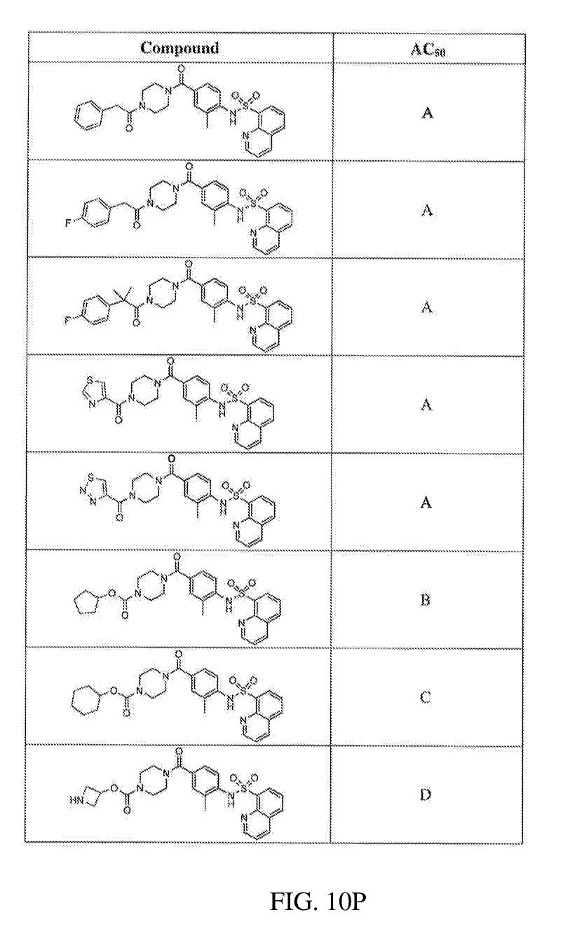

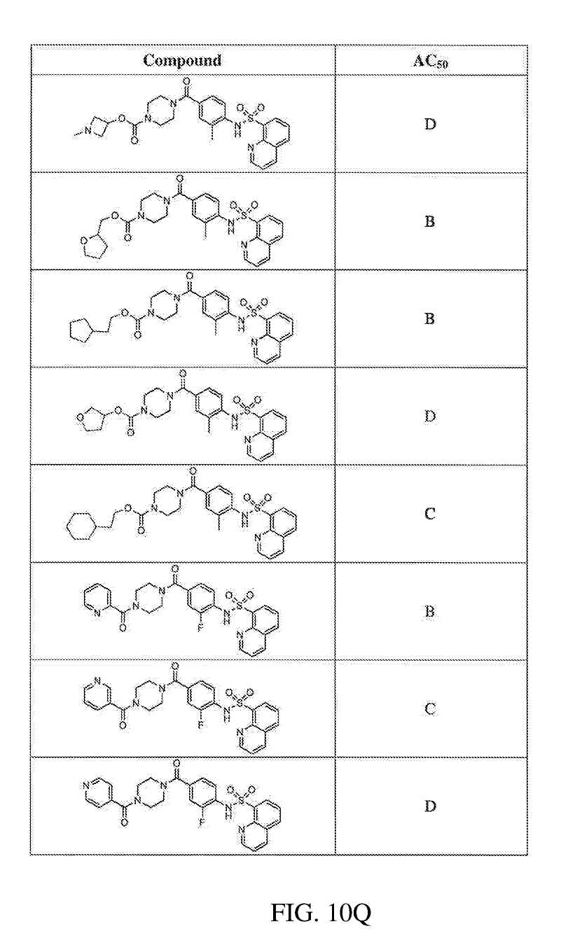

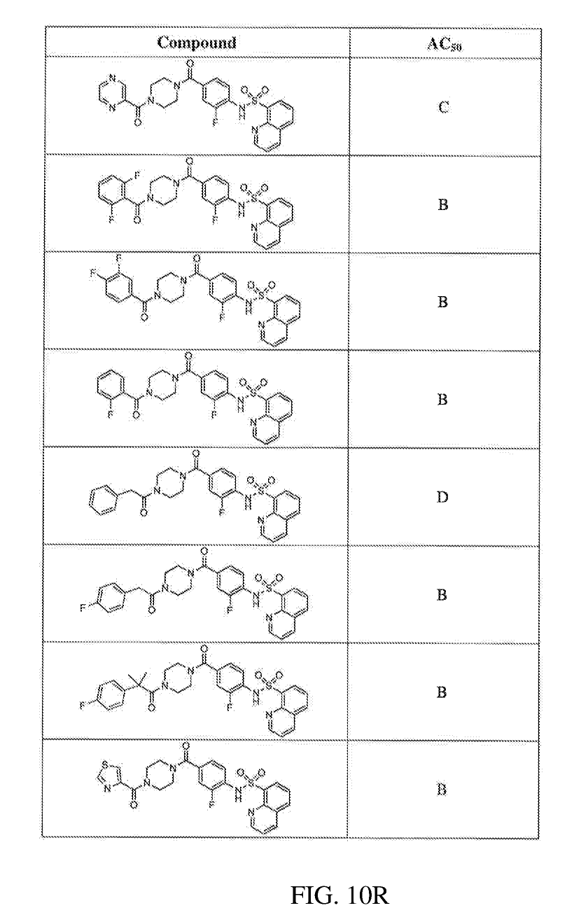

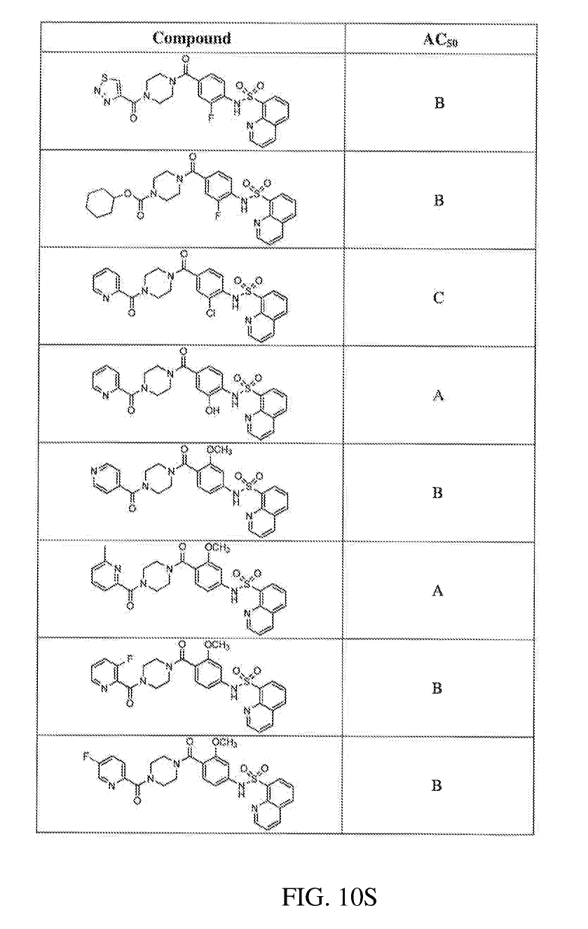

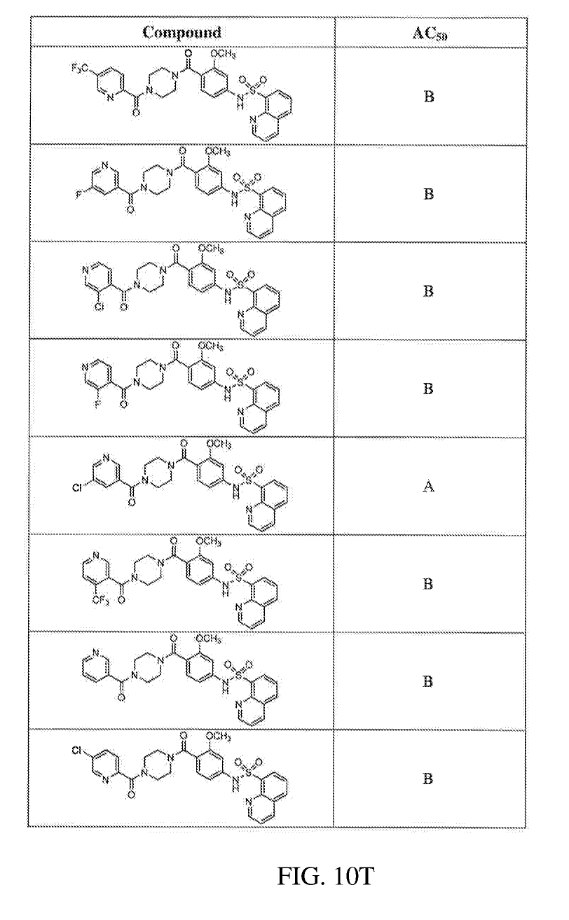

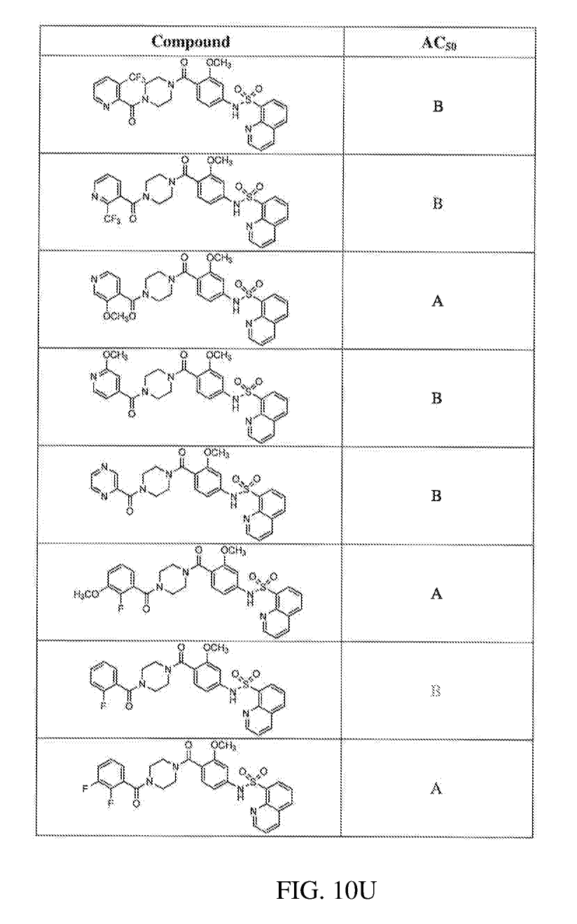

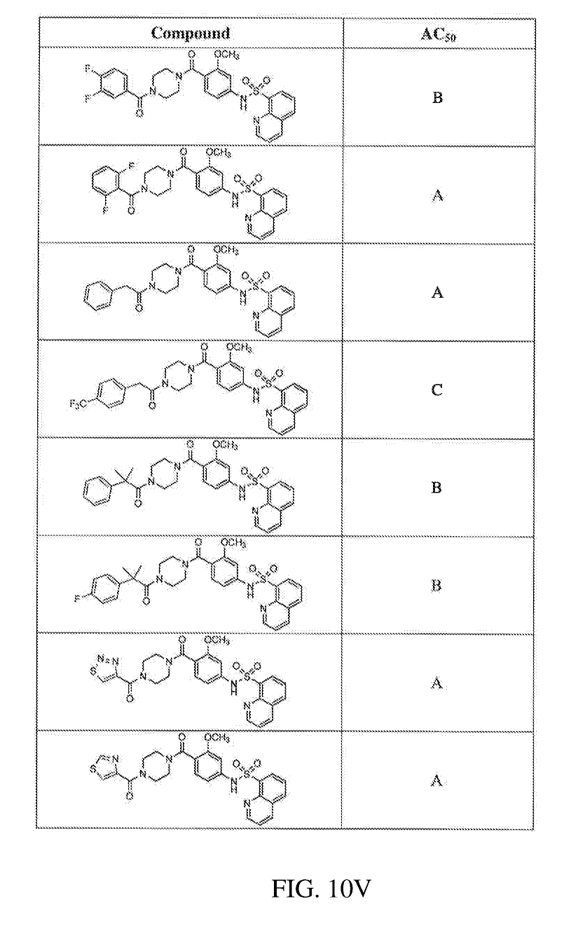

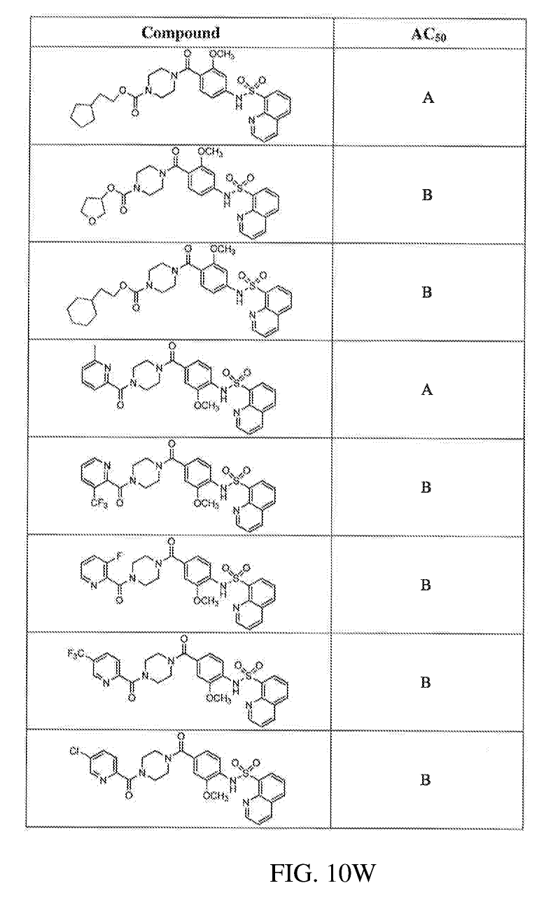

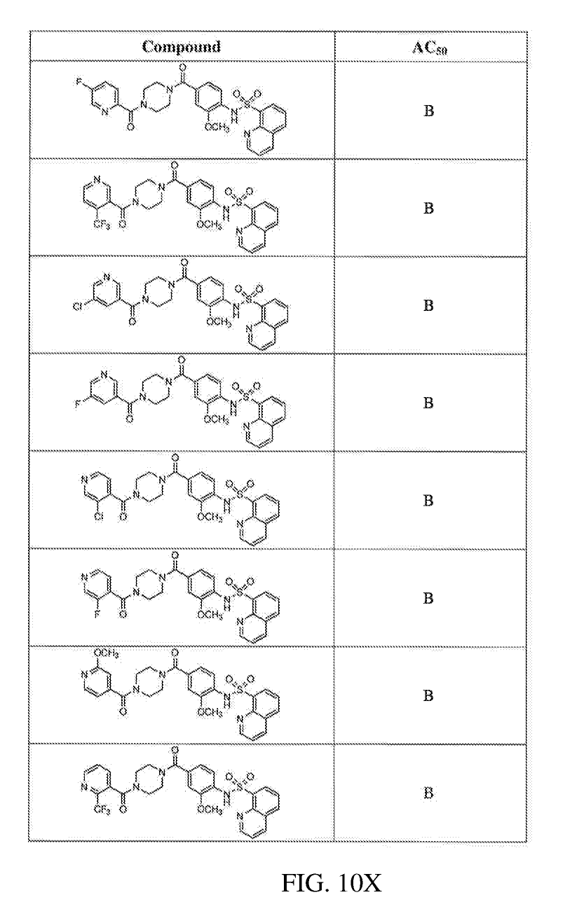

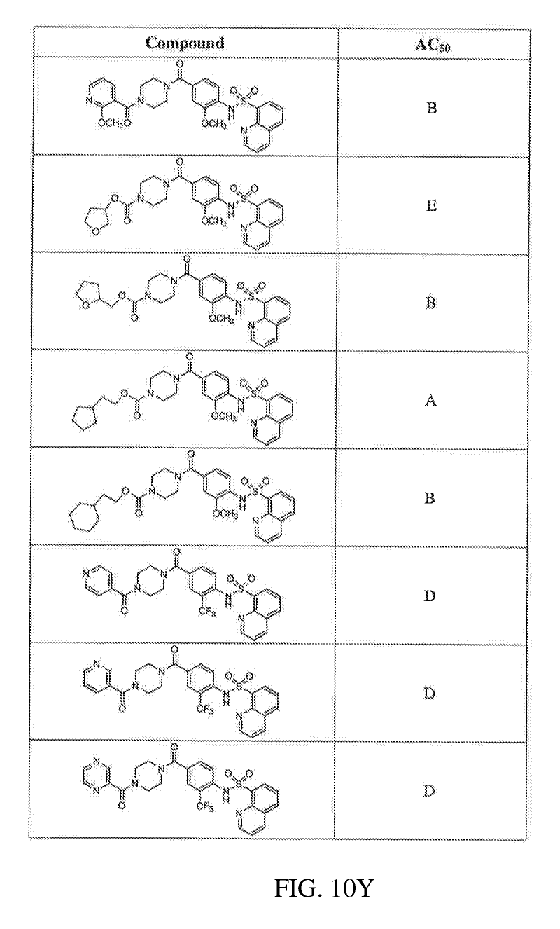

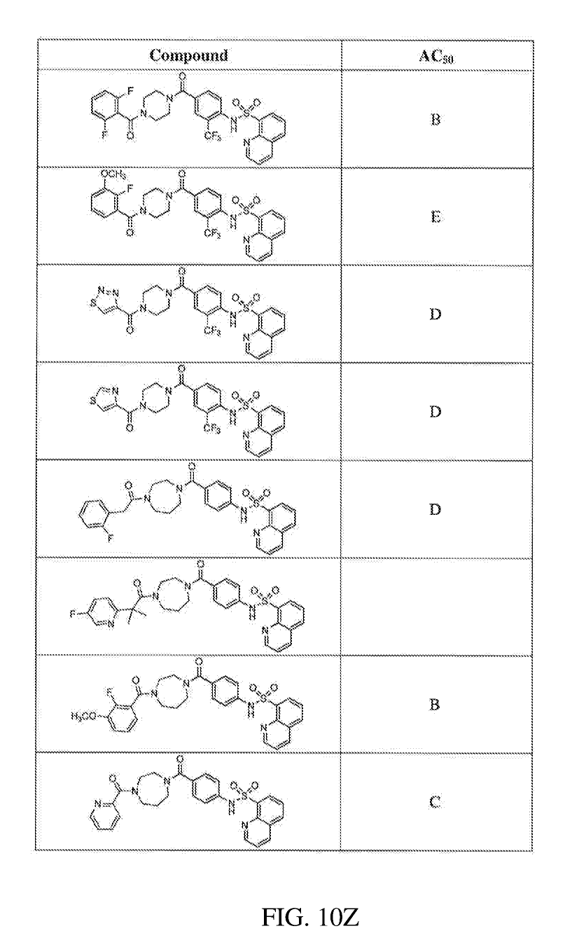

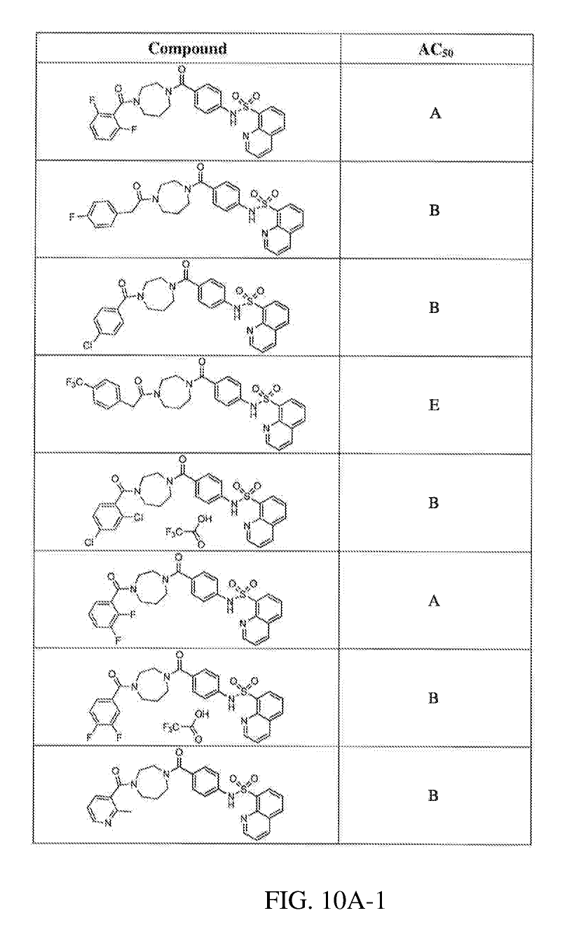

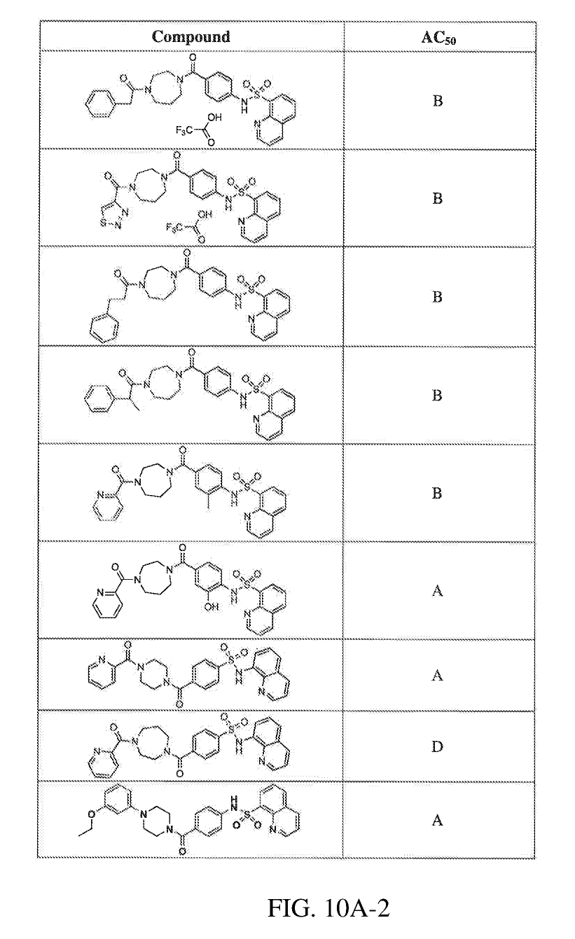

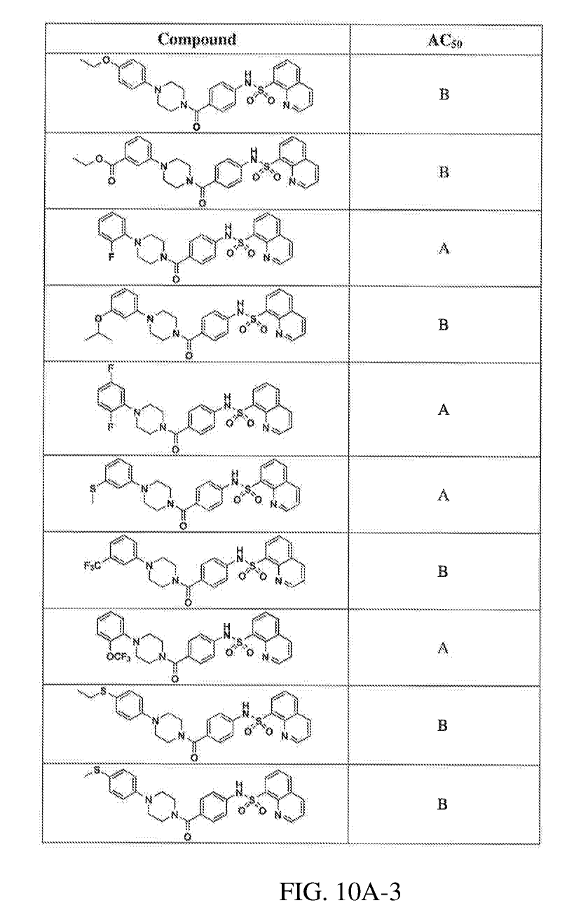

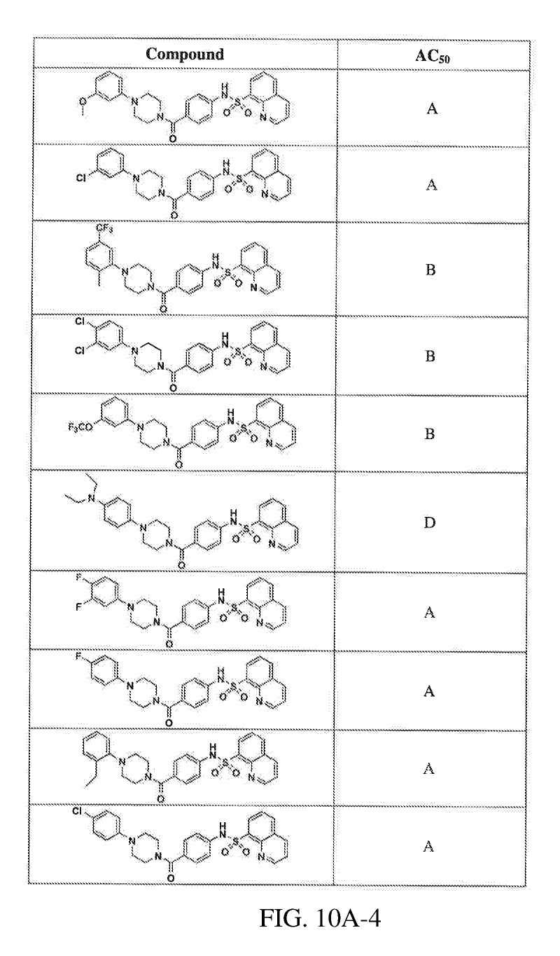

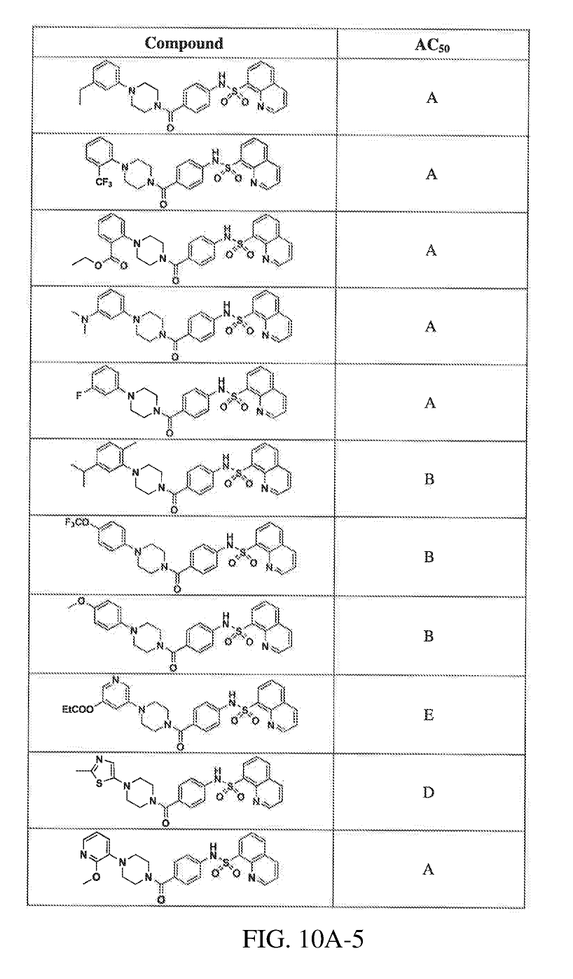

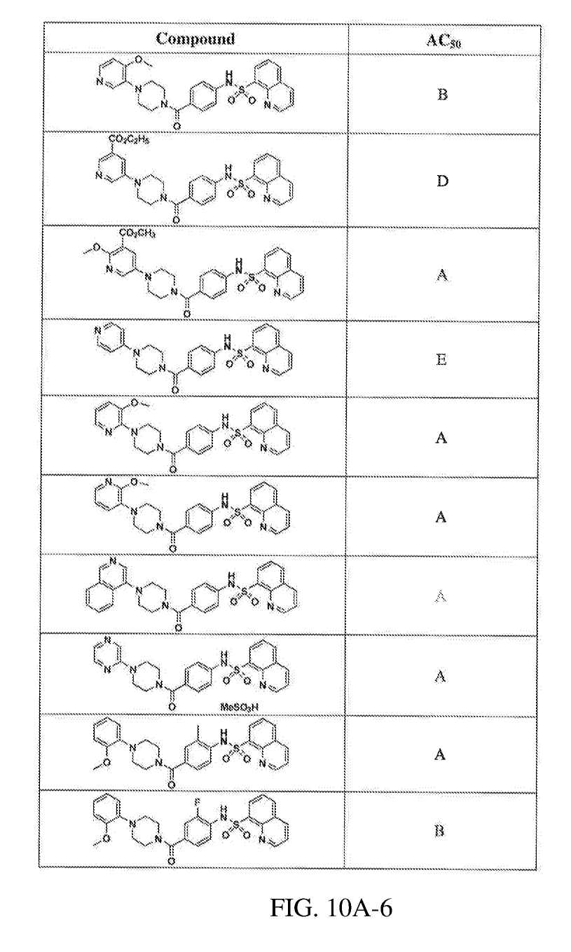

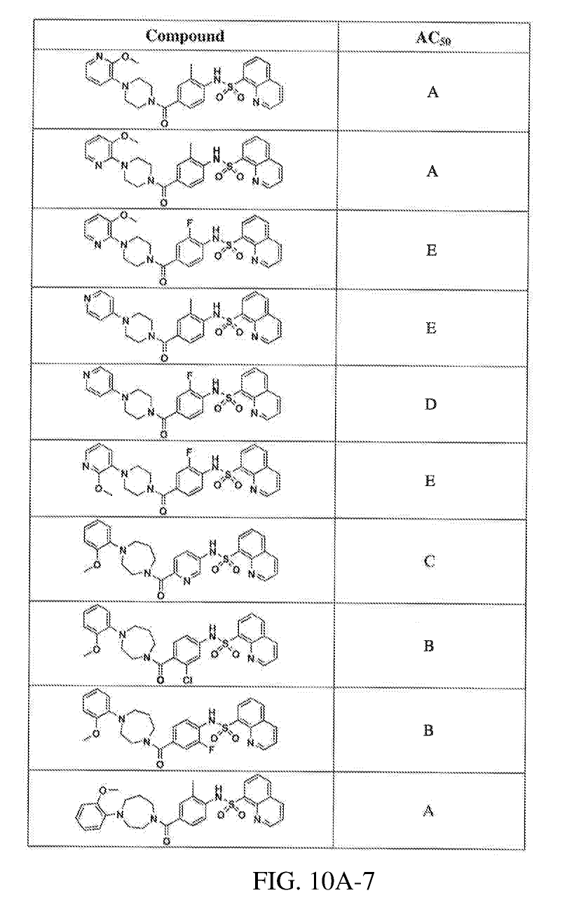

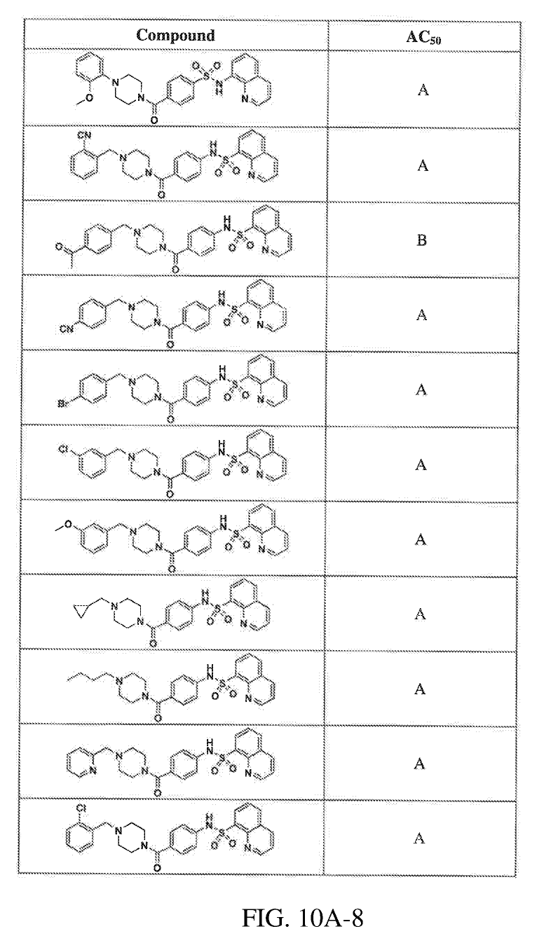

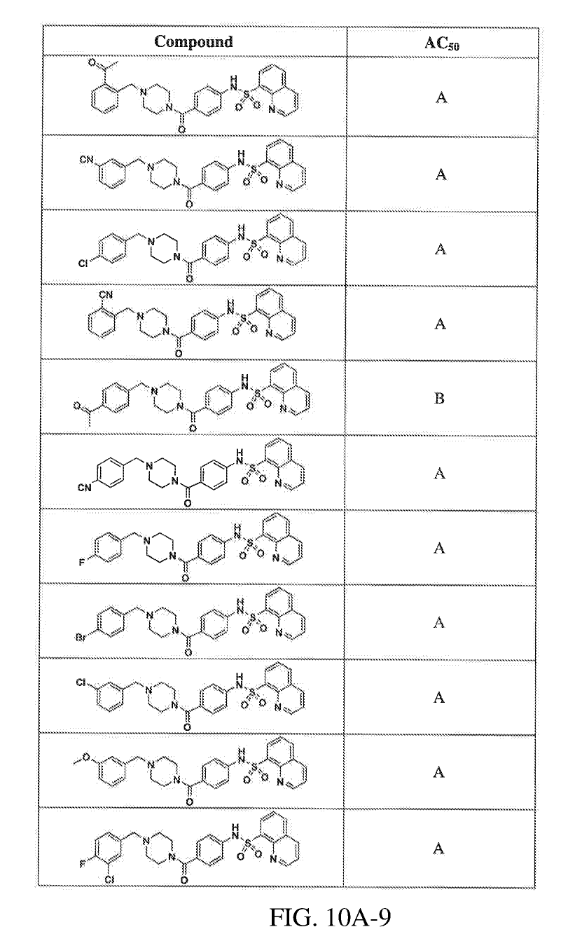

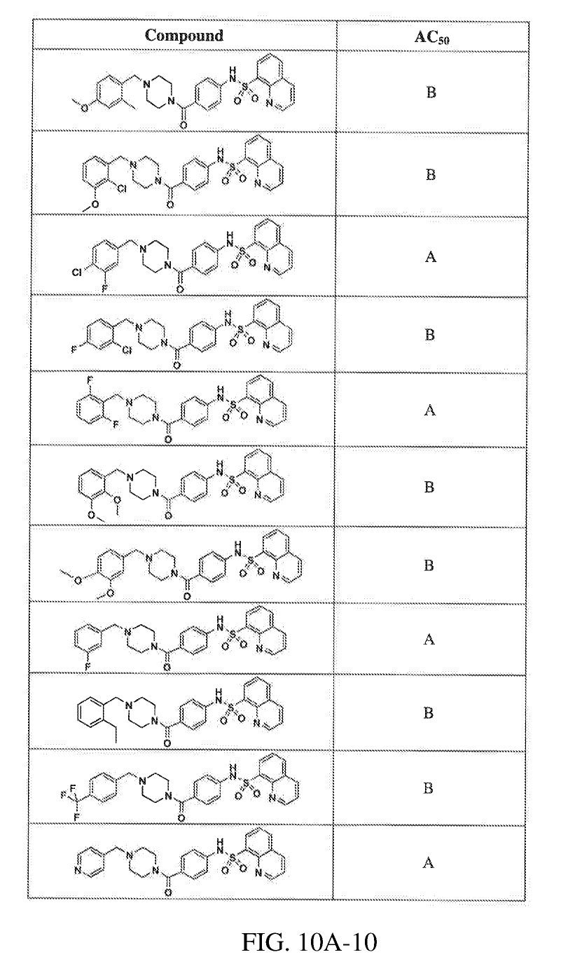

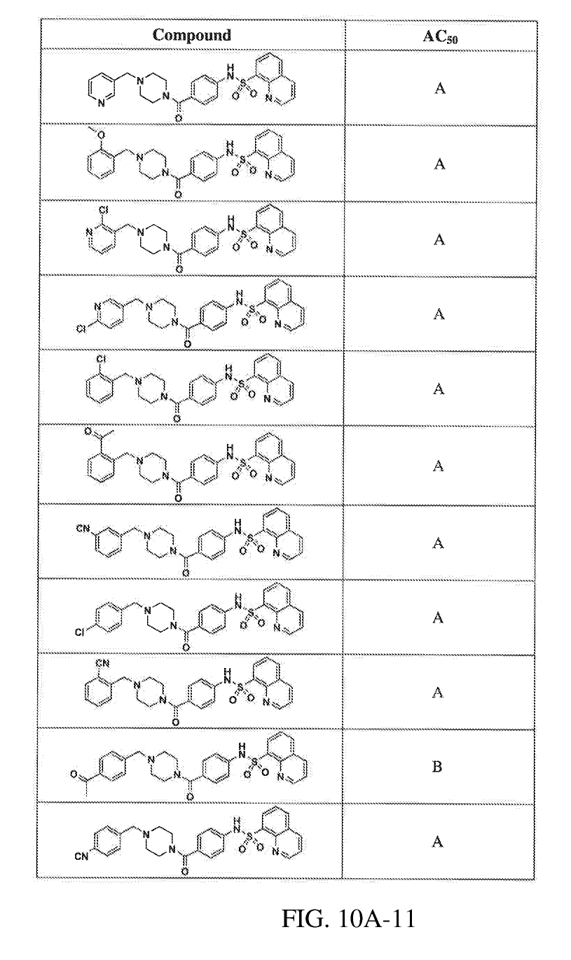

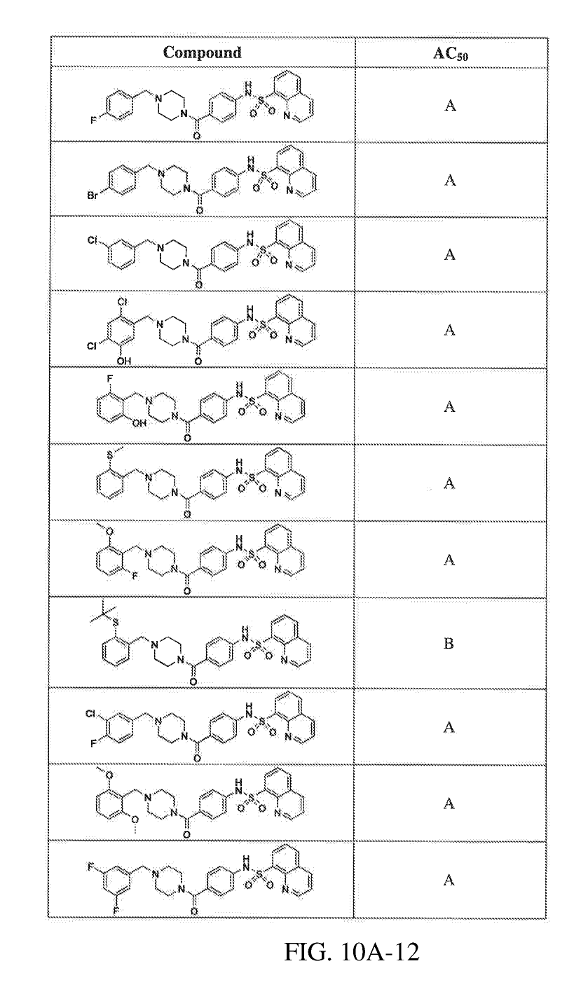

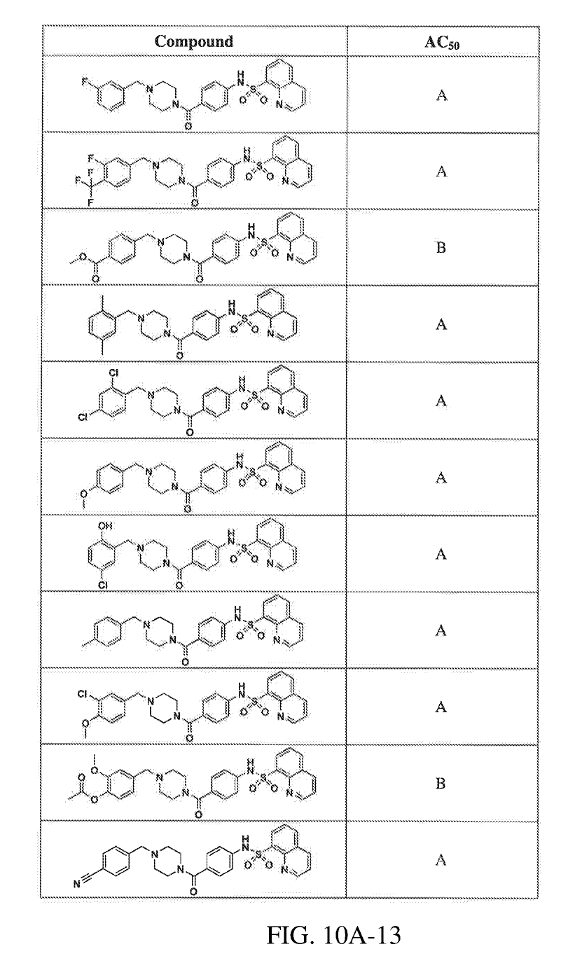

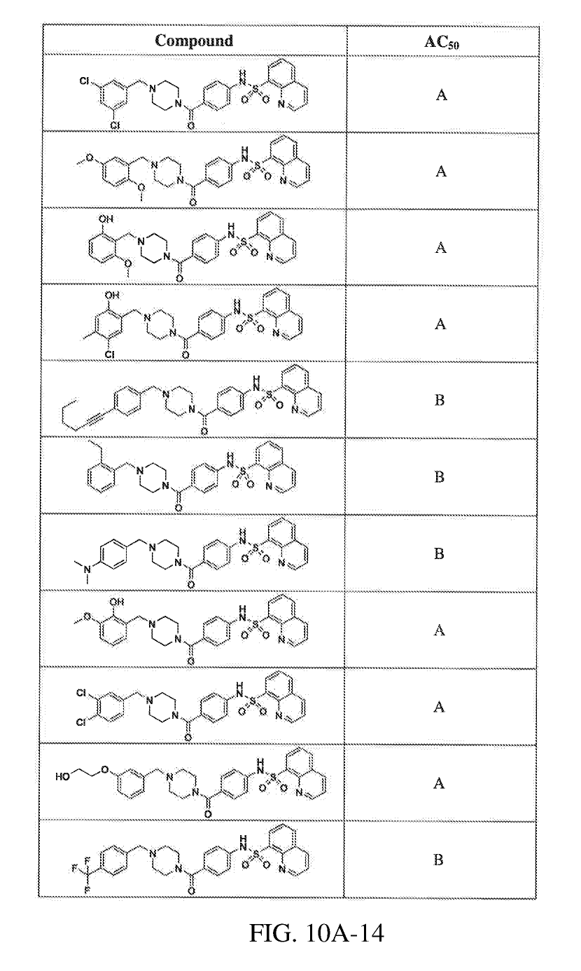

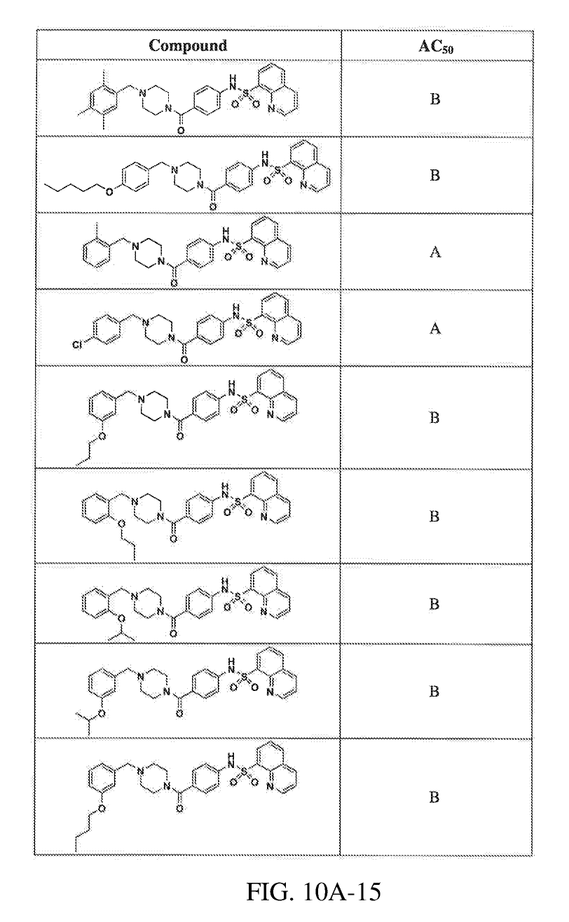

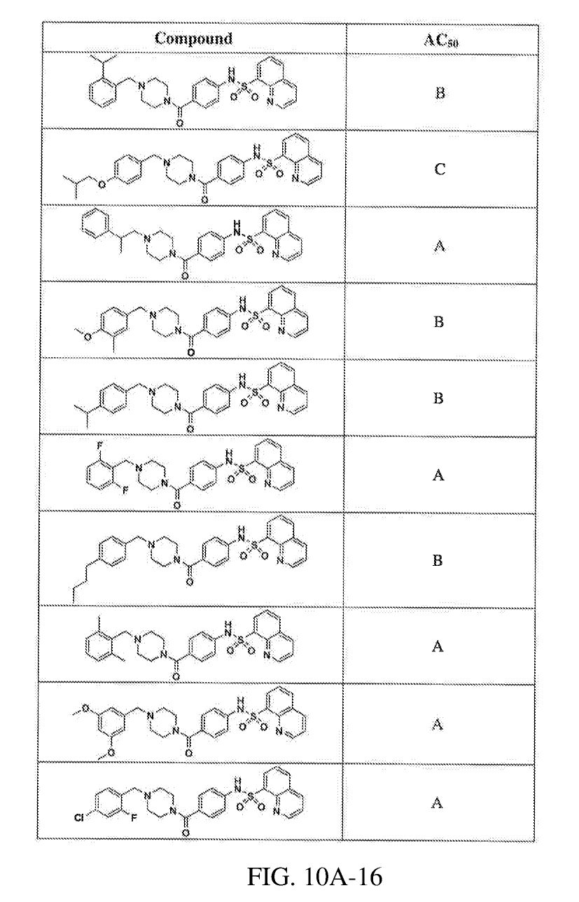

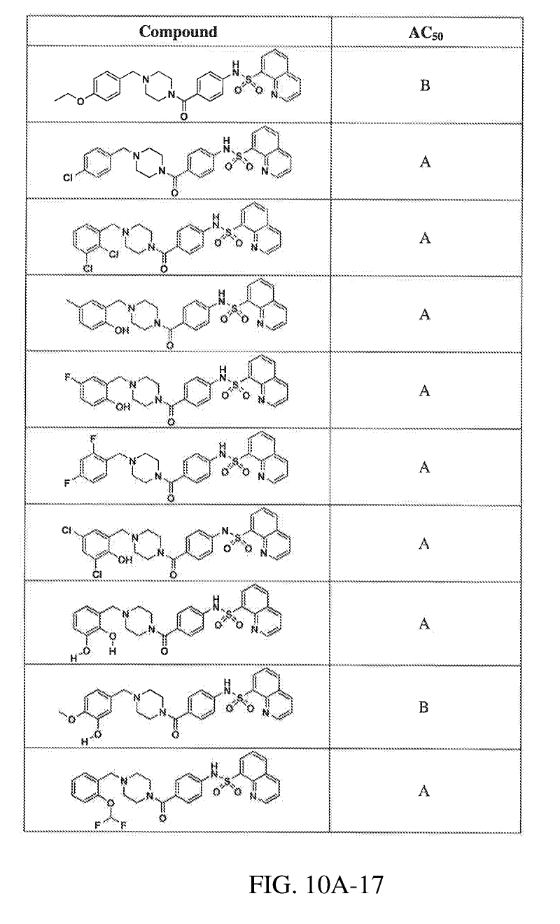

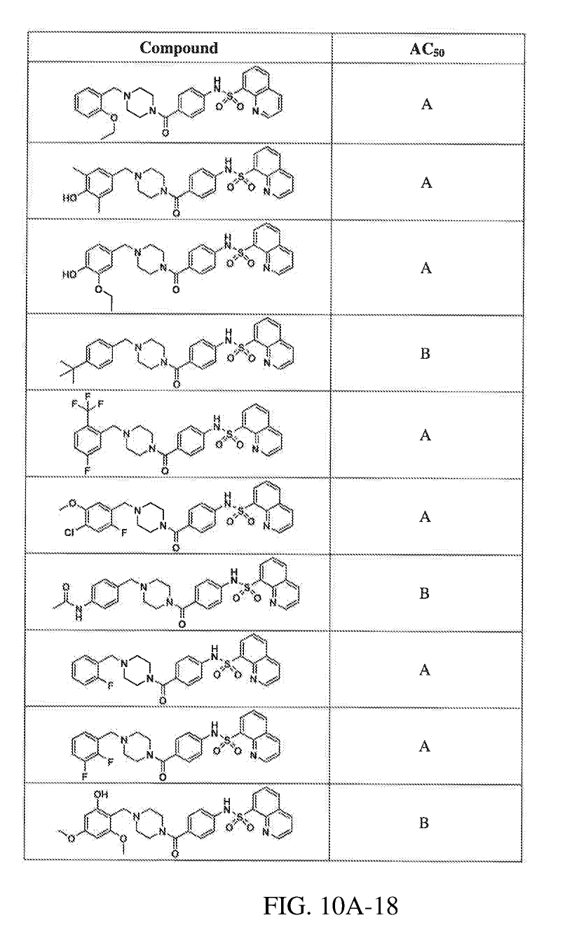

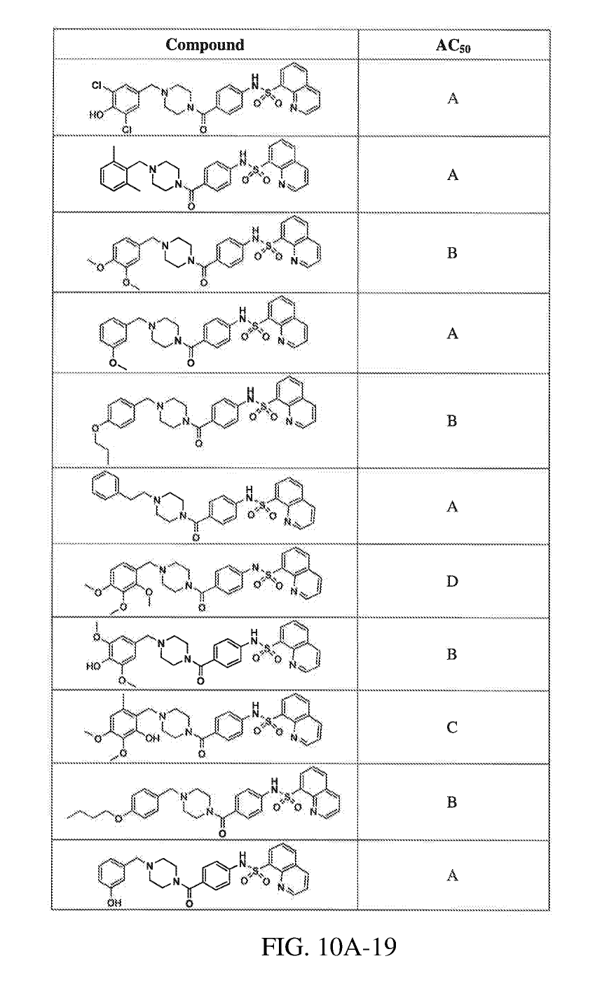

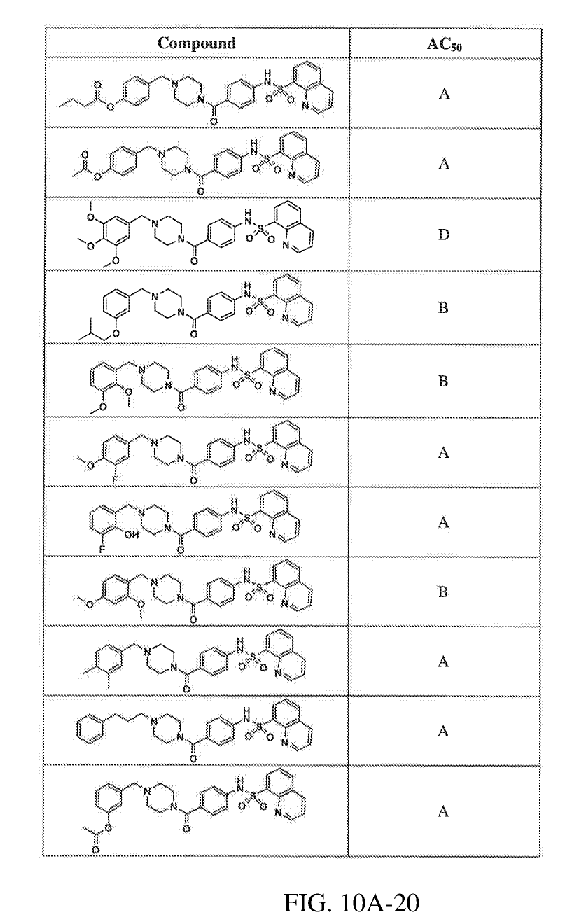

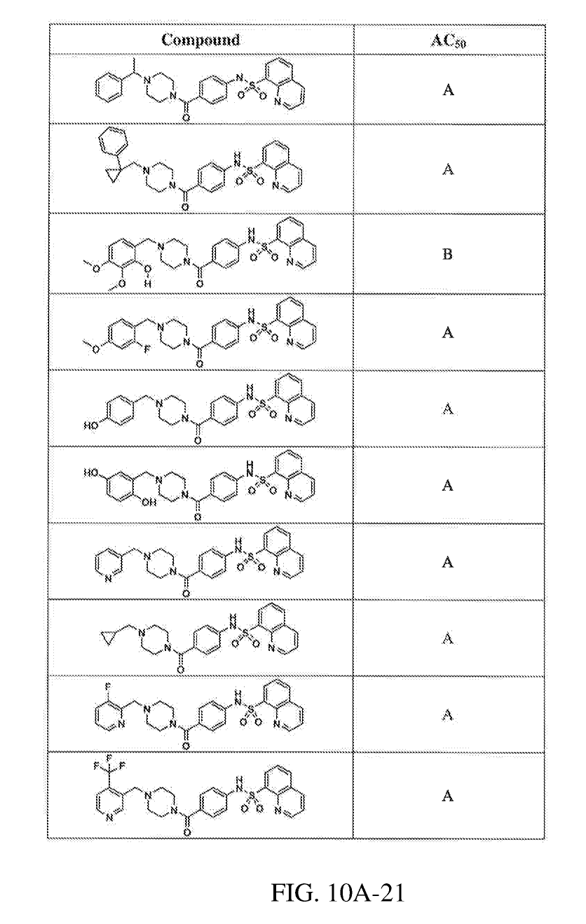

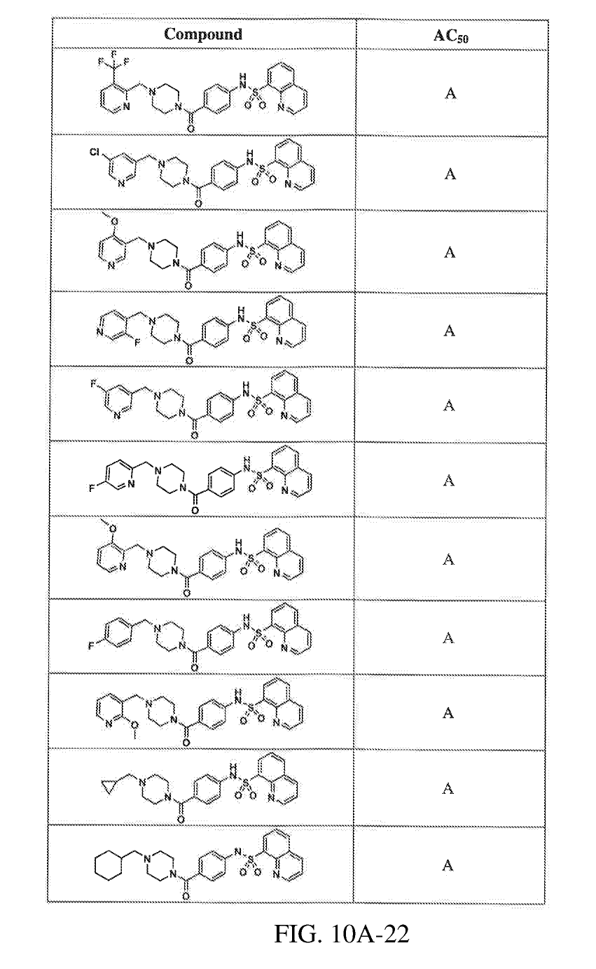

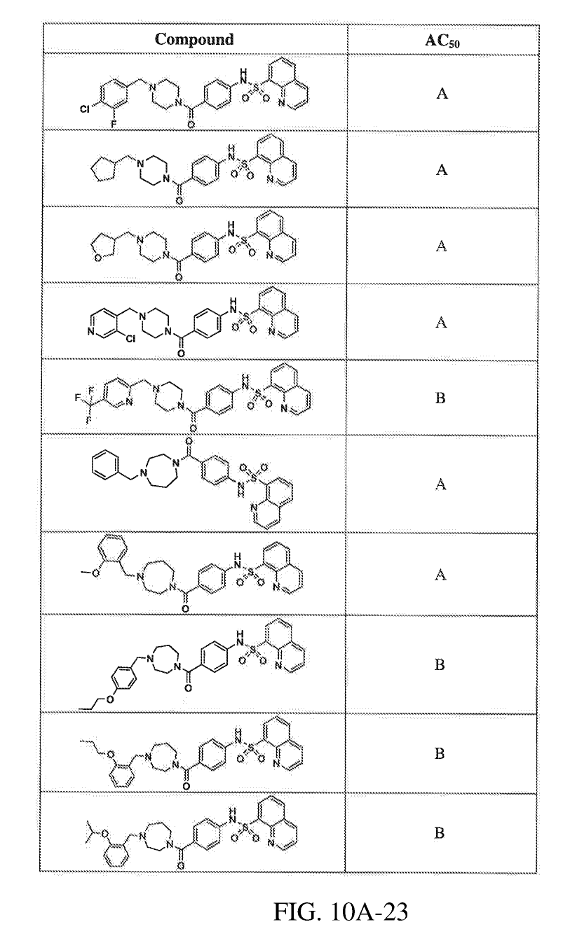

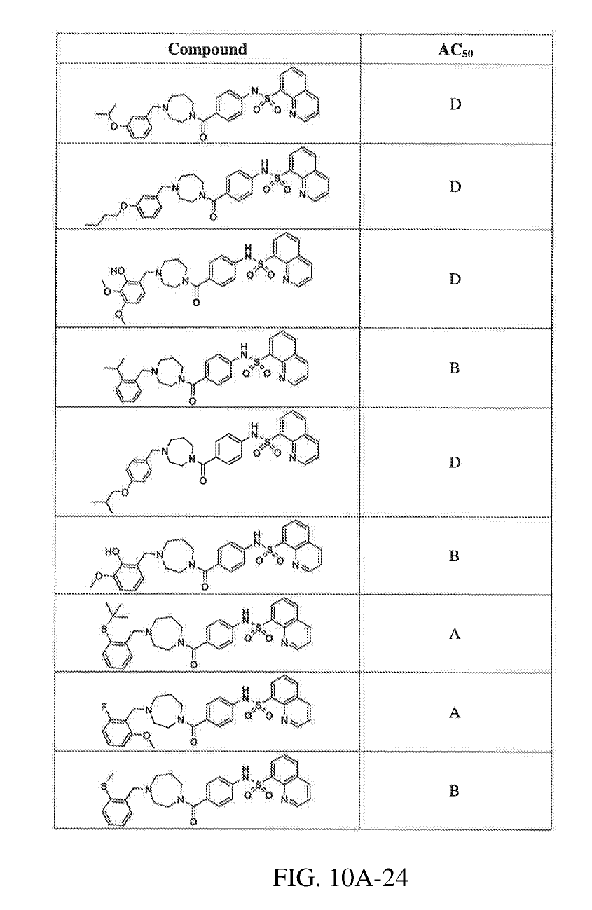

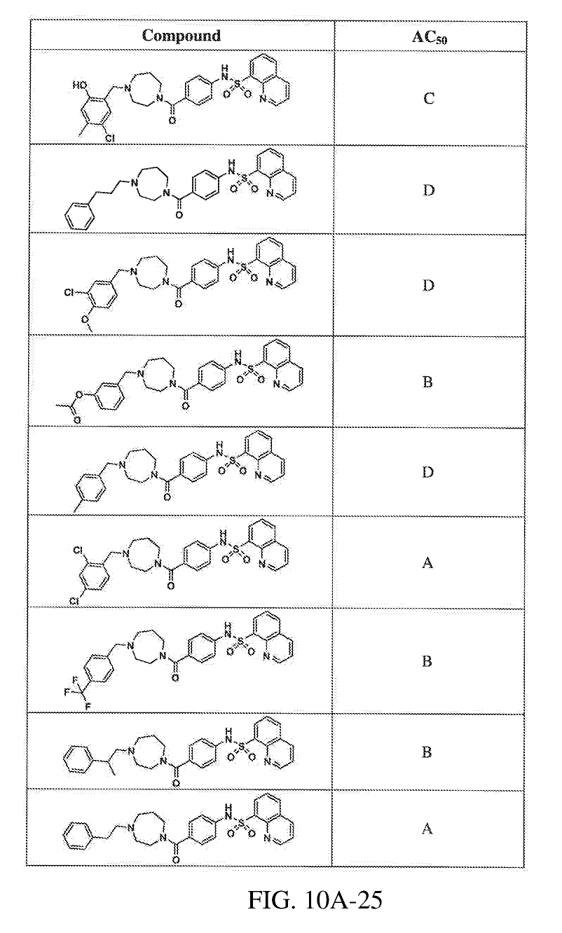

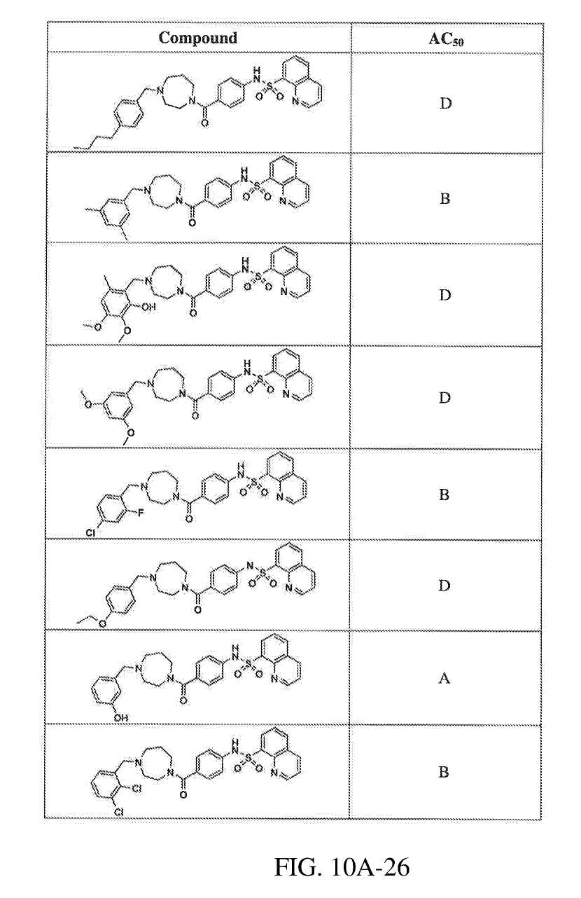

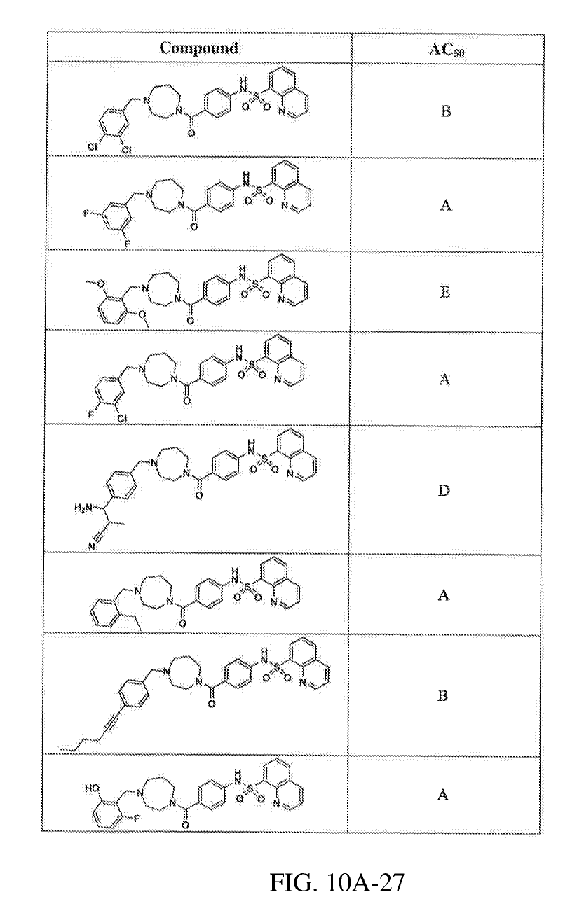

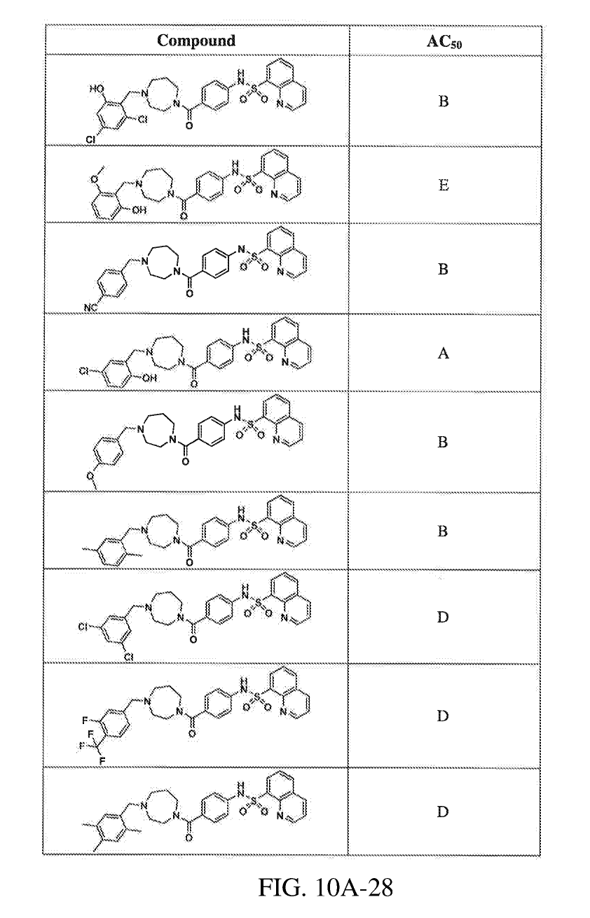

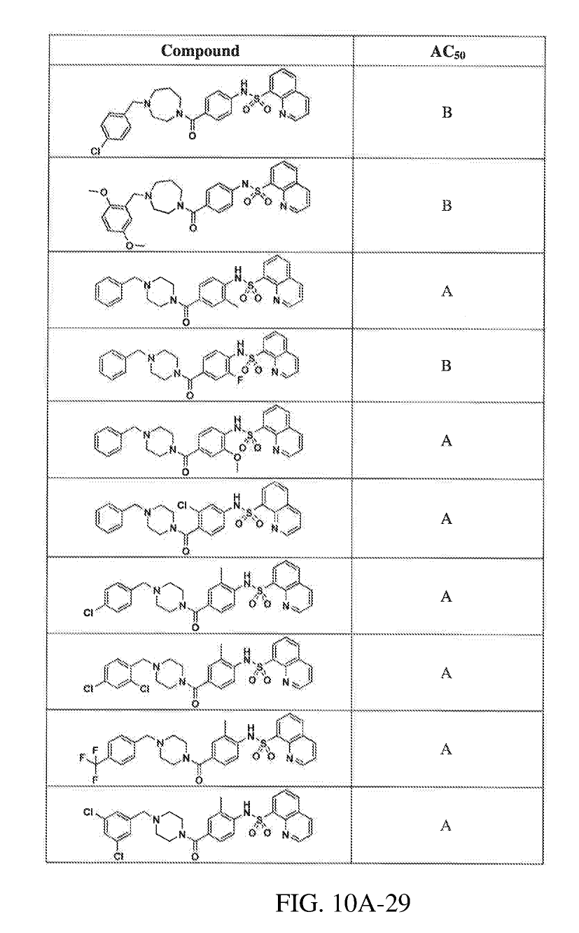

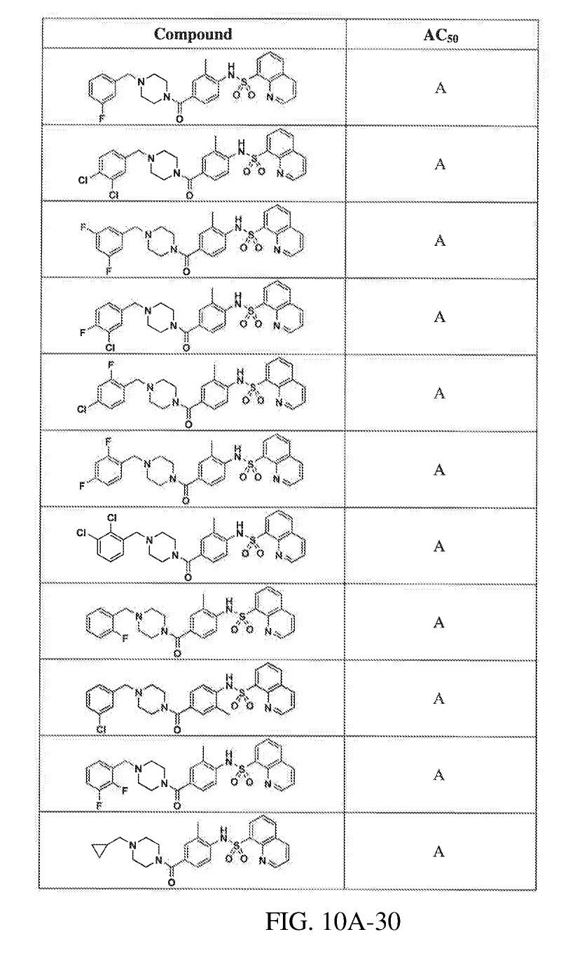

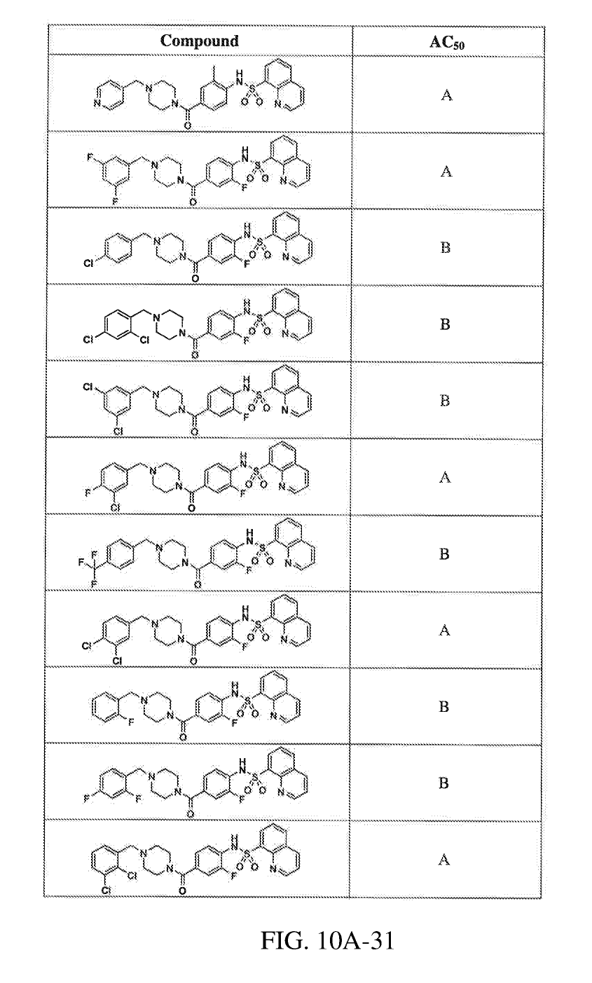

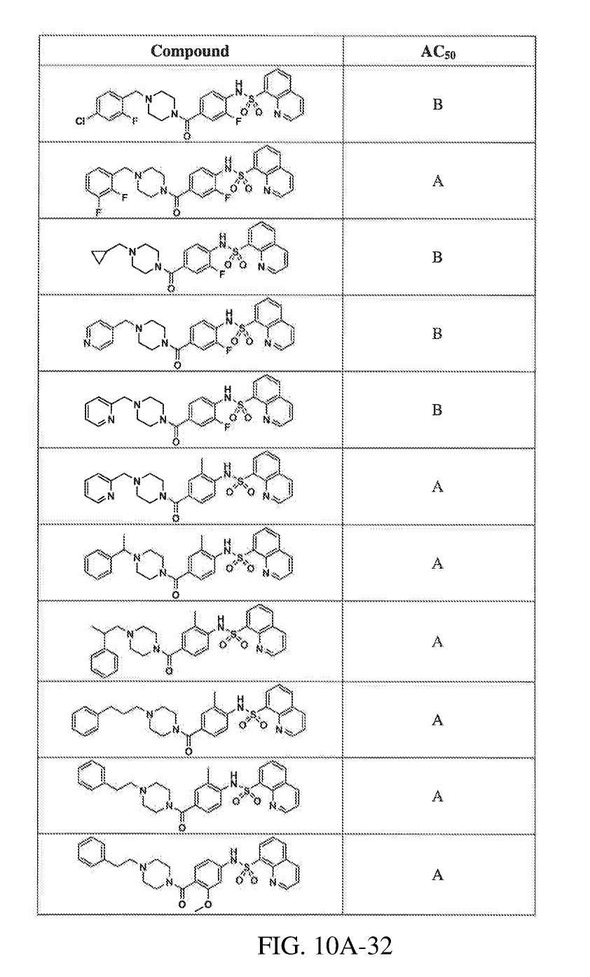

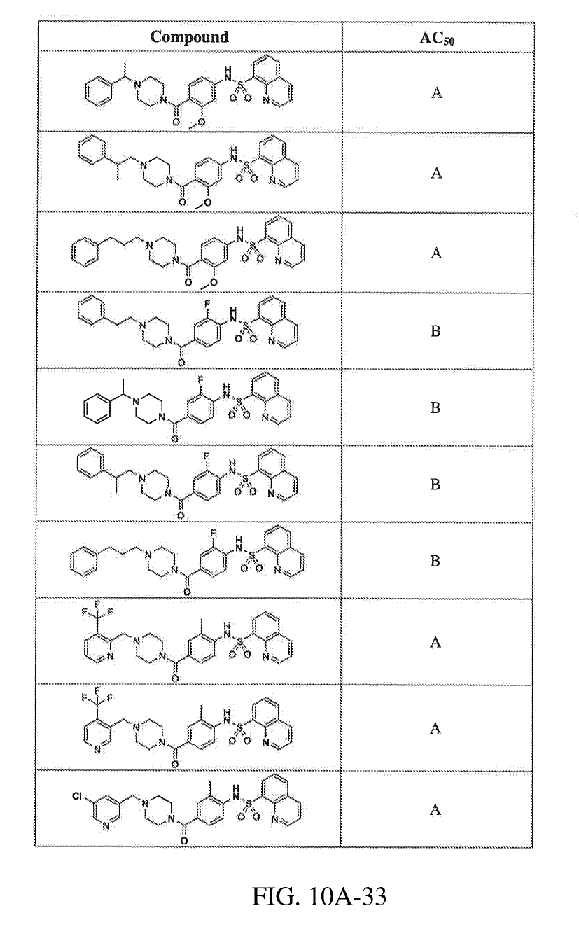

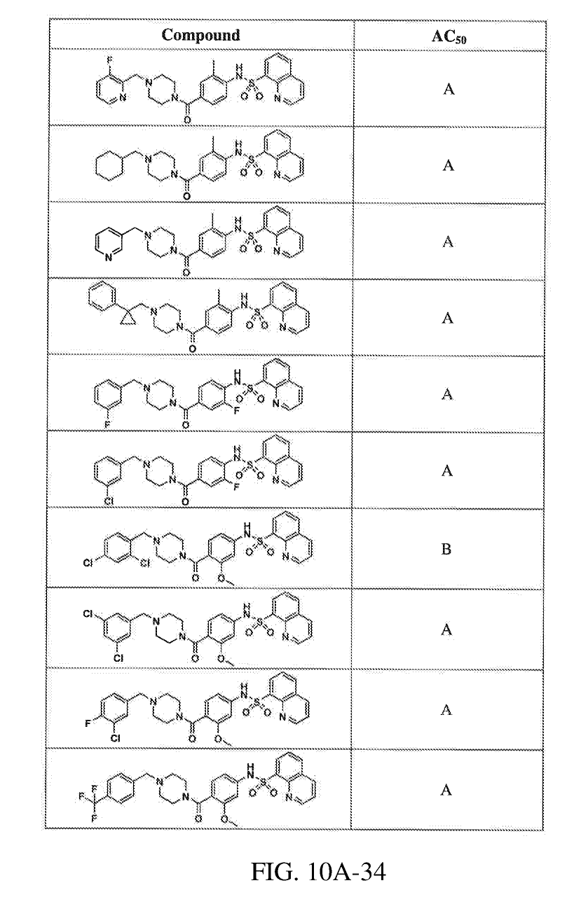

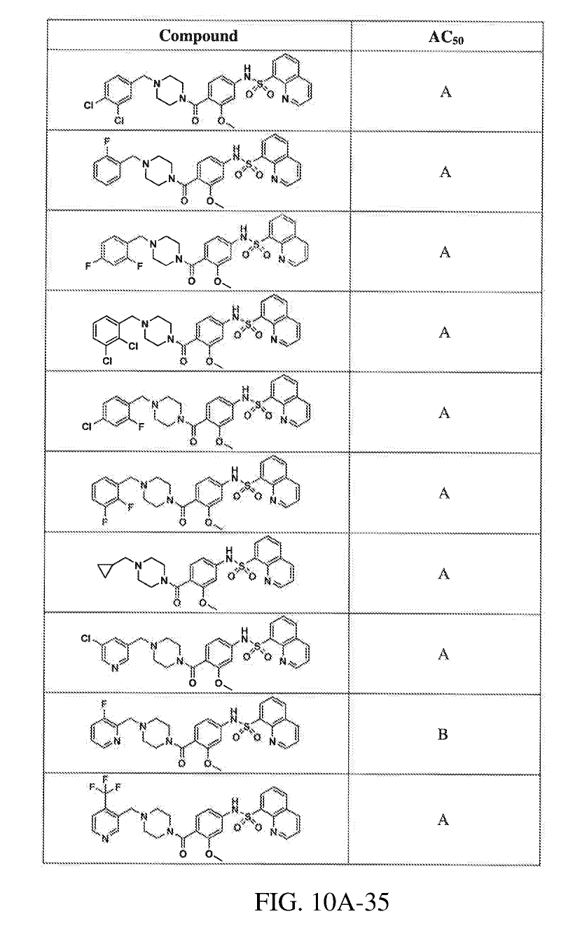

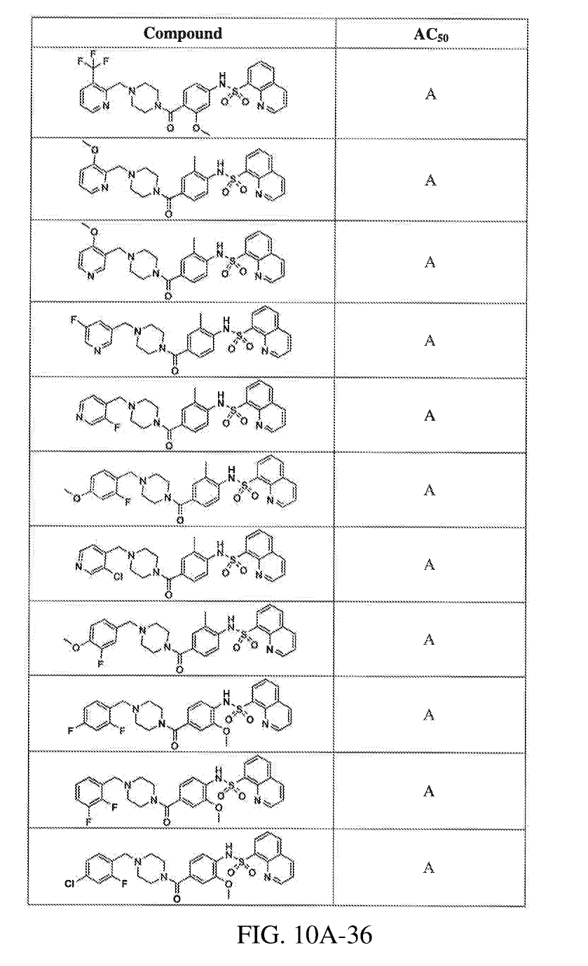

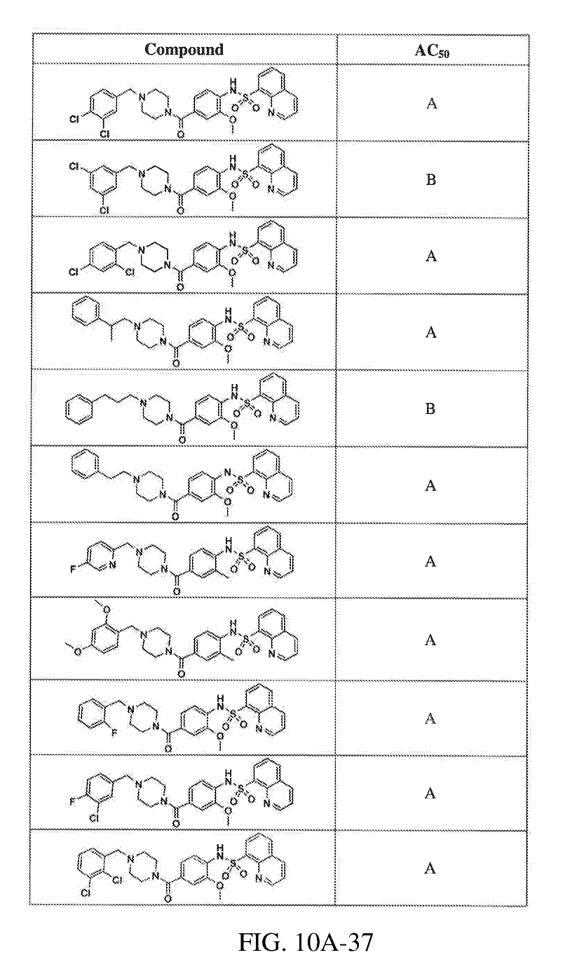

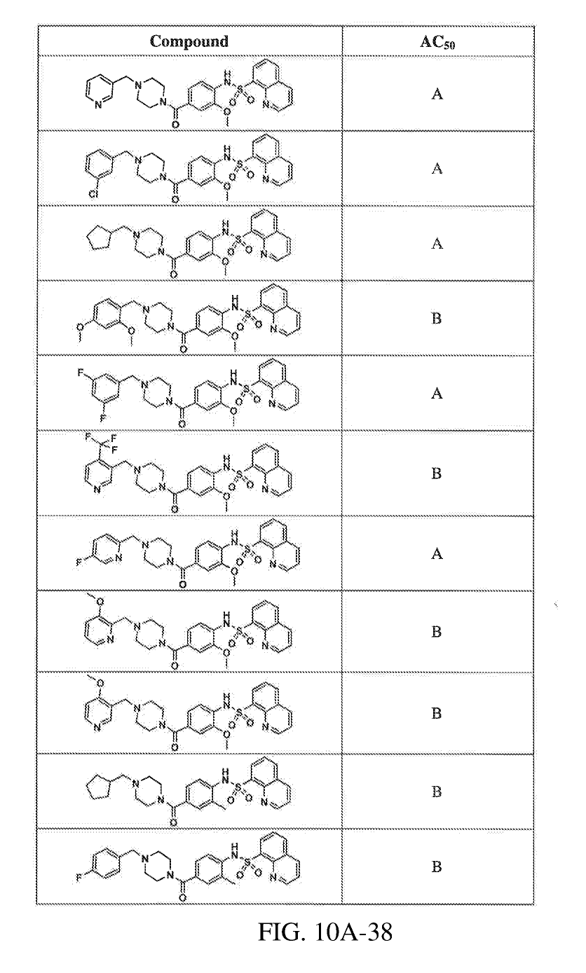

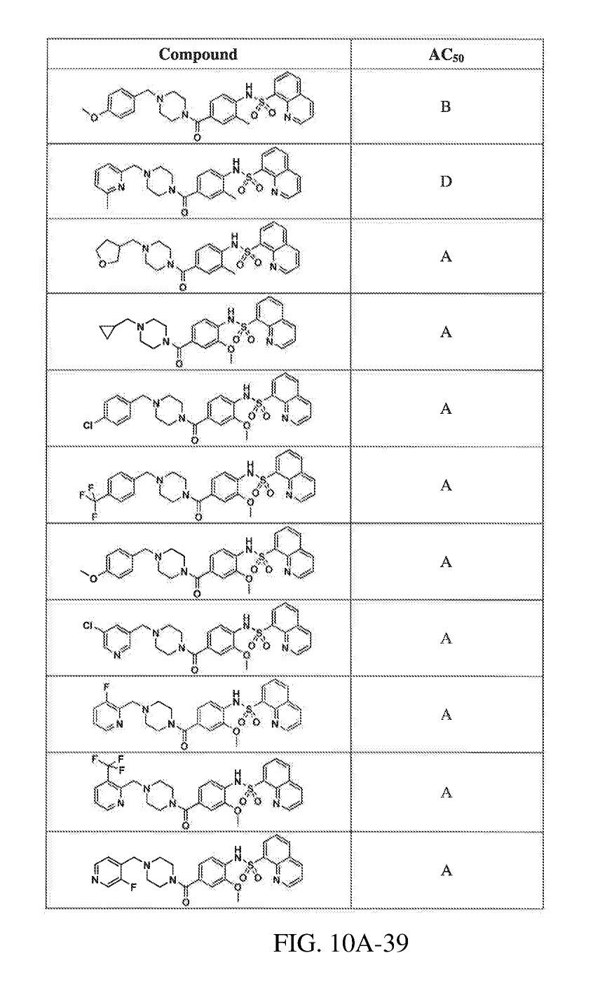

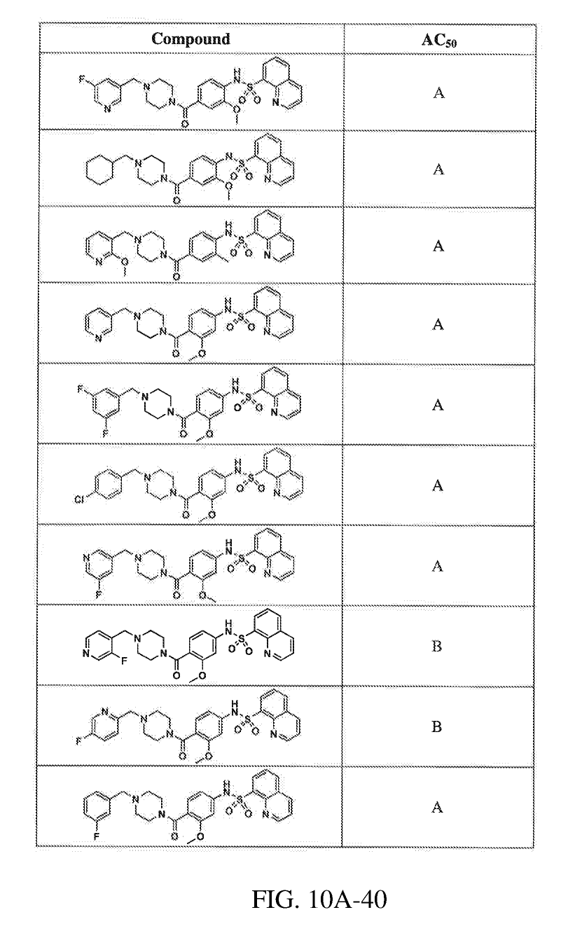

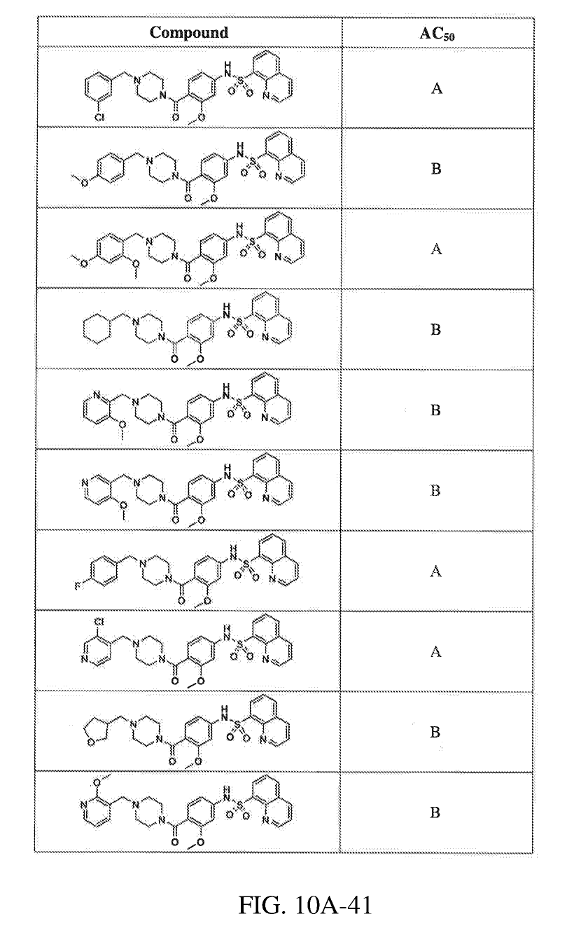

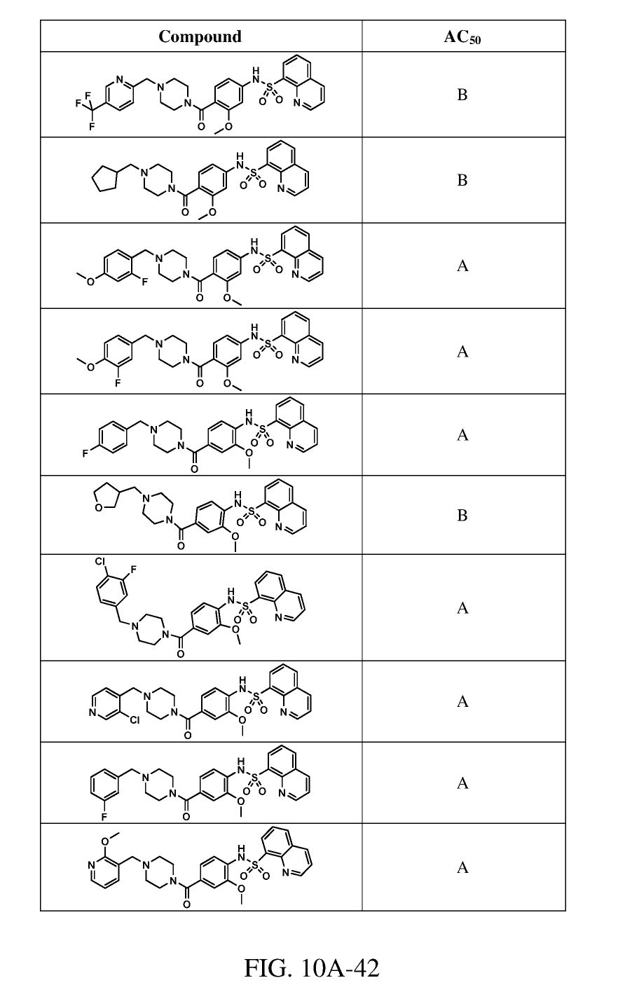

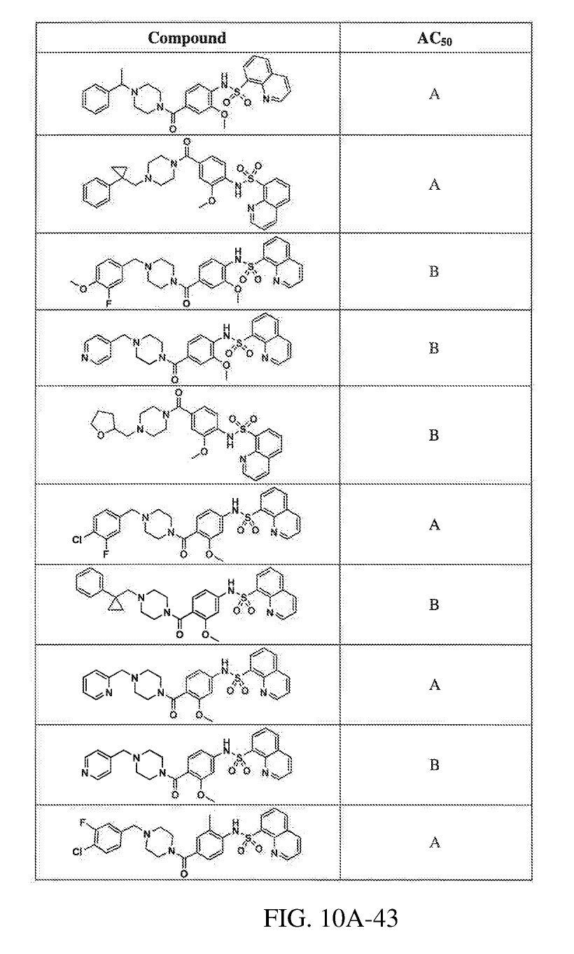

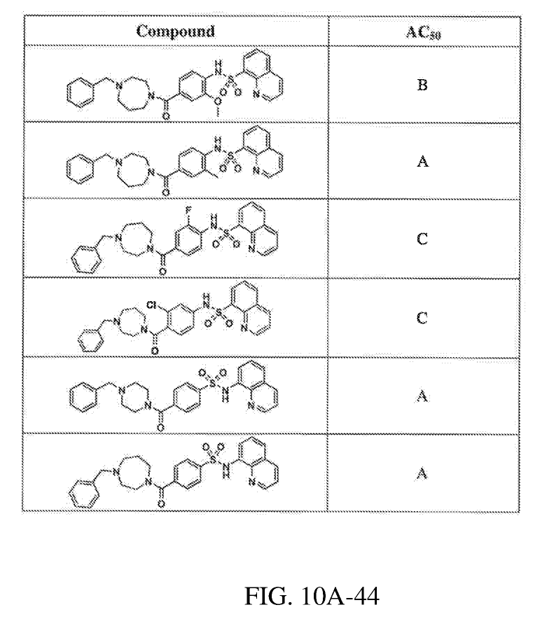

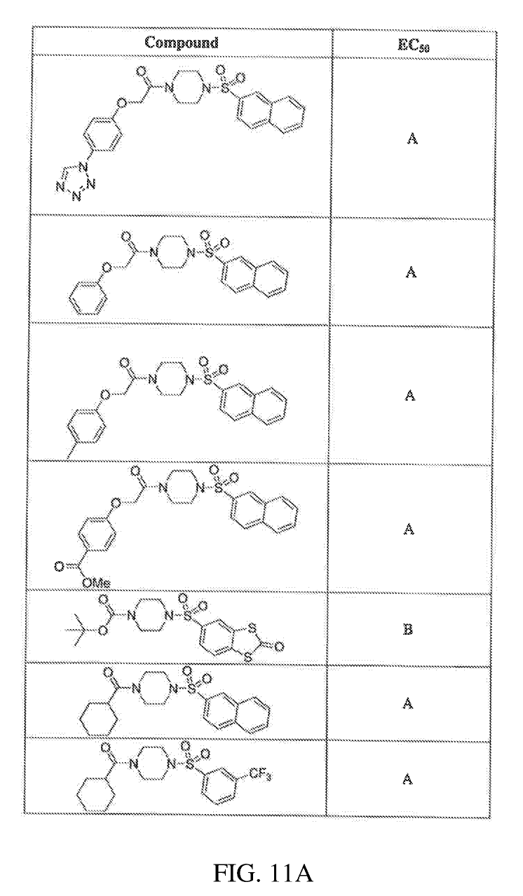

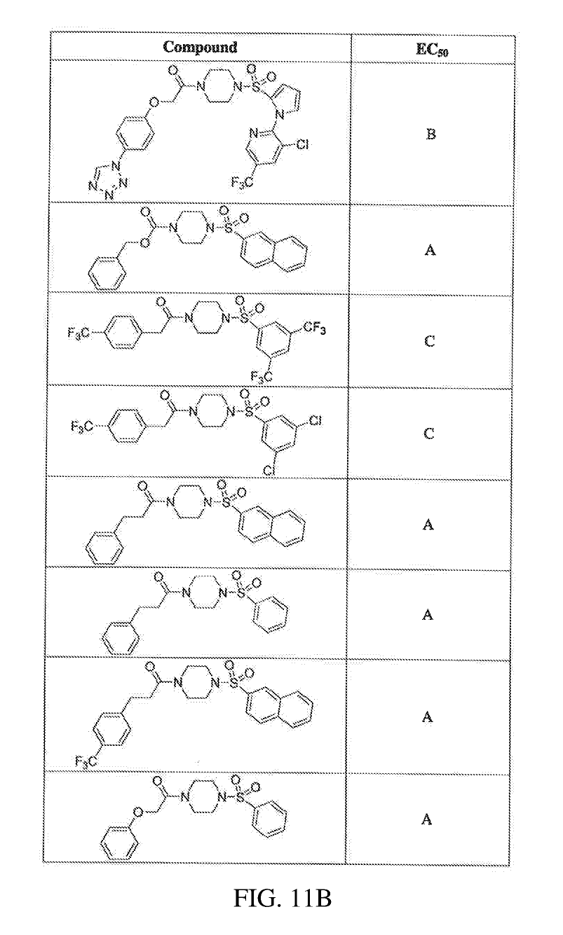

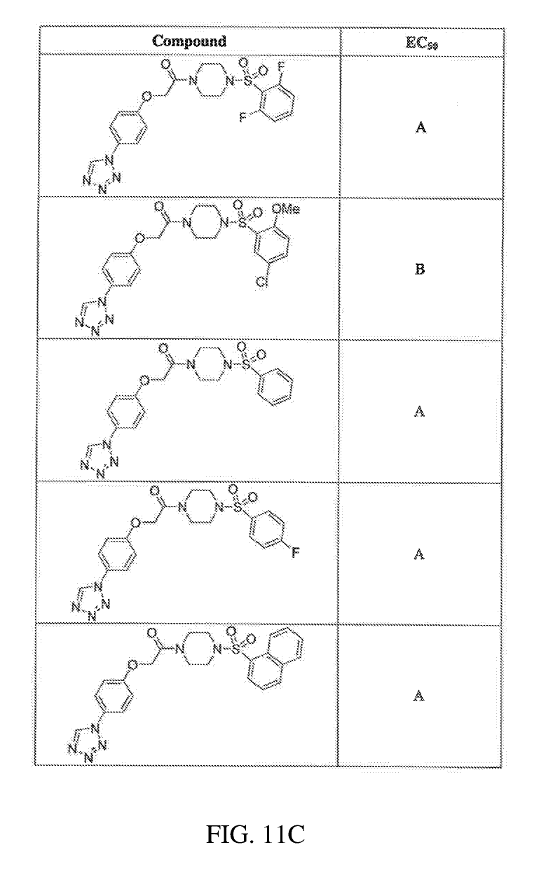

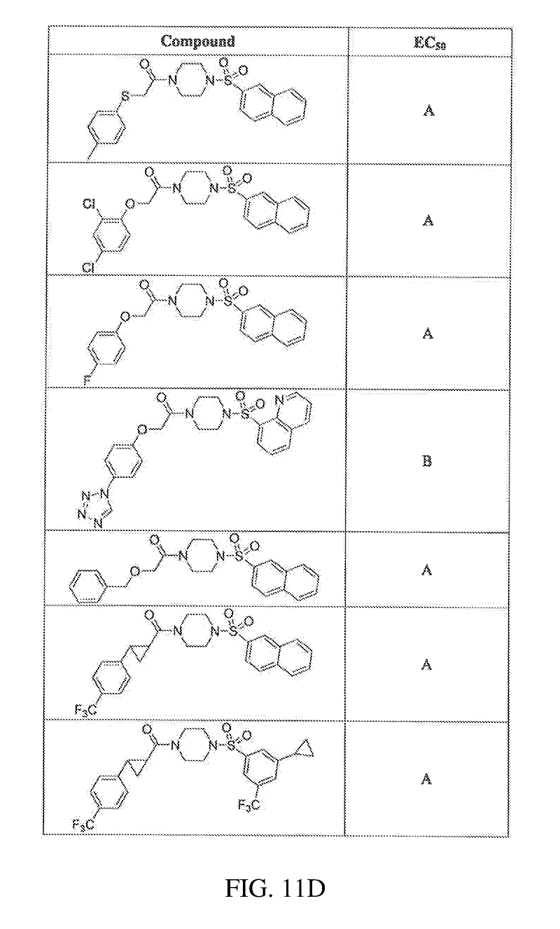

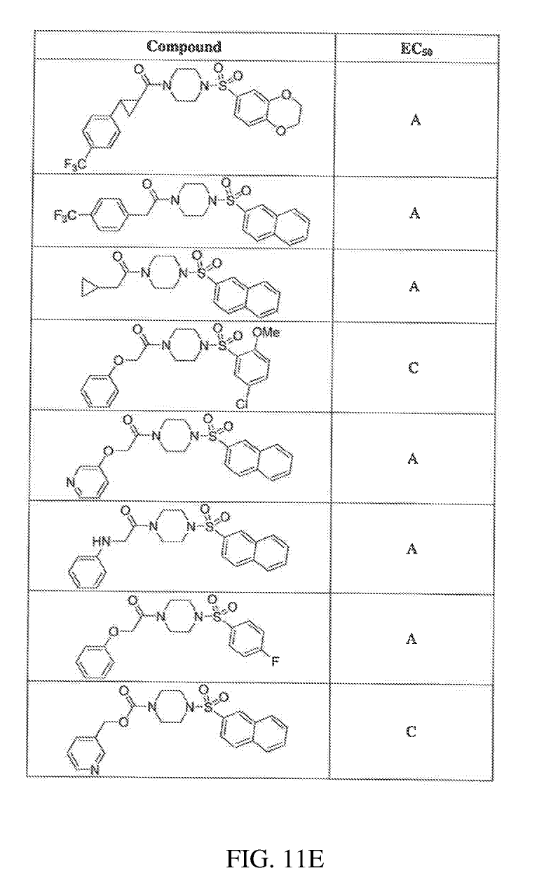

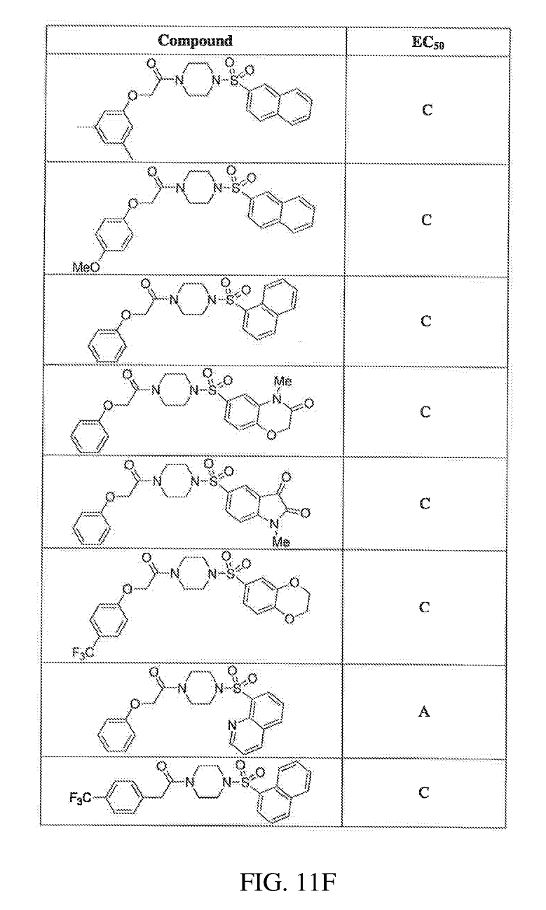

A compound described herein may be an activator of PKM2. Exemplary PKM2 activators include, but are not limited to the compounds of formulas (I), (II), (III), and (IV) as described herein. Exemplary compounds are shown in Table 1 which includes AGI-752, Table 2, FIGS. 10A-10A-44 or FIGS. 11A-11F. As shown in Table 1, A refers to an activator of PKM2 with an AC₅₀<100 nM. B refers to an activator of PKM2 with an AC₅₀ between 100 nM and 500 nM. C refers to an activator of PKM2 with an AC₅₀ greater than 500 nM. AC₅₀s described here in Tables 1 and 2 and FIGS. 1 and 2A-2D are measured according to the “PKM2 Assay Procedure” below.

### “PKM2 Assay Procedure”:

PKM2 stock enzyme solution was diluted in Reaction Buffer
2 μL of compound was added into each well first, and then 180 μL of the Reaction Mix was added.
Reaction mixture with compound (without ADP) were incubated for 30 minutes at 4° C.
Plates were re-equilibrated to room temperature prior to adding 20 μL ADP to initiate the reaction.
Reaction progress was measured as changes in absorbance at 340 nm wavelength at room temperature (25° C.)

### Reaction Mix:

PKM2 (50 ng/well), ADP (0.7 mM), PEP (0.15 mM), NADH (180 μM), LDH (2 units) in Reaction Buffer

Reaction Buffer:

100 mM KCl, 50 mM Tris pH 7.5, 5 mM MgCl2, 1 mM DTT, 0.03% BSA.

TABLE 1

| Compound | AC₅₀ |
|---|---|
| 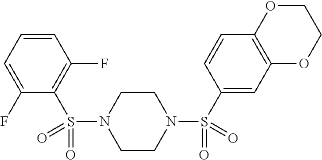 [SMILES: O=S(=O)(c1ccc2c(c1)OCCO2)N1CCN(S(=O)(=O)c2c(F)cccc2F)CC1] | A |
| 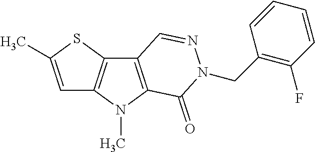 [SMILES: Cc1cc2c(s1)c1cnn(Cc3ccccc3F)c(=O)c1n2C] | A |
| 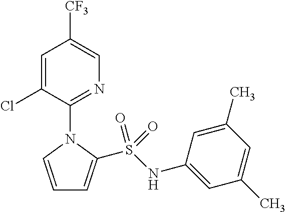 [SMILES: Cc1cc(C)cc(NS(=O)(=O)c2cccn2-c2ncc(C(F)(F)F)cc2Cl)c1] | A |
| 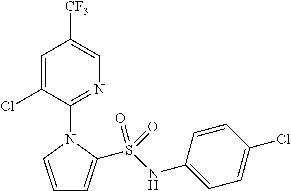 [SMILES: O=S(=O)(Nc1ccc(Cl)cc1)c1cccn1-c1ncc(C(F)(F)F)cc1Cl] | A |
| 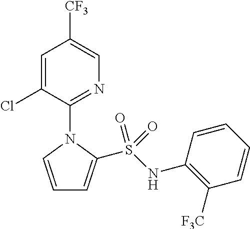 [SMILES: O=S(=O)(Nc1ccccc1C(F)(F)F)c1cccn1-c1ncc(C(F)(F)F)cc1Cl] | C |
| 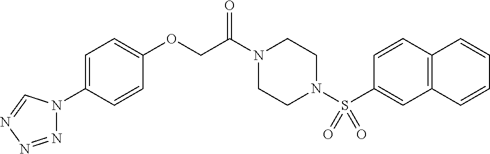 [SMILES: O=C(COc1ccc(-n2cnnn2)cc1)N1CCN(S(=O)(=O)c2ccc3ccccc3c2)CC1] | B |
| 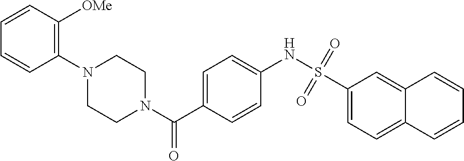 [SMILES: COc1ccccc1N1CCN(C(=O)c2ccc(NS(=O)(=O)c3ccc4ccccc4c3)cc2)CC1] | C |
| 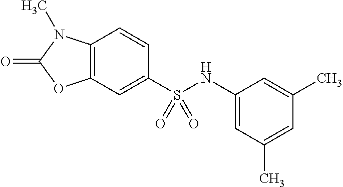 [SMILES: Cc1cc(C)cc(NS(=O)(=O)c2ccc3c(c2)oc(=O)n3C)c1] | A |
| 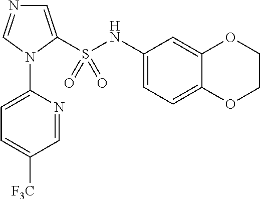 [SMILES: O=S(=O)(Nc1ccc2c(c1)OCCO2)c1cncn1-c1ccc(C(F)(F)F)cn1] | C |
| 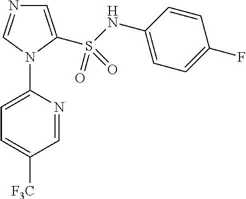 [SMILES: O=S(=O)(Nc1ccc(F)cc1)c1cncn1-c1ccc(C(F)(F)F)cn1] | C |
| 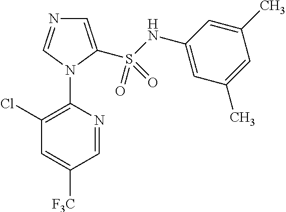 [SMILES: Cc1cc(C)cc(NS(=O)(=O)c2cncn2-c2ncc(C(F)(F)F)cc2Cl)c1] | A |
| 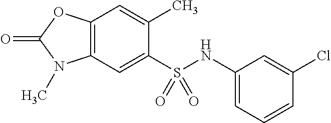 [SMILES: Cc1cc2oc(=O)n(C)c2cc1S(=O)(=O)Nc1cccc(Cl)c1] | B |
| 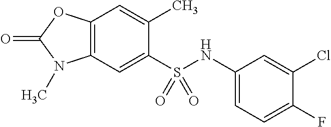 [SMILES: Cc1cc2oc(=O)n(C)c2cc1S(=O)(=O)Nc1ccc(F)c(Cl)c1] | A |
| 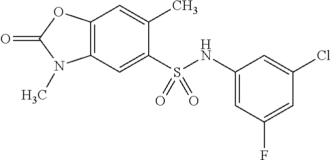 [SMILES: Cc1cc2oc(=O)n(C)c2cc1S(=O)(=O)Nc1cc(F)cc(Cl)c1] | C |
| 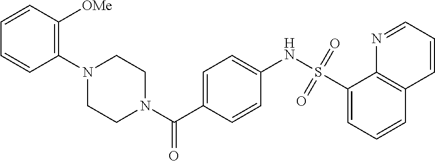 [SMILES: COc1ccccc1N1CCN(C(=O)c2ccc(NS(=O)(=O)c3cccc4cccnc34)cc2)CC1] | A |
| 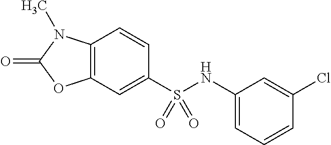 [SMILES: Cn1c(=O)oc2cc(S(=O)(=O)Nc3cccc(Cl)c3)ccc21] | A |
| 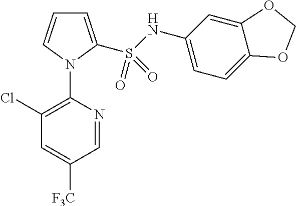 [SMILES: O=S(=O)(Nc1ccc2c(c1)OCO2)c1cccn1-c1ncc(C(F)(F)F)cc1Cl] | A |
| 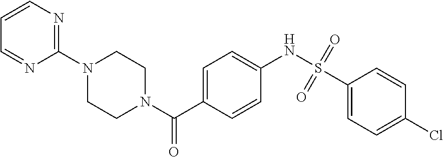 [SMILES: O=C(c1ccc(NS(=O)(=O)c2ccc(Cl)cc2)cc1)N1CCN(c2ncccn2)CC1] | C |
| 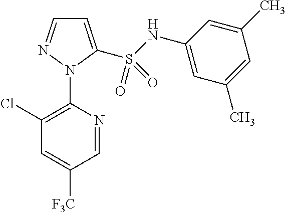 [SMILES: Cc1cc(C)cc(NS(=O)(=O)c2ccnn2-c2ncc(C(F)(F)F)cc2Cl)c1] | A |
| 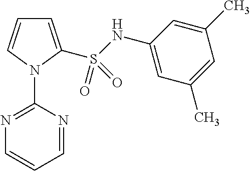 [SMILES: Cc1cc(C)cc(NS(=O)(=O)c2cccn2-c2ncccn2)c1] | A |
| 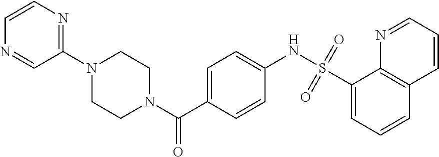 [SMILES: O=C(c1ccc(NS(=O)(=O)c2cccc3cccnc23)cc1)N1CCN(c2cnccn2)CC1] | A |
| 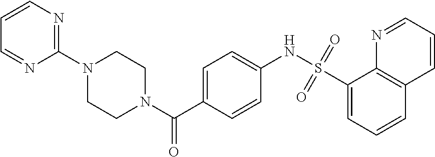 [SMILES: O=C(c1ccc(NS(=O)(=O)c2cccc3cccnc23)cc1)N1CCN(c2ncccn2)CC1] | B |
| 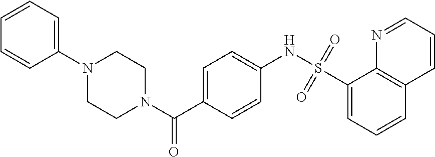 [SMILES: O=C(c1ccc(NS(=O)(=O)c2cccc3cccnc23)cc1)N1CCN(c2ccccc2)CC1] | A |
| 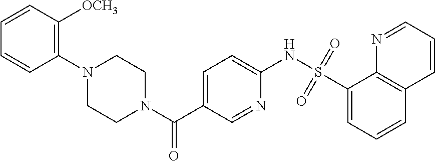 [SMILES: COc1ccccc1N1CCN(C(=O)c2ccc(NS(=O)(=O)c3cccc4cccnc34)nc2)CC1] | A |
| 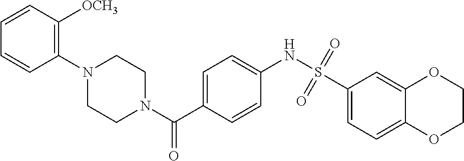 [SMILES: COc1ccccc1N1CCN(C(=O)c2ccc(NS(=O)(=O)c3ccc4c(c3)OCCO4)cc2)CC1] | B |
| 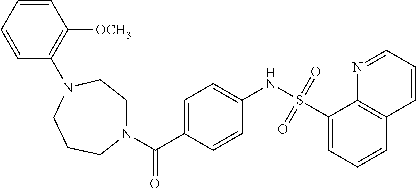 [SMILES: COc1ccccc1N1CCCN(C(=O)c2ccc(NS(=O)(=O)c3cccc4cccnc34)cc2)CC1] | C |
| 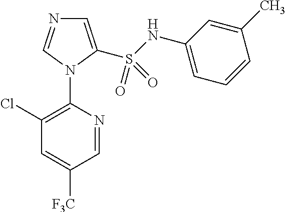 [SMILES: Cc1cccc(NS(=O)(=O)c2cncn2-c2ncc(C(F)(F)F)cc2Cl)c1] | A |
| 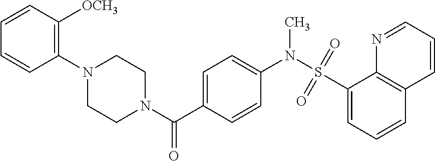 [SMILES: COc1ccccc1N1CCN(C(=O)c2ccc(N(C)S(=O)(=O)c3cccc4cccnc34)cc2)CC1] | A |
| 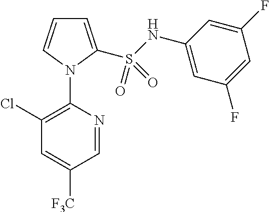 [SMILES: O=S(=O)(Nc1cc(F)cc(F)c1)c1cccn1-c1ncc(C(F)(F)F)cc1Cl] | C |
| 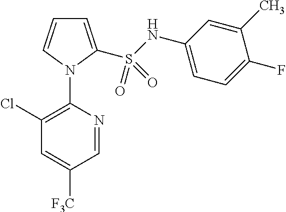 [SMILES: Cc1cc(NS(=O)(=O)c2cccn2-c2ncc(C(F)(F)F)cc2Cl)ccc1F] | A |
| 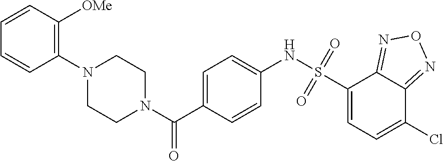 [SMILES: COc1ccccc1N1CCN(C(=O)c2ccc(NS(=O)(=O)c3ccc(Cl)c4nonc34)cc2)CC1] | C |
| 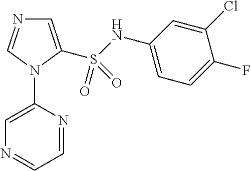 [SMILES: O=S(=O)(Nc1ccc(F)c(Cl)c1)c1cncn1-c1cnccn1] | A |
| 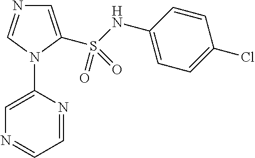 [SMILES: O=S(=O)(Nc1ccc(Cl)cc1)c1cncn1-c1cnccn1] | C |
| 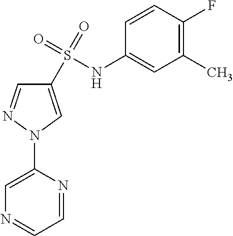 [SMILES: Cc1cc(NS(=O)(=O)c2cnn(-c3cnccn3)c2)ccc1F] | B |
| 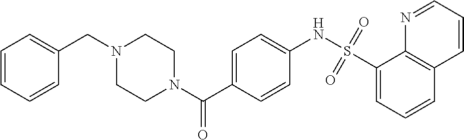 [SMILES: O=C(c1ccc(NS(=O)(=O)c2cccc3cccnc23)cc1)N1CCN(Cc2ccccc2)CC1] | A |
| 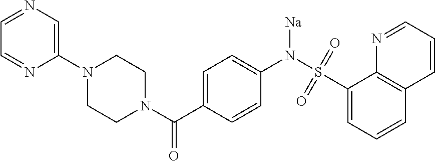 | A |
| 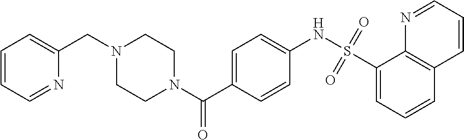 [SMILES: O=C(c1ccc(NS(=O)(=O)c2cccc3cccnc23)cc1)N1CCN(Cc2ccccn2)CC1] | A |
| 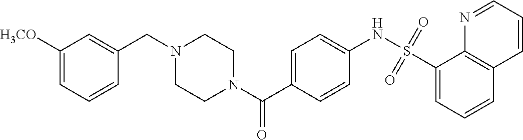 [SMILES: COc1cccc(CN2CCN(C(=O)c3ccc(NS(=O)(=O)c4cccc5cccnc45)cc3)CC2)c1] | A |
| 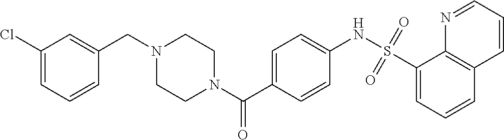 [SMILES: O=C(c1ccc(NS(=O)(=O)c2cccc3cccnc23)cc1)N1CCN(Cc2cccc(Cl)c2)CC1] | A |
| 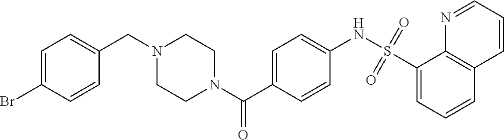 [SMILES: O=C(c1ccc(NS(=O)(=O)c2cccc3cccnc23)cc1)N1CCN(Cc2ccc(Br)cc2)CC1] | A |
| 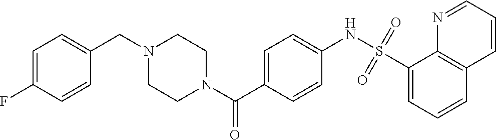 [SMILES: O=C(c1ccc(NS(=O)(=O)c2cccc3cccnc23)cc1)N1CCN(Cc2ccc(F)cc2)CC1] | A |
| 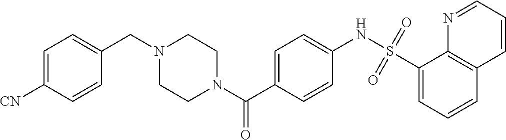 | A |
| 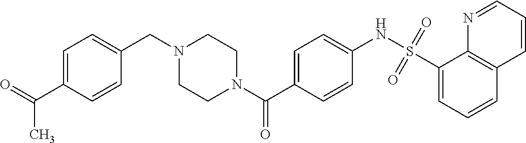 [SMILES: CC(=O)c1ccc(CN2CCN(C(=O)c3ccc(NS(=O)(=O)c4cccc5cccnc45)cc3)CC2)cc1] | B |
| 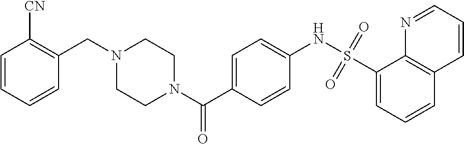 [SMILES: N#Cc1ccccc1CN1CCN(C(=O)c2ccc(NS(=O)(=O)c3cccc4cccnc34)cc2)CC1] | A |
| 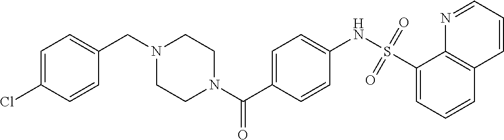 [SMILES: O=C(c1ccc(NS(=O)(=O)c2cccc3cccnc23)cc1)N1CCN(Cc2ccc(Cl)cc2)CC1] | A |
| 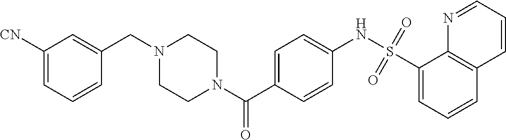 | A |
| 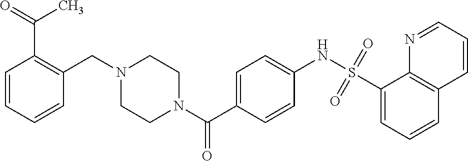 [SMILES: CC(=O)c1ccccc1CN1CCN(C(=O)c2ccc(NS(=O)(=O)c3cccc4cccnc34)cc2)CC1] | A |
| 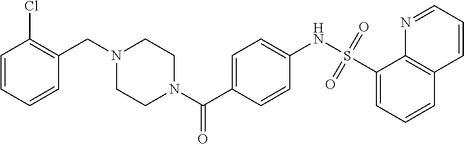 [SMILES: O=C(c1ccc(NS(=O)(=O)c2cccc3cccnc23)cc1)N1CCN(Cc2ccccc2Cl)CC1] | A |
| 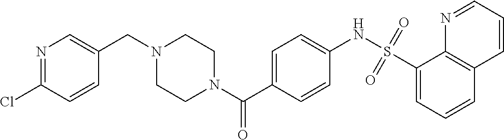 [SMILES: O=C(c1ccc(NS(=O)(=O)c2cccc3cccnc23)cc1)N1CCN(Cc2ccc(Cl)nc2)CC1] | A |
| 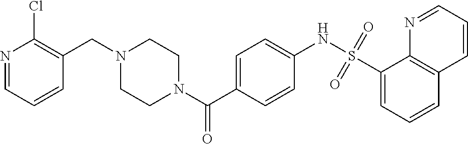 [SMILES: O=C(c1ccc(NS(=O)(=O)c2cccc3cccnc23)cc1)N1CCN(Cc2cccnc2Cl)CC1] | A |
| 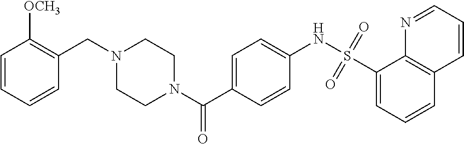 [SMILES: COc1ccccc1CN1CCN(C(=O)c2ccc(NS(=O)(=O)c3cccc4cccnc34)cc2)CC1] | A |
| 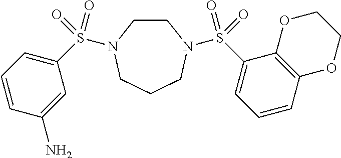 [SMILES: Nc1cccc(S(=O)(=O)N2CCCN(S(=O)(=O)c3cccc4c3OCCO4)CC2)c1] | A |

Exemplary compounds are also shown in Table 2. As shown in Table 2, A refers to an activator of PKM2 with an AC₅₀<1 μM. B refers to an activator of PKM2 with an AC₅₀ between 1 μM and 10 μM. C refers to an activator of PKM2 with an AC₅₀ between 10 μM and 50 μM. C refers to an activator of PKM2 with an AC₅₀ between 50 μM and 100 μM. D refers to an activator of PKM2 with an AC₅₀>100 μM. E refers to an activator of PKM2 that has not been tested.

TABLE 2

| Structure | AC₅₀ |
|---|---|
| 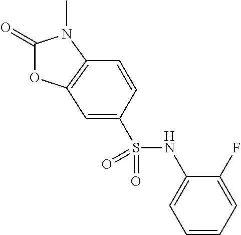 [SMILES: Cn1c(=O)oc2cc(S(=O)(=O)Nc3ccccc3F)ccc21] | E |
| 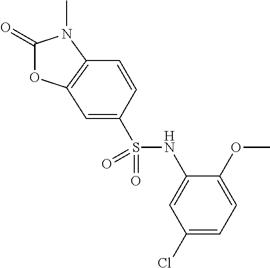 [SMILES: COc1ccc(Cl)cc1NS(=O)(=O)c1ccc2c(c1)oc(=O)n2C] | B |
| 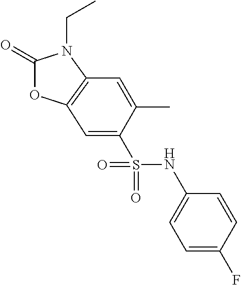 [SMILES: CCn1c(=O)oc2cc(S(=O)(=O)Nc3ccc(F)cc3)c(C)cc21] | C |
| 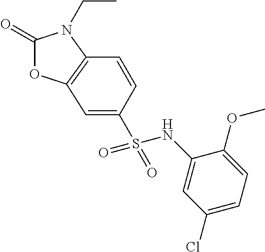 [SMILES: CCn1c(=O)oc2cc(S(=O)(=O)Nc3cc(Cl)ccc3OC)ccc21] | E |
| 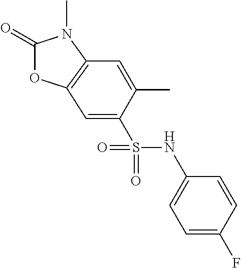 [SMILES: Cc1cc2c(cc1S(=O)(=O)Nc1ccc(F)cc1)oc(=O)n2C] | B |
| 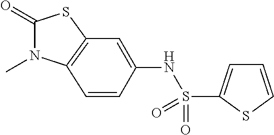 [SMILES: Cn1c(=O)sc2cc(NS(=O)(=O)c3cccs3)ccc21] | C |
| 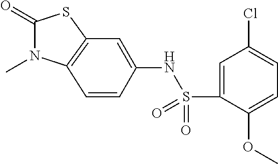 [SMILES: COc1ccc(Cl)cc1S(=O)(=O)Nc1ccc2c(c1)sc(=O)n2C] | C |
| 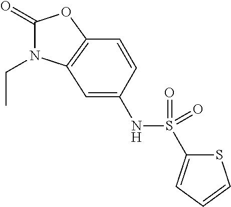 [SMILES: CCn1c(=O)oc2ccc(NS(=O)(=O)c3cccs3)cc21] | D |
| 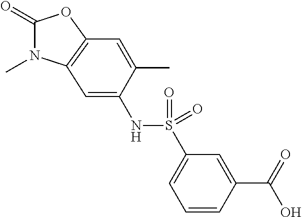 [SMILES: Cc1cc2oc(=O)n(C)c2cc1NS(=O)(=O)c1cccc(C(=O)O)c1] | E |
| 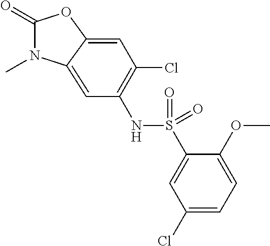 [SMILES: COc1ccc(Cl)cc1S(=O)(=O)Nc1cc2c(cc1Cl)oc(=O)n2C] | B |
| 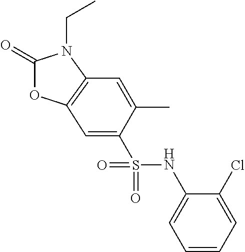 [SMILES: CCn1c(=O)oc2cc(S(=O)(=O)Nc3ccccc3Cl)c(C)cc21] | B |
| 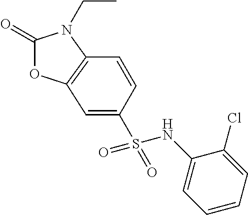 [SMILES: CCn1c(=O)oc2cc(S(=O)(=O)Nc3ccccc3Cl)ccc21] | B |
| 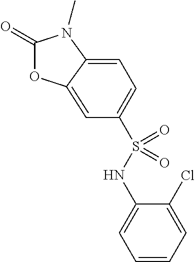 [SMILES: Cn1c(=O)oc2cc(S(=O)(=O)Nc3ccccc3Cl)ccc21] | B |
| 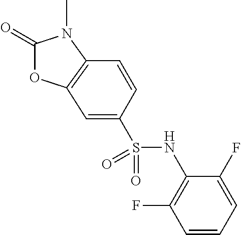 [SMILES: Cn1c(=O)oc2cc(S(=O)(=O)Nc3c(F)cccc3F)ccc21] | B |
| 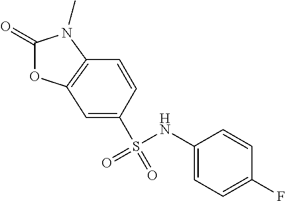 [SMILES: Cn1c(=O)oc2cc(S(=O)(=O)Nc3ccc(F)cc3)ccc21] | B |
| 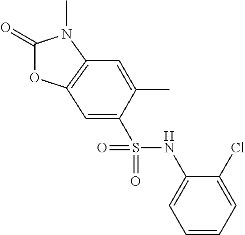 [SMILES: Cc1cc2c(cc1S(=O)(=O)Nc1ccccc1Cl)oc(=O)n2C] | B |
| 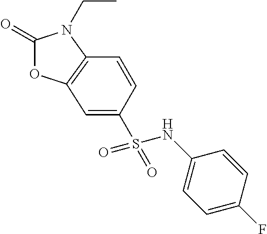 [SMILES: CCn1c(=O)oc2cc(S(=O)(=O)Nc3ccc(F)cc3)ccc21] | B |
| 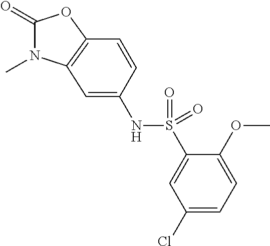 [SMILES: COc1ccc(Cl)cc1S(=O)(=O)Nc1ccc2oc(=O)n(C)c2c1] | D |
| 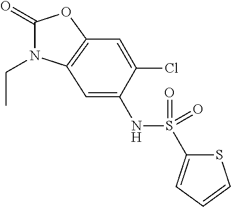 [SMILES: CCn1c(=O)oc2cc(Cl)c(NS(=O)(=O)c3cccs3)cc21] | E |
| 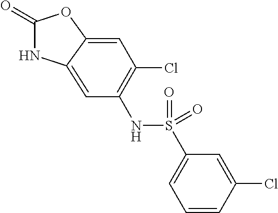 [SMILES: O=c1[nH]c2cc(NS(=O)(=O)c3cccc(Cl)c3)c(Cl)cc2o1] | C |
| 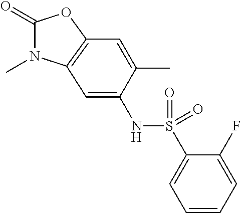 [SMILES: Cc1cc2oc(=O)n(C)c2cc1NS(=O)(=O)c1ccccc1F] | E |
| 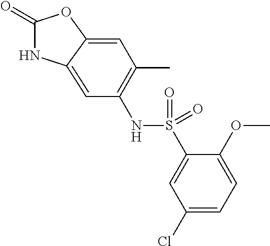 [SMILES: COc1ccc(Cl)cc1S(=O)(=O)Nc1cc2[nH]c(=O)oc2cc1C] | B |
| 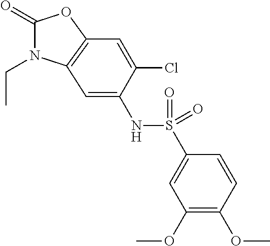 [SMILES: CCn1c(=O)oc2cc(Cl)c(NS(=O)(=O)c3ccc(OC)c(OC)c3)cc21] | E |
| 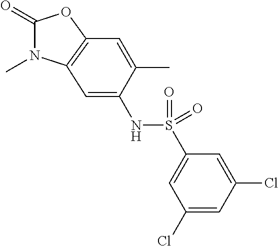 [SMILES: Cc1cc2oc(=O)n(C)c2cc1NS(=O)(=O)c1cc(Cl)cc(Cl)c1] | C |
| 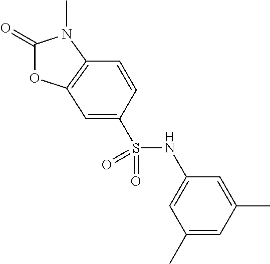 [SMILES: Cc1cc(C)cc(NS(=O)(=O)c2ccc3c(c2)oc(=O)n3C)c1] | A |
| 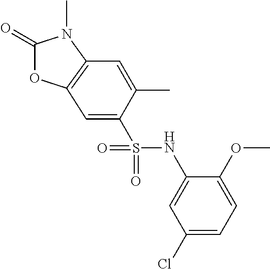 [SMILES: COc1ccc(Cl)cc1NS(=O)(=O)c1cc2oc(=O)n(C)c2cc1C] | B |
| 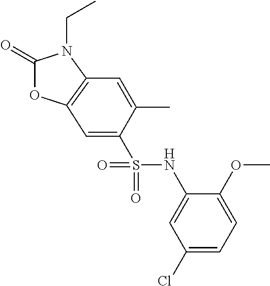 [SMILES: CCn1c(=O)oc2cc(S(=O)(=O)Nc3cc(Cl)ccc3OC)c(C)cc21] | B |
| 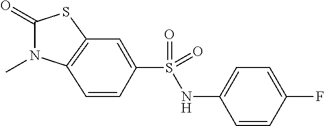 [SMILES: Cn1c(=O)sc2cc(S(=O)(=O)Nc3ccc(F)cc3)ccc21] | A |
| 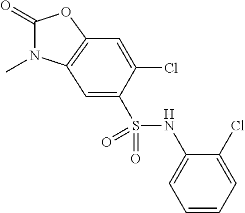 [SMILES: Cn1c(=O)oc2cc(Cl)c(S(=O)(=O)Nc3ccccc3Cl)cc21] | B |
| 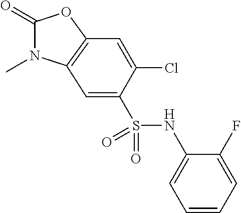 [SMILES: Cn1c(=O)oc2cc(Cl)c(S(=O)(=O)Nc3ccccc3F)cc21] | B |
| 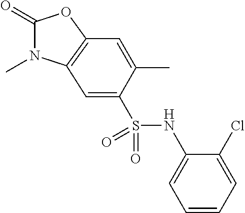 [SMILES: Cc1cc2oc(=O)n(C)c2cc1S(=O)(=O)Nc1ccccc1Cl] | B |
| 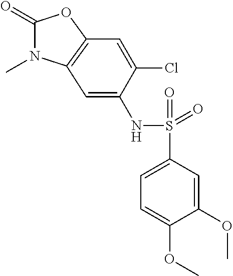 [SMILES: COc1ccc(S(=O)(=O)Nc2cc3c(cc2Cl)oc(=O)n3C)cc1OC] | C |
| 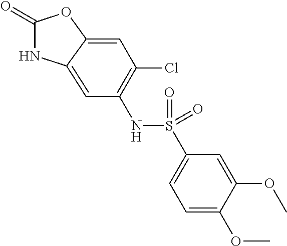 [SMILES: COc1ccc(S(=O)(=O)Nc2cc3[nH]c(=O)oc3cc2Cl)cc1OC] | D |
| 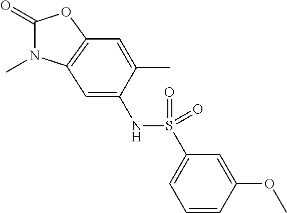 [SMILES: COc1cccc(S(=O)(=O)Nc2cc3c(cc2C)oc(=O)n3C)c1] | C |
| 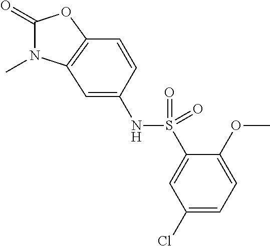 [SMILES: COc1ccc(Cl)cc1S(=O)(=O)Nc1ccc2oc(=O)n(C)c2c1] | A |
| 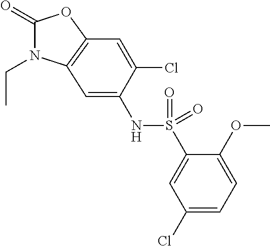 [SMILES: CCn1c(=O)oc2cc(Cl)c(NS(=O)(=O)c3cc(Cl)ccc3OC)cc21] | E |
| 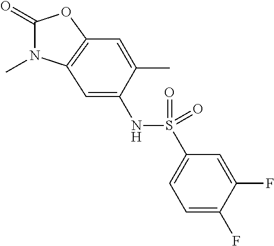 [SMILES: Cc1cc2oc(=O)n(C)c2cc1NS(=O)(=O)c1ccc(F)c(F)c1] | D |
| 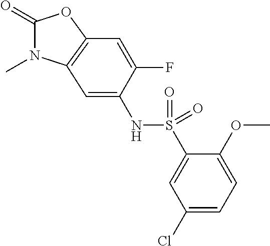 [SMILES: COc1ccc(Cl)cc1S(=O)(=O)Nc1cc2c(cc1F)oc(=O)n2C] | C |
| 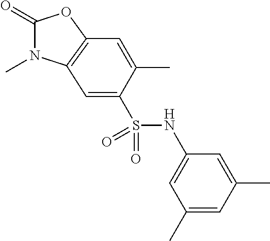 [SMILES: Cc1cc(C)cc(NS(=O)(=O)c2cc3c(cc2C)oc(=O)n3C)c1] | A |
| 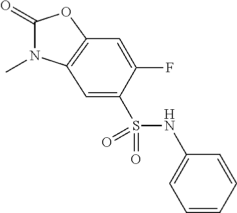 [SMILES: Cn1c(=O)oc2cc(F)c(S(=O)(=O)Nc3ccccc3)cc21] | A |
| 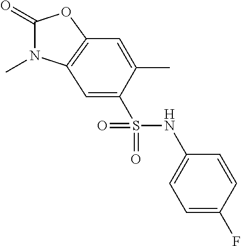 [SMILES: Cc1cc2oc(=O)n(C)c2cc1S(=O)(=O)Nc1ccc(F)cc1] | A |
| 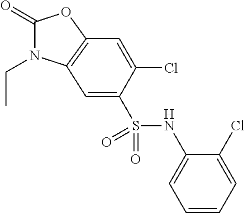 [SMILES: CCn1c(=O)oc2cc(Cl)c(S(=O)(=O)Nc3ccccc3Cl)cc21] | D |
| 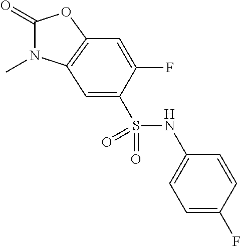 [SMILES: Cn1c(=O)oc2cc(F)c(S(=O)(=O)Nc3ccc(F)cc3)cc21] | B |
| 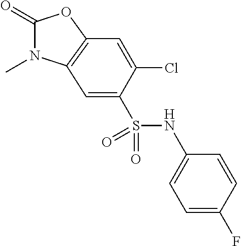 [SMILES: Cn1c(=O)oc2cc(Cl)c(S(=O)(=O)Nc3ccc(F)cc3)cc21] | B |
| 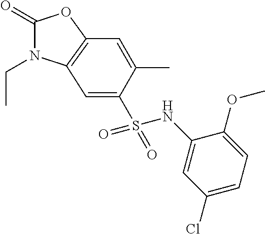 [SMILES: CCn1c(=O)oc2cc(C)c(S(=O)(=O)Nc3cc(Cl)ccc3OC)cc21] | C |
| 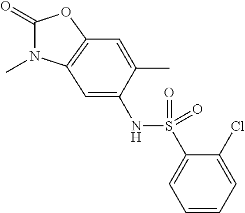 [SMILES: Cc1cc2oc(=O)n(C)c2cc1NS(=O)(=O)c1ccccc1Cl] | C |
| 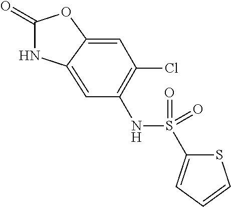 [SMILES: O=c1[nH]c2cc(NS(=O)(=O)c3cccs3)c(Cl)cc2o1] | B |
| 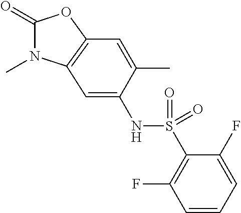 [SMILES: Cc1cc2oc(=O)n(C)c2cc1NS(=O)(=O)c1c(F)cccc1F] | D |
| 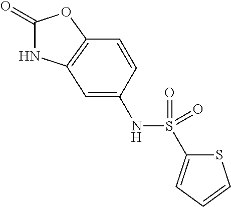 [SMILES: O=c1[nH]c2cc(NS(=O)(=O)c3cccs3)ccc2o1] | C |
| 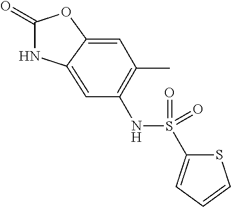 [SMILES: Cc1cc2oc(=O)[nH]c2cc1NS(=O)(=O)c1cccs1] | E |
| 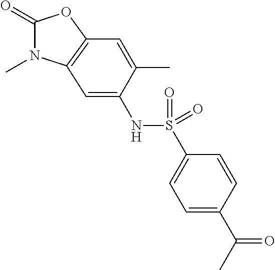 [SMILES: CC(=O)c1ccc(S(=O)(=O)Nc2cc3c(cc2C)oc(=O)n3C)cc1] | C |
| 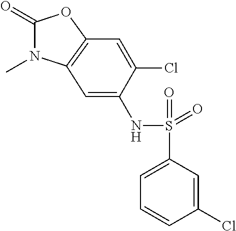 [SMILES: Cn1c(=O)oc2cc(Cl)c(NS(=O)(=O)c3cccc(Cl)c3)cc21] | C |
| 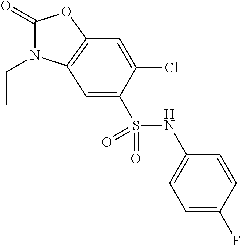 [SMILES: CCn1c(=O)oc2cc(Cl)c(S(=O)(=O)Nc3ccc(F)cc3)cc21] | C |
| 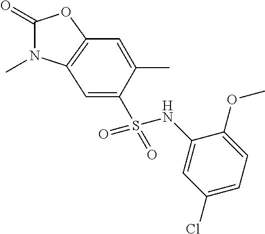 [SMILES: COc1ccc(Cl)cc1NS(=O)(=O)c1cc2c(cc1C)oc(=O)n2C] | B |
| 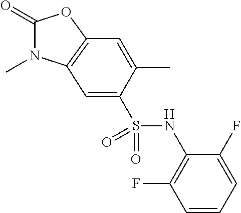 [SMILES: Cc1cc2oc(=O)n(C)c2cc1S(=O)(=O)Nc1c(F)cccc1F] | D |
| 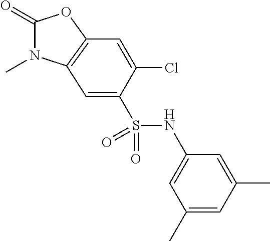 [SMILES: Cc1cc(C)cc(NS(=O)(=O)c2cc3c(cc2Cl)oc(=O)n3C)c1] | A |
| 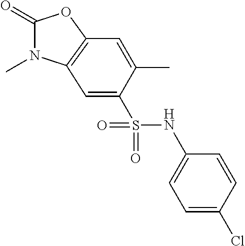 [SMILES: Cc1cc2oc(=O)n(C)c2cc1S(=O)(=O)Nc1ccc(Cl)cc1] | A |
| 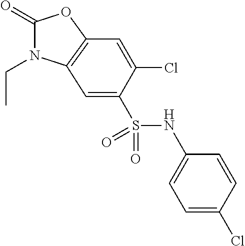 [SMILES: CCn1c(=O)oc2cc(Cl)c(S(=O)(=O)Nc3ccc(Cl)cc3)cc21] | B |
| 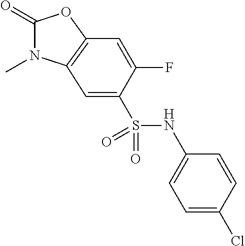 [SMILES: Cn1c(=O)oc2cc(F)c(S(=O)(=O)Nc3ccc(Cl)cc3)cc21] | B |
| 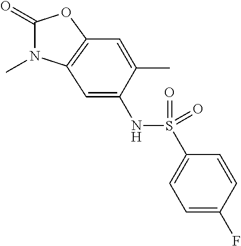 [SMILES: Cc1cc2oc(=O)n(C)c2cc1NS(=O)(=O)c1ccc(F)cc1] | C |
| 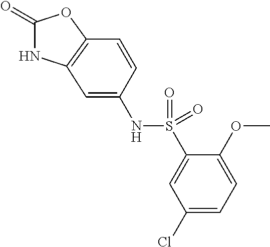 [SMILES: COc1ccc(Cl)cc1S(=O)(=O)Nc1ccc2oc(=O)[nH]c2c1] | C |
| 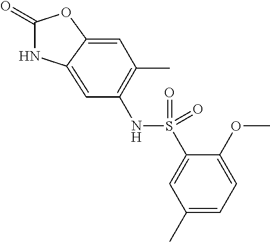 [SMILES: COc1ccc(C)cc1S(=O)(=O)Nc1cc2[nH]c(=O)oc2cc1C] | C |
| 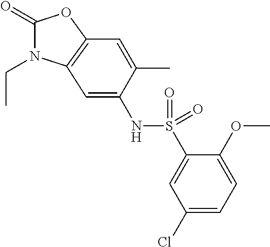 [SMILES: CCn1c(=O)oc2cc(C)c(NS(=O)(=O)c3cc(Cl)ccc3OC)cc21] | C |
| 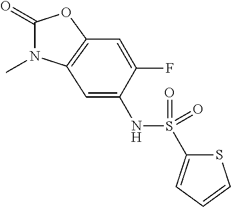 [SMILES: Cn1c(=O)oc2cc(F)c(NS(=O)(=O)c3cccs3)cc21] | D |
| 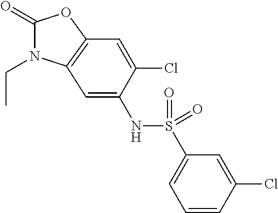 [SMILES: CCn1c(=O)oc2cc(Cl)c(NS(=O)(=O)c3cccc(Cl)c3)cc21] | E |
| 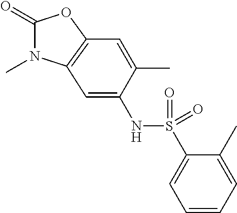 [SMILES: Cc1cc2oc(=O)n(C)c2cc1NS(=O)(=O)c1ccccc1C] | B |
| 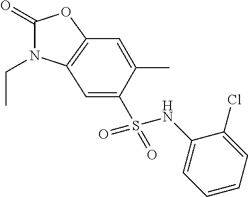 [SMILES: CCn1c(=O)oc2cc(C)c(S(=O)(=O)Nc3ccccc3Cl)cc21] | C |
| 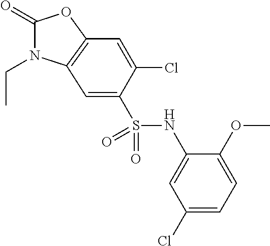 [SMILES: CCn1c(=O)oc2cc(Cl)c(S(=O)(=O)Nc3cc(Cl)ccc3OC)cc21] | E |
| 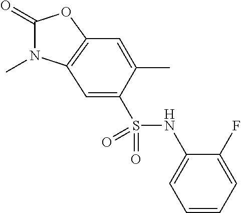 [SMILES: Cc1cc2oc(=O)n(C)c2cc1S(=O)(=O)Nc1ccccc1F] | B |
| 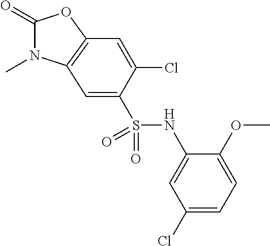 [SMILES: COc1ccc(Cl)cc1NS(=O)(=O)c1cc2c(cc1Cl)oc(=O)n2C] | B |
| 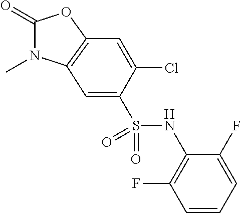 [SMILES: Cn1c(=O)oc2cc(Cl)c(S(=O)(=O)Nc3c(F)cccc3F)cc21] | E |
| 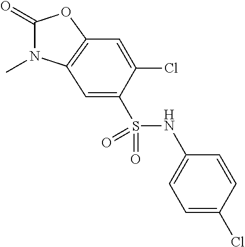 [SMILES: Cn1c(=O)oc2cc(Cl)c(S(=O)(=O)Nc3ccc(Cl)cc3)cc21] | C |
| 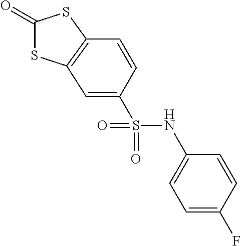 [SMILES: O=c1sc2ccc(S(=O)(=O)Nc3ccc(F)cc3)cc2s1] | C |
| 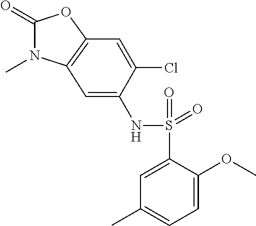 [SMILES: COc1ccc(C)cc1S(=O)(=O)Nc1cc2c(cc1Cl)oc(=O)n2C] | B |
| 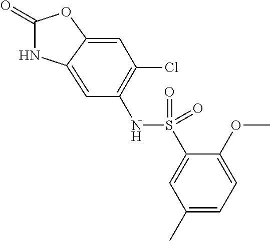 [SMILES: COc1ccc(C)cc1S(=O)(=O)Nc1cc2[nH]c(=O)oc2cc1Cl] | E |
| 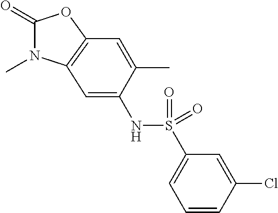 [SMILES: Cc1cc2oc(=O)n(C)c2cc1NS(=O)(=O)c1cccc(Cl)c1] | C |
| 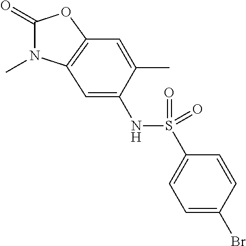 [SMILES: Cc1cc2oc(=O)n(C)c2cc1NS(=O)(=O)c1ccc(Br)cc1] | E |
| 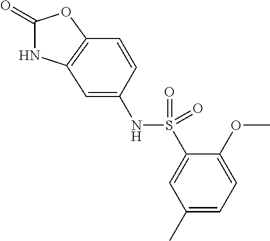 [SMILES: COc1ccc(C)cc1S(=O)(=O)Nc1ccc2oc(=O)[nH]c2c1] | C |
| 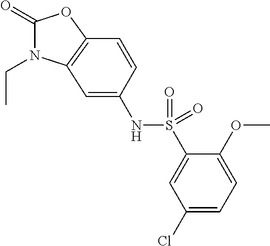 [SMILES: CCn1c(=O)oc2ccc(NS(=O)(=O)c3cc(Cl)ccc3OC)cc21] | E |
| 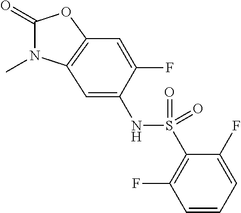 [SMILES: Cn1c(=O)oc2cc(F)c(NS(=O)(=O)c3c(F)cccc3F)cc21] | D |
| 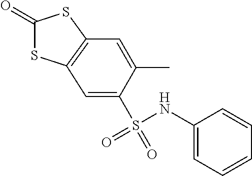 [SMILES: Cc1cc2sc(=O)sc2cc1S(=O)(=O)Nc1ccccc1] | A |
| 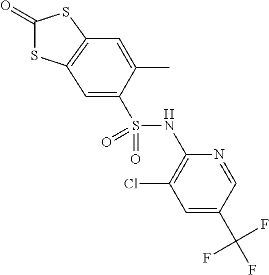 [SMILES: Cc1cc2sc(=O)sc2cc1S(=O)(=O)Nc1ncc(C(F)(F)F)cc1Cl] | B |
| 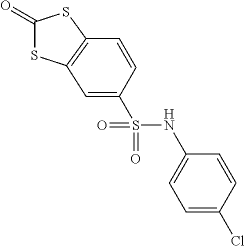 [SMILES: O=c1sc2ccc(S(=O)(=O)Nc3ccc(Cl)cc3)cc2s1] | B |
| 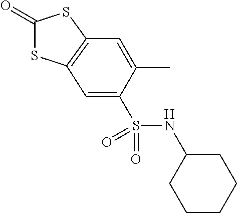 [SMILES: Cc1cc2sc(=O)sc2cc1S(=O)(=O)NC1CCCCC1] | C |
| 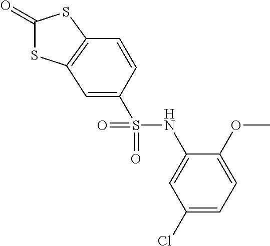 [SMILES: COc1ccc(Cl)cc1NS(=O)(=O)c1ccc2sc(=O)sc2c1] | C |
| 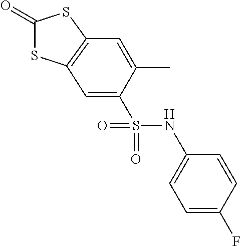 [SMILES: Cc1cc2sc(=O)sc2cc1S(=O)(=O)Nc1ccc(F)cc1] | B |
| 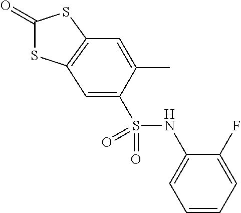 [SMILES: Cc1cc2sc(=O)sc2cc1S(=O)(=O)Nc1ccccc1F] | C |
| 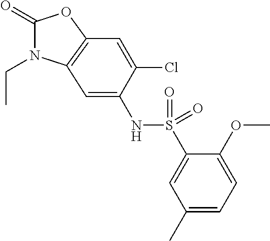 [SMILES: CCn1c(=O)oc2cc(Cl)c(NS(=O)(=O)c3cc(C)ccc3OC)cc21] | E |
| 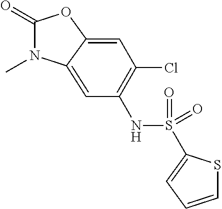 [SMILES: Cn1c(=O)oc2cc(Cl)c(NS(=O)(=O)c3cccs3)cc21] | E |
| 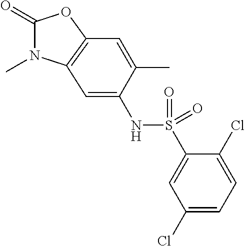 [SMILES: Cc1cc2oc(=O)n(C)c2cc1NS(=O)(=O)c1cc(Cl)ccc1Cl] | C |
| 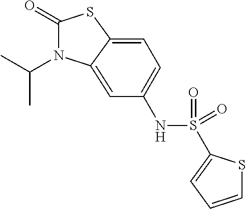 [SMILES: CC(C)n1c(=O)sc2ccc(NS(=O)(=O)c3cccs3)cc21] | E |
| 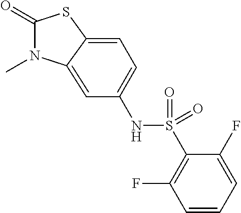 [SMILES: Cn1c(=O)sc2ccc(NS(=O)(=O)c3c(F)cccc3F)cc21] | B |
| 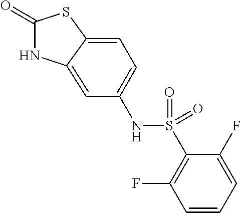 [SMILES: O=c1[nH]c2cc(NS(=O)(=O)c3c(F)cccc3F)ccc2s1] | C |
| 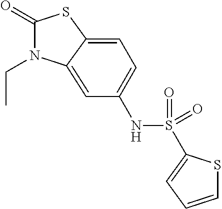 [SMILES: CCn1c(=O)sc2ccc(NS(=O)(=O)c3cccs3)cc21] | C |
| 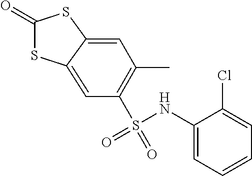 [SMILES: Cc1cc2sc(=O)sc2cc1S(=O)(=O)Nc1ccccc1Cl] | B |
| 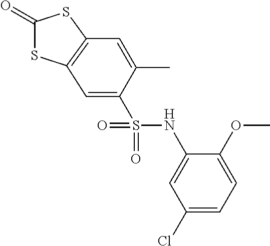 [SMILES: COc1ccc(Cl)cc1NS(=O)(=O)c1cc2sc(=O)sc2cc1C] | B |
| 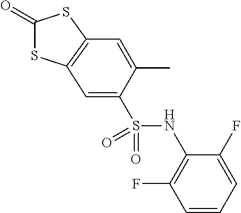 [SMILES: Cc1cc2sc(=O)sc2cc1S(=O)(=O)Nc1c(F)cccc1F] | E |
| 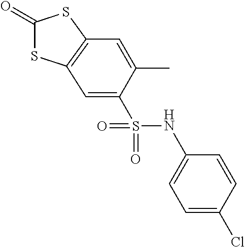 [SMILES: Cc1cc2sc(=O)sc2cc1S(=O)(=O)Nc1ccc(Cl)cc1] | B |
| 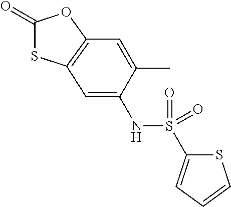 [SMILES: Cc1cc2oc(=O)sc2cc1NS(=O)(=O)c1cccs1] | C |
| 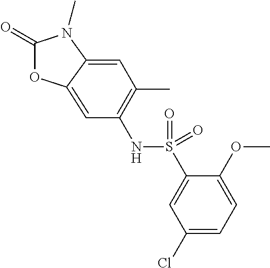 [SMILES: COc1ccc(Cl)cc1S(=O)(=O)Nc1cc2oc(=O)n(C)c2cc1C] | E |
| 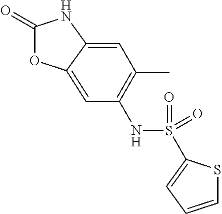 [SMILES: Cc1cc2[nH]c(=O)oc2cc1NS(=O)(=O)c1cccs1] | E |
| 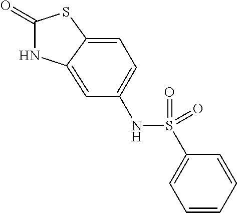 [SMILES: O=c1[nH]c2cc(NS(=O)(=O)c3ccccc3)ccc2s1] | C |
| 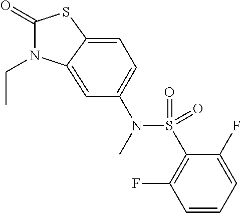 [SMILES: CCn1c(=O)sc2ccc(N(C)S(=O)(=O)c3c(F)cccc3F)cc21] | E |
| 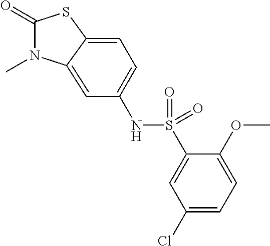 [SMILES: COc1ccc(Cl)cc1S(=O)(=O)Nc1ccc2sc(=O)n(C)c2c1] | B |
| 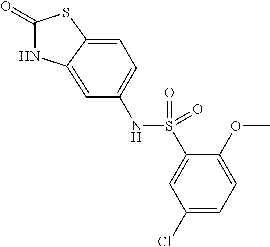 [SMILES: COc1ccc(Cl)cc1S(=O)(=O)Nc1ccc2sc(=O)[nH]c2c1] | B |
| 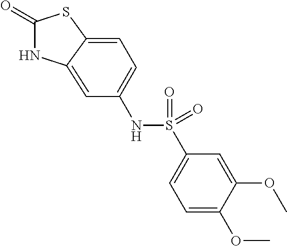 [SMILES: COc1ccc(S(=O)(=O)Nc2ccc3sc(=O)[nH]c3c2)cc1OC] | E |
| 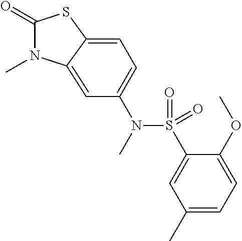 [SMILES: COc1ccc(C)cc1S(=O)(=O)N(C)c1ccc2sc(=O)n(C)c2c1] | E |
| 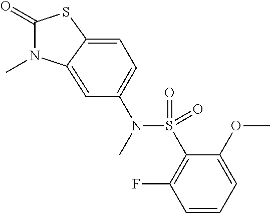 [SMILES: COc1cccc(F)c1S(=O)(=O)N(C)c1ccc2sc(=O)n(C)c2c1] | C |
| 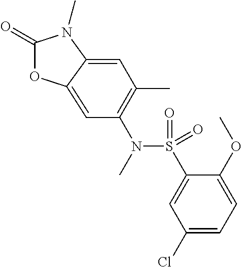 [SMILES: COc1ccc(Cl)cc1S(=O)(=O)N(C)c1cc2oc(=O)n(C)c2cc1C] | E |
| 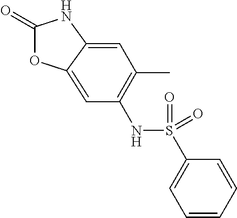 [SMILES: Cc1cc2[nH]c(=O)oc2cc1NS(=O)(=O)c1ccccc1] | E |
| 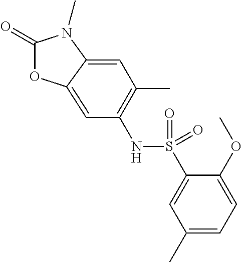 [SMILES: COc1ccc(C)cc1S(=O)(=O)Nc1cc2oc(=O)n(C)c2cc1C] | E |
| 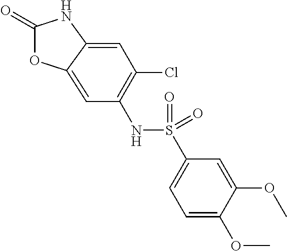 [SMILES: COc1ccc(S(=O)(=O)Nc2cc3oc(=O)[nH]c3cc2Cl)cc1OC] | E |
| 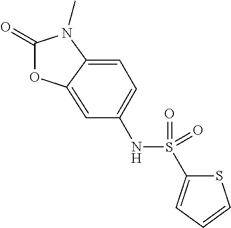 [SMILES: Cn1c(=O)oc2cc(NS(=O)(=O)c3cccs3)ccc21] | C |
| 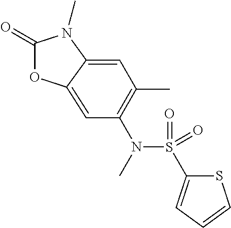 [SMILES: Cc1cc2c(cc1N(C)S(=O)(=O)c1cccs1)oc(=O)n2C] | E |
| 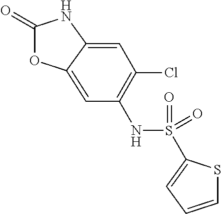 [SMILES: O=c1[nH]c2cc(Cl)c(NS(=O)(=O)c3cccs3)cc2o1] | E |
| 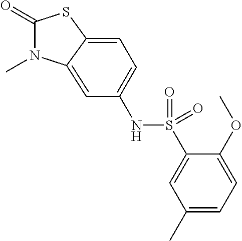 [SMILES: COc1ccc(C)cc1S(=O)(=O)Nc1ccc2sc(=O)n(C)c2c1] | B |
| 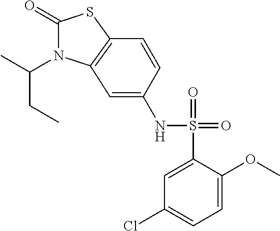 [SMILES: CCC(C)n1c(=O)sc2ccc(NS(=O)(=O)c3cc(Cl)ccc3OC)cc21] | B |
| 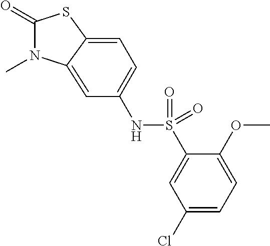 [SMILES: COc1ccc(Cl)cc1S(=O)(=O)Nc1ccc2sc(=O)n(C)c2c1] | C |
| 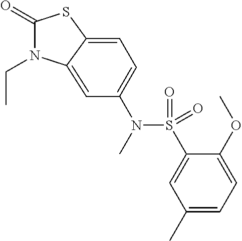 [SMILES: CCn1c(=O)sc2ccc(N(C)S(=O)(=O)c3cc(C)ccc3OC)cc21] | E |
| 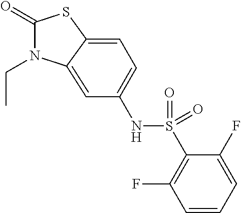 [SMILES: CCn1c(=O)sc2ccc(NS(=O)(=O)c3c(F)cccc3F)cc21] | B |
| 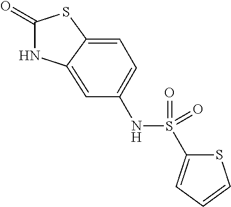 [SMILES: O=c1[nH]c2cc(NS(=O)(=O)c3cccs3)ccc2s1] | C |
| 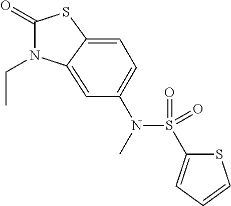 [SMILES: CCn1c(=O)sc2ccc(N(C)S(=O)(=O)c3cccs3)cc21] | E |
| 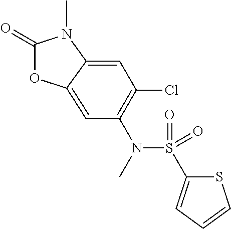 [SMILES: CN(c1cc2oc(=O)n(C)c2cc1Cl)S(=O)(=O)c1cccs1] | E |
| 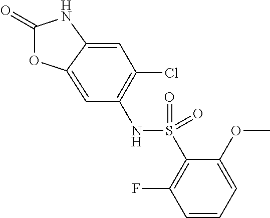 [SMILES: COc1cccc(F)c1S(=O)(=O)Nc1cc2oc(=O)[nH]c2cc1Cl] | E |
| 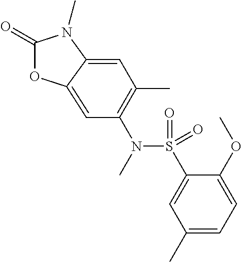 [SMILES: COc1ccc(C)cc1S(=O)(=O)N(C)c1cc2oc(=O)n(C)c2cc1C] | E |
| 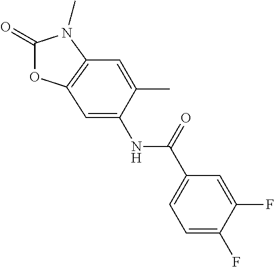 [SMILES: Cc1cc2c(cc1NC(=O)c1ccc(F)c(F)c1)oc(=O)n2C] | E |
| 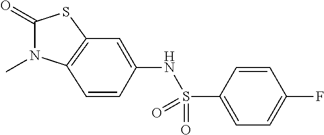 [SMILES: Cn1c(=O)sc2cc(NS(=O)(=O)c3ccc(F)cc3)ccc21] | C |
| 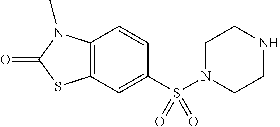 [SMILES: Cn1c(=O)sc2cc(S(=O)(=O)N3CCNCC3)ccc21] | E |
| 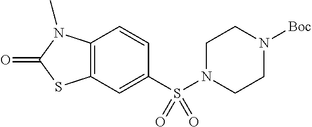 [SMILES: Cn1c(=O)sc2cc(S(=O)(=O)N3CCN(C(=O)OC(C)(C)C)CC3)ccc21] | E |
| 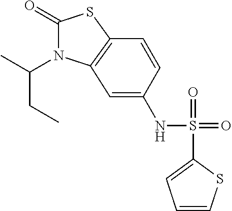 [SMILES: CCC(C)n1c(=O)sc2ccc(NS(=O)(=O)c3cccs3)cc21] | E |
| 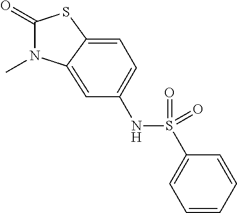 [SMILES: Cn1c(=O)sc2ccc(NS(=O)(=O)c3ccccc3)cc21] | C |
| 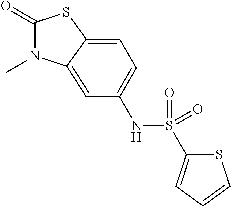 [SMILES: Cn1c(=O)sc2ccc(NS(=O)(=O)c3cccs3)cc21] | C |
| 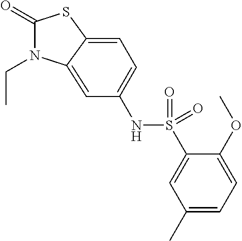 [SMILES: CCn1c(=O)sc2ccc(NS(=O)(=O)c3cc(C)ccc3OC)cc21] | B |
| 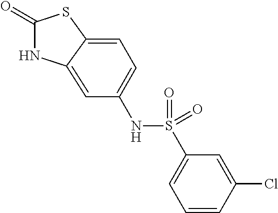 [SMILES: O=c1[nH]c2cc(NS(=O)(=O)c3cccc(Cl)c3)ccc2s1] | C |
| 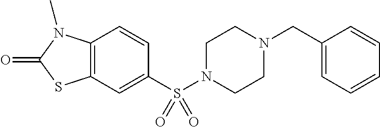 [SMILES: Cn1c(=O)sc2cc(S(=O)(=O)N3CCN(Cc4ccccc4)CC3)ccc21] | E |
| 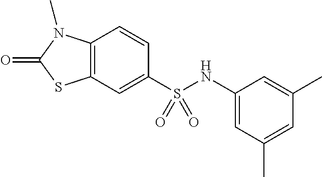 [SMILES: Cc1cc(C)cc(NS(=O)(=O)c2ccc3c(c2)sc(=O)n3C)c1] | A |
| 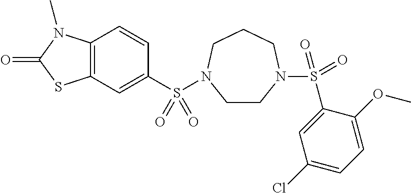 [SMILES: COc1ccc(Cl)cc1S(=O)(=O)N1CCCN(S(=O)(=O)c2ccc3c(c2)sc(=O)n3C)CC1] | A |
| 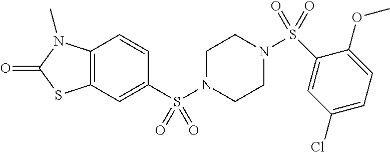 [SMILES: COc1ccc(Cl)cc1S(=O)(=O)N1CCN(S(=O)(=O)c2ccc3c(c2)sc(=O)n3C)CC1] | B |
| 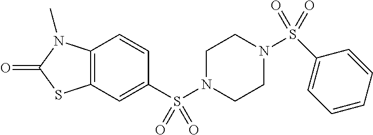 [SMILES: Cn1c(=O)sc2cc(S(=O)(=O)N3CCN(S(=O)(=O)c4ccccc4)CC3)ccc21] | B |
| 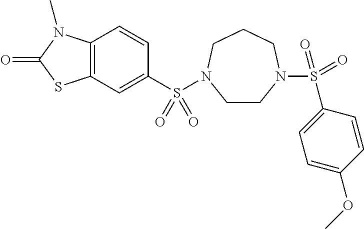 [SMILES: COc1ccc(S(=O)(=O)N2CCCN(S(=O)(=O)c3ccc4c(c3)sc(=O)n4C)CC2)cc1] | B |
| 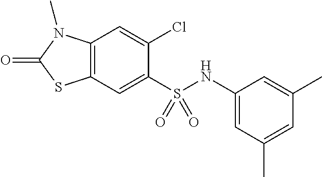 [SMILES: Cc1cc(C)cc(NS(=O)(=O)c2cc3sc(=O)n(C)c3cc2Cl)c1] | A |
| 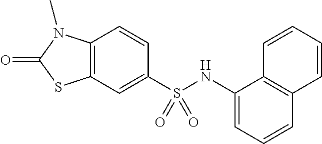 [SMILES: Cn1c(=O)sc2cc(S(=O)(=O)Nc3cccc4ccccc34)ccc21] | B |
| 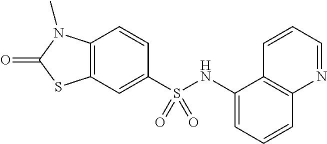 [SMILES: Cn1c(=O)sc2cc(S(=O)(=O)Nc3cccc4ncccc34)ccc21] | B |
| 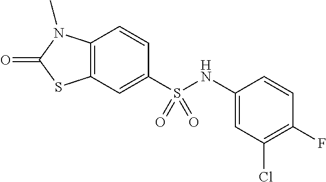 [SMILES: Cn1c(=O)sc2cc(S(=O)(=O)Nc3ccc(F)c(Cl)c3)ccc21] | A |
| 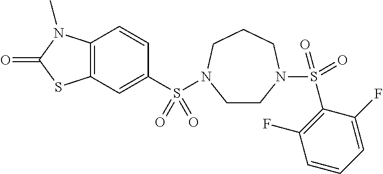 [SMILES: Cn1c(=O)sc2cc(S(=O)(=O)N3CCCN(S(=O)(=O)c4c(F)cccc4F)CC3)ccc21] | B |
| 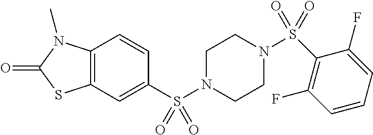 [SMILES: Cn1c(=O)sc2cc(S(=O)(=O)N3CCN(S(=O)(=O)c4c(F)cccc4F)CC3)ccc21] | A |
| 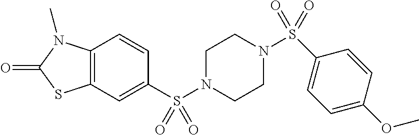 [SMILES: COc1ccc(S(=O)(=O)N2CCN(S(=O)(=O)c3ccc4c(c3)sc(=O)n4C)CC2)cc1] | A |
| 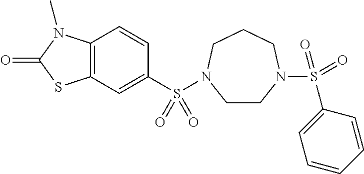 [SMILES: Cn1c(=O)sc2cc(S(=O)(=O)N3CCCN(S(=O)(=O)c4ccccc4)CC3)ccc21] | B |
| 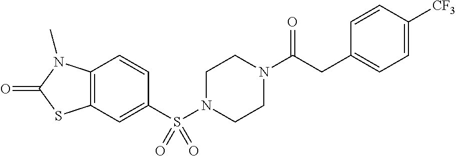 [SMILES: Cn1c(=O)sc2cc(S(=O)(=O)N3CCN(C(=O)Cc4ccc(C(F)(F)F)cc4)CC3)ccc21] | E |
| 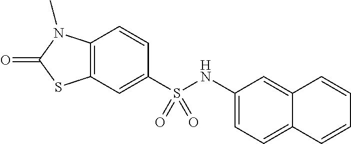 [SMILES: Cn1c(=O)sc2cc(S(=O)(=O)Nc3ccc4ccccc4c3)ccc21] | A |
| 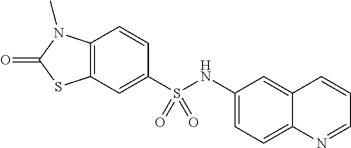 [SMILES: Cn1c(=O)sc2cc(S(=O)(=O)Nc3ccc4ncccc4c3)ccc21] | B |
| 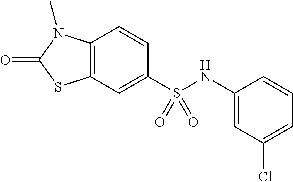 [SMILES: Cn1c(=O)sc2cc(S(=O)(=O)Nc3cccc(Cl)c3)ccc21] | A |
| 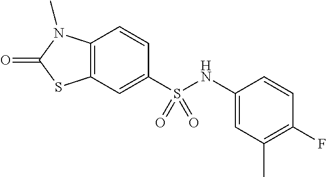 [SMILES: Cc1cc(NS(=O)(=O)c2ccc3c(c2)sc(=O)n3C)ccc1F] | A |
| 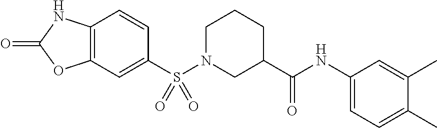 [SMILES: Cc1ccc(NC(=O)C2CCCN(S(=O)(=O)c3ccc4[nH]c(=O)oc4c3)C2)cc1C] | E |
| 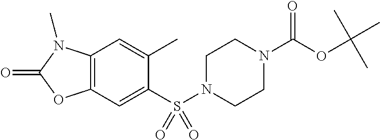 [SMILES: Cc1cc2c(cc1S(=O)(=O)N1CCN(C(=O)OC(C)(C)C)CC1)oc(=O)n2C] | E |
| 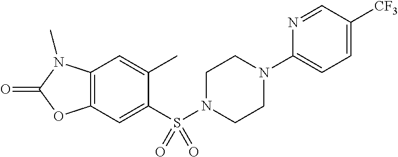 [SMILES: Cc1cc2c(cc1S(=O)(=O)N1CCN(c3ccc(C(F)(F)F)cn3)CC1)oc(=O)n2C] | B |
| 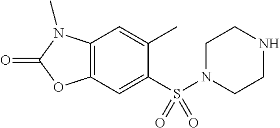 [SMILES: Cc1cc2c(cc1S(=O)(=O)N1CCNCC1)oc(=O)n2C] | E |
| 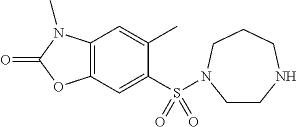 [SMILES: Cc1cc2c(cc1S(=O)(=O)N1CCCNCC1)oc(=O)n2C] | E |
| 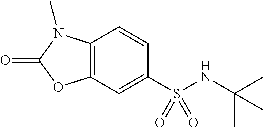 [SMILES: Cn1c(=O)oc2cc(S(=O)(=O)NC(C)(C)C)ccc21] | D |
| 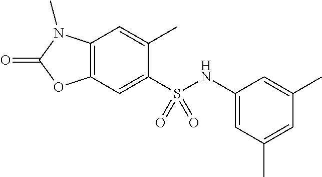 [SMILES: Cc1cc(C)cc(NS(=O)(=O)c2cc3oc(=O)n(C)c3cc2C)c1] | A |
| 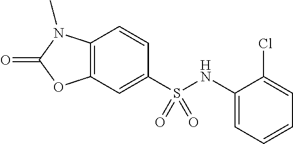 [SMILES: Cn1c(=O)oc2cc(S(=O)(=O)Nc3ccccc3Cl)ccc21] | B |
| 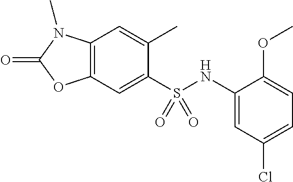 [SMILES: COc1ccc(Cl)cc1NS(=O)(=O)c1cc2oc(=O)n(C)c2cc1C] | B |
| 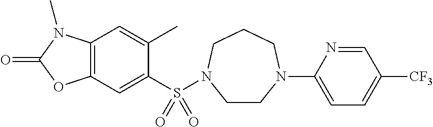 [SMILES: Cc1cc2c(cc1S(=O)(=O)N1CCCN(c3ccc(C(F)(F)F)cn3)CC1)oc(=O)n2C] | B |
| 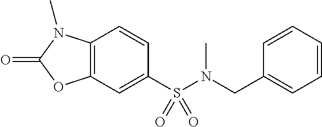 [SMILES: CN(Cc1ccccc1)S(=O)(=O)c1ccc2c(c1)oc(=O)n2C] | B |
| 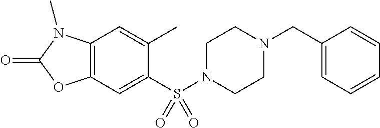 [SMILES: Cc1cc2c(cc1S(=O)(=O)N1CCN(Cc3ccccc3)CC1)oc(=O)n2C] | B |
| 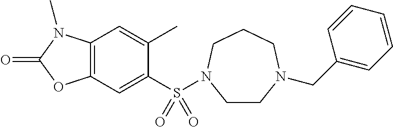 [SMILES: Cc1cc2c(cc1S(=O)(=O)N1CCCN(Cc3ccccc3)CC1)oc(=O)n2C] | E |
| 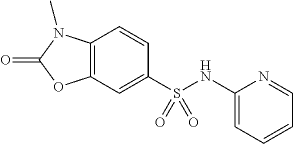 [SMILES: Cn1c(=O)oc2cc(S(=O)(=O)Nc3ccccn3)ccc21] | C |
| 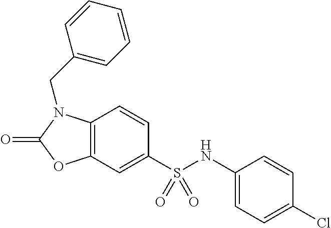 [SMILES: O=c1oc2cc(S(=O)(=O)Nc3ccc(Cl)cc3)ccc2n1Cc1ccccc1] | B |
| 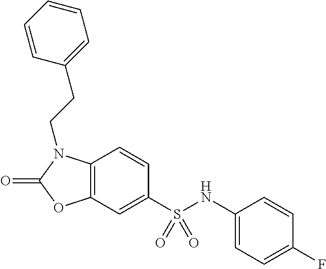 [SMILES: O=c1oc2cc(S(=O)(=O)Nc3ccc(F)cc3)ccc2n1CCc1ccccc1] | B |
| 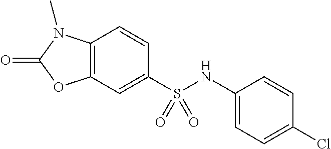 [SMILES: Cn1c(=O)oc2cc(S(=O)(=O)Nc3ccc(Cl)cc3)ccc21] | B |
| 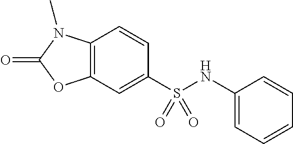 [SMILES: Cn1c(=O)oc2cc(S(=O)(=O)Nc3ccccc3)ccc21] | B |
| 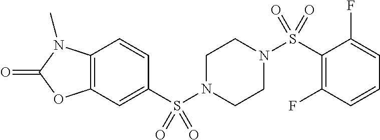 [SMILES: Cn1c(=O)oc2cc(S(=O)(=O)N3CCN(S(=O)(=O)c4c(F)cccc4F)CC3)ccc21] | B |
| 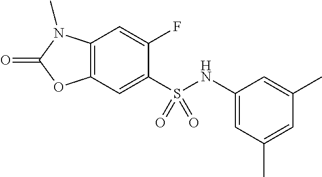 [SMILES: Cc1cc(C)cc(NS(=O)(=O)c2cc3oc(=O)n(C)c3cc2F)c1] | A |
| 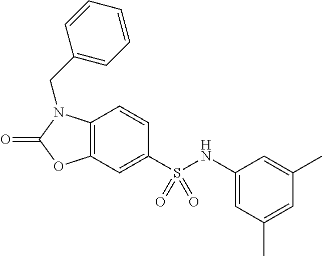 [SMILES: Cc1cc(C)cc(NS(=O)(=O)c2ccc3c(c2)oc(=O)n3Cc2ccccc2)c1] | B |
| 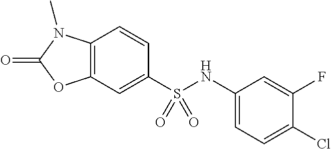 [SMILES: Cn1c(=O)oc2cc(S(=O)(=O)Nc3ccc(Cl)c(F)c3)ccc21] | B |
| 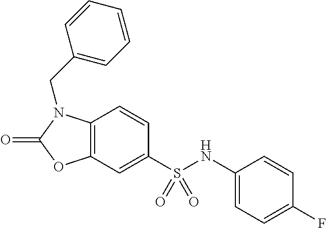 [SMILES: O=c1oc2cc(S(=O)(=O)Nc3ccc(F)cc3)ccc2n1Cc1ccccc1] | B |
| 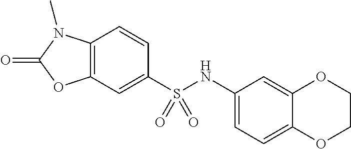 [SMILES: Cn1c(=O)oc2cc(S(=O)(=O)Nc3ccc4c(c3)OCCO4)ccc21] | B |
| 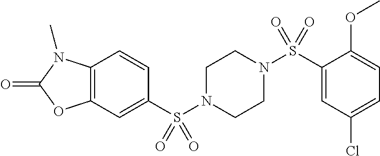 [SMILES: COc1ccc(Cl)cc1S(=O)(=O)N1CCN(S(=O)(=O)c2ccc3c(c2)oc(=O)n3C)CC1] | B |
| 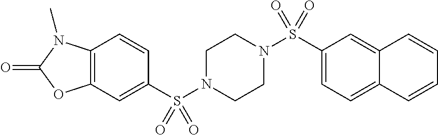 [SMILES: Cn1c(=O)oc2cc(S(=O)(=O)N3CCN(S(=O)(=O)c4ccc5ccccc5c4)CC3)ccc21] | A |
| 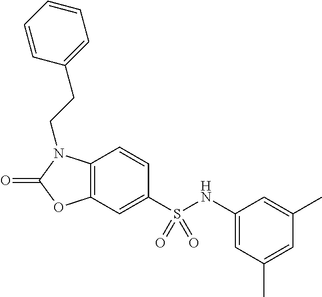 [SMILES: Cc1cc(C)cc(NS(=O)(=O)c2ccc3c(c2)oc(=O)n3CCc2ccccc2)c1] | B |
| 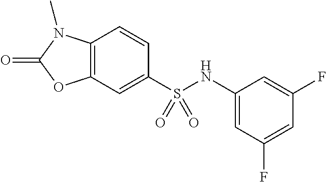 [SMILES: Cn1c(=O)oc2cc(S(=O)(=O)Nc3cc(F)cc(F)c3)ccc21] | B |
| 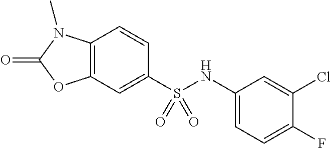 [SMILES: Cn1c(=O)oc2cc(S(=O)(=O)Nc3ccc(F)c(Cl)c3)ccc21] | A |
| 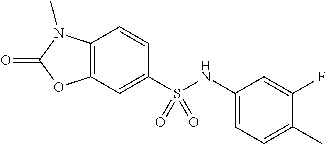 [SMILES: Cc1ccc(NS(=O)(=O)c2ccc3c(c2)oc(=O)n3C)cc1F] | B |
| 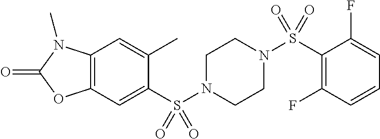 [SMILES: Cc1cc2c(cc1S(=O)(=O)N1CCN(S(=O)(=O)c3c(F)cccc3F)CC1)oc(=O)n2C] | B |
| 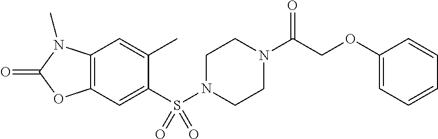 [SMILES: Cc1cc2c(cc1S(=O)(=O)N1CCN(C(=O)COc3ccccc3)CC1)oc(=O)n2C] | D |
| 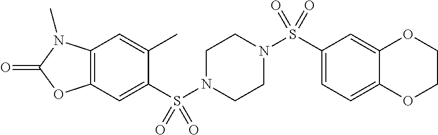 [SMILES: Cc1cc2c(cc1S(=O)(=O)N1CCN(S(=O)(=O)c3ccc4c(c3)OCCO4)CC1)oc(=O)n2C] | A |
| 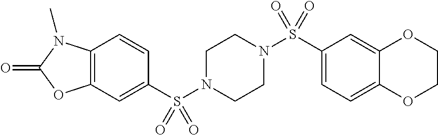 [SMILES: Cn1c(=O)oc2cc(S(=O)(=O)N3CCN(S(=O)(=O)c4ccc5c(c4)OCCO5)CC3)ccc21] | A |
| 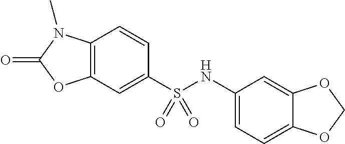 [SMILES: Cn1c(=O)oc2cc(S(=O)(=O)Nc3ccc4c(c3)OCO4)ccc21] | A |
| 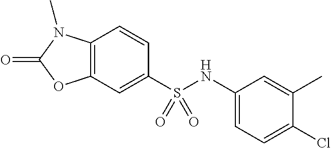 [SMILES: Cc1cc(NS(=O)(=O)c2ccc3c(c2)oc(=O)n3C)ccc1Cl] | A |
| 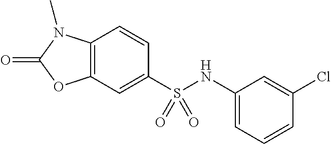 [SMILES: Cn1c(=O)oc2cc(S(=O)(=O)Nc3cccc(Cl)c3)ccc21] | A |
| 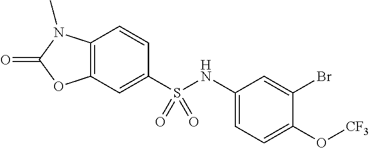 [SMILES: Cn1c(=O)oc2cc(S(=O)(=O)Nc3ccc(OC(F)(F)F)c(Br)c3)ccc21] | B |
| 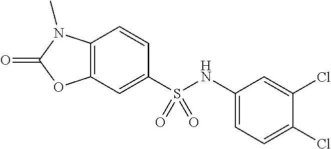 [SMILES: Cn1c(=O)oc2cc(S(=O)(=O)Nc3ccc(Cl)c(Cl)c3)ccc21] | B |
| 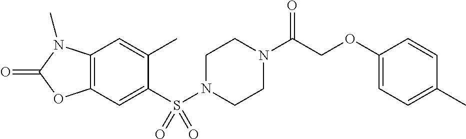 [SMILES: Cc1ccc(OCC(=O)N2CCN(S(=O)(=O)c3cc4oc(=O)n(C)c4cc3C)CC2)cc1] | B |
| 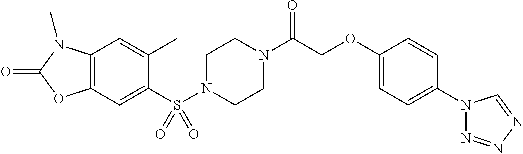 [SMILES: Cc1cc2c(cc1S(=O)(=O)N1CCN(C(=O)COc3ccc(-n4cnnn4)cc3)CC1)oc(=O)n2C] | C |
| 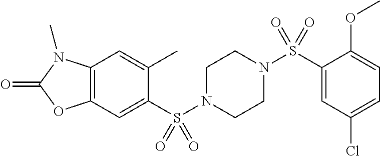 [SMILES: COc1ccc(Cl)cc1S(=O)(=O)N1CCN(S(=O)(=O)c2cc3oc(=O)n(C)c3cc2C)CC1] | E |
| 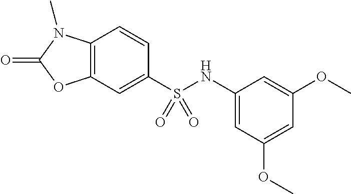 [SMILES: COc1cc(NS(=O)(=O)c2ccc3c(c2)oc(=O)n3C)cc(OC)c1] | A |
| 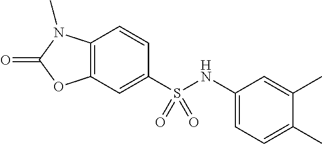 [SMILES: Cc1ccc(NS(=O)(=O)c2ccc3c(c2)oc(=O)n3C)cc1C] | A |
| 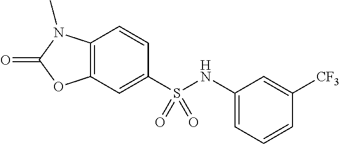 [SMILES: Cn1c(=O)oc2cc(S(=O)(=O)Nc3cccc(C(F)(F)F)c3)ccc21] | B |
| 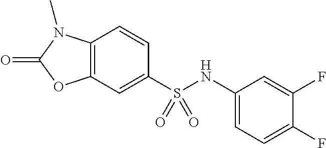 [SMILES: Cn1c(=O)oc2cc(S(=O)(=O)Nc3ccc(F)c(F)c3)ccc21] | A |
| 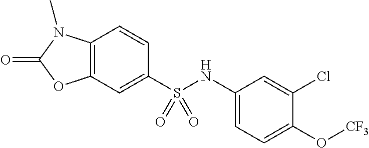 [SMILES: Cn1c(=O)oc2cc(S(=O)(=O)Nc3ccc(OC(F)(F)F)c(Cl)c3)ccc21] | B |
| 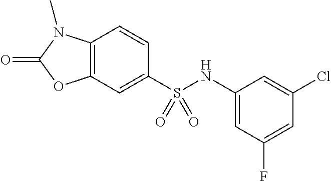 [SMILES: Cn1c(=O)oc2cc(S(=O)(=O)Nc3cc(F)cc(Cl)c3)ccc21] | A |
| 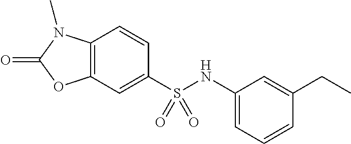 [SMILES: CCc1cccc(NS(=O)(=O)c2ccc3c(c2)oc(=O)n3C)c1] | A |
| 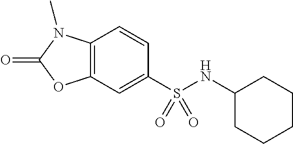 [SMILES: Cn1c(=O)oc2cc(S(=O)(=O)NC3CCCCC3)ccc21] | B |
| 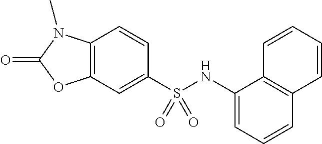 [SMILES: Cn1c(=O)oc2cc(S(=O)(=O)Nc3cccc4ccccc34)ccc21] | B |
| 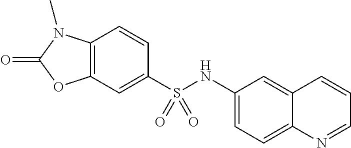 [SMILES: Cn1c(=O)oc2cc(S(=O)(=O)Nc3ccc4ncccc4c3)ccc21] | B |
| 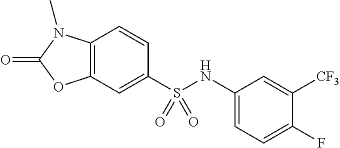 [SMILES: Cn1c(=O)oc2cc(S(=O)(=O)Nc3ccc(F)c(C(F)(F)F)c3)ccc21] | A |
| 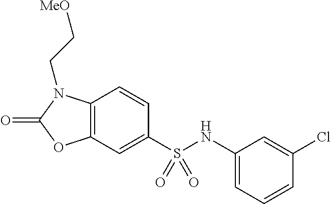 [SMILES: COCCn1c(=O)oc2cc(S(=O)(=O)Nc3cccc(Cl)c3)ccc21] | A |
| 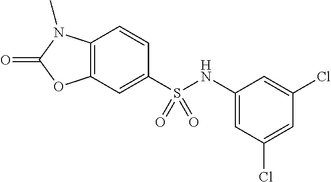 [SMILES: Cn1c(=O)oc2cc(S(=O)(=O)Nc3cc(Cl)cc(Cl)c3)ccc21] | A |
| 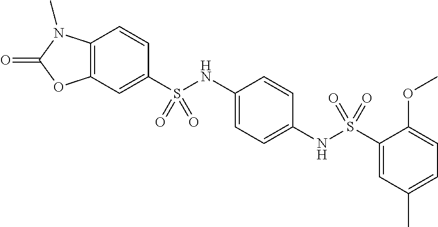 [SMILES: COc1ccc(C)cc1S(=O)(=O)Nc1ccc(NS(=O)(=O)c2ccc3c(c2)oc(=O)n3C)cc1] | B |
| 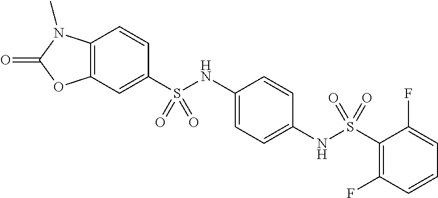 [SMILES: Cn1c(=O)oc2cc(S(=O)(=O)Nc3ccc(NS(=O)(=O)c4c(F)cccc4F)cc3)ccc21] | E |
| 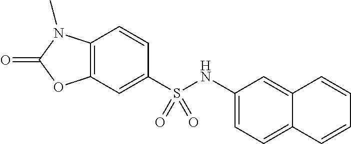 [SMILES: Cn1c(=O)oc2cc(S(=O)(=O)Nc3ccc4ccccc4c3)ccc21] | B |
| 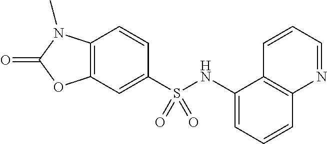 [SMILES: Cn1c(=O)oc2cc(S(=O)(=O)Nc3cccc4ncccc34)ccc21] | C |
| 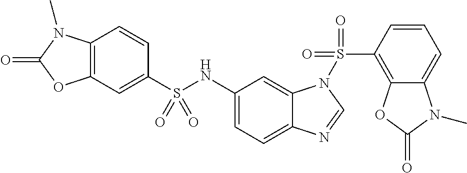 [SMILES: Cn1c(=O)oc2cc(S(=O)(=O)Nc3ccc4ncn(S(=O)(=O)c5cccc6c5oc(=O)n6C)c4c3)ccc21] | B |
| 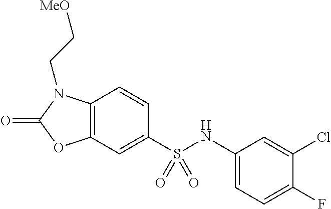 [SMILES: COCCn1c(=O)oc2cc(S(=O)(=O)Nc3ccc(F)c(Cl)c3)ccc21] | A |
| 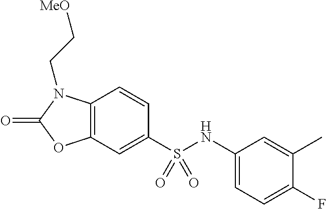 [SMILES: COCCn1c(=O)oc2cc(S(=O)(=O)Nc3ccc(F)c(C)c3)ccc21] | A |
| 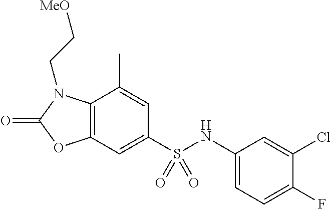 [SMILES: COCCn1c(=O)oc2cc(S(=O)(=O)Nc3ccc(F)c(Cl)c3)cc(C)c21] | A |
| 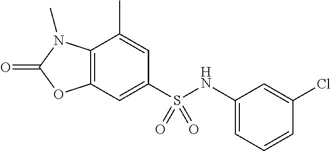 [SMILES: Cc1cc(S(=O)(=O)Nc2cccc(Cl)c2)cc2oc(=O)n(C)c12] | A |
| 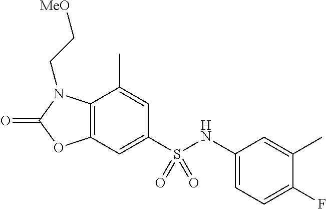 [SMILES: COCCn1c(=O)oc2cc(S(=O)(=O)Nc3ccc(F)c(C)c3)cc(C)c21] | A |
| 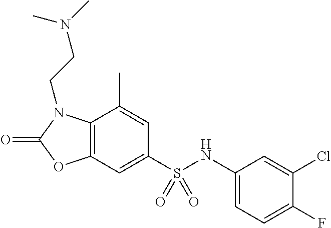 [SMILES: Cc1cc(S(=O)(=O)Nc2ccc(F)c(Cl)c2)cc2oc(=O)n(CCN(C)C)c12] | A |
| 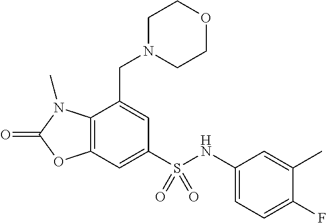 [SMILES: Cc1cc(NS(=O)(=O)c2cc(CN3CCOCC3)c3c(c2)oc(=O)n3C)ccc1F] | B |
| 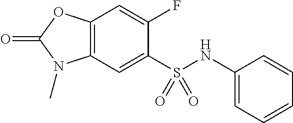 [SMILES: Cn1c(=O)oc2cc(F)c(S(=O)(=O)Nc3ccccc3)cc21] | B |
| 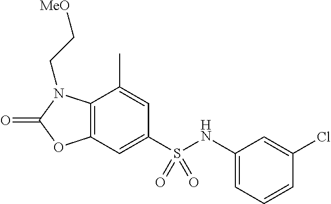 [SMILES: COCCn1c(=O)oc2cc(S(=O)(=O)Nc3cccc(Cl)c3)cc(C)c21] | A |
| 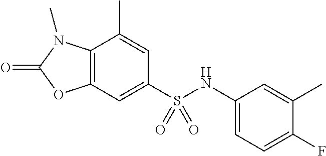 [SMILES: Cc1cc(NS(=O)(=O)c2cc(C)c3c(c2)oc(=O)n3C)ccc1F] | A |
| 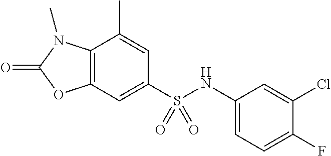 [SMILES: Cc1cc(S(=O)(=O)Nc2ccc(F)c(Cl)c2)cc2oc(=O)n(C)c12] | A |
| 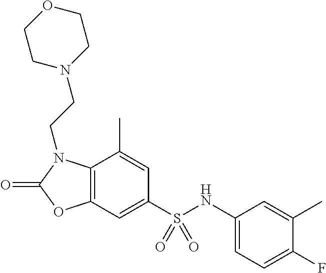 [SMILES: Cc1cc(NS(=O)(=O)c2cc(C)c3c(c2)oc(=O)n3CCN2CCOCC2)ccc1F] | B |
| 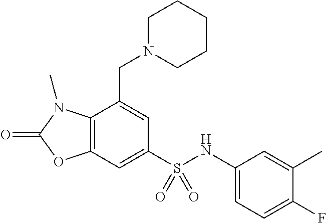 [SMILES: Cc1cc(NS(=O)(=O)c2cc(CN3CCCCC3)c3c(c2)oc(=O)n3C)ccc1F] | B |
| 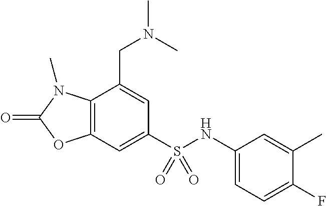 [SMILES: Cc1cc(NS(=O)(=O)c2cc(CN(C)C)c3c(c2)oc(=O)n3C)ccc1F] | D |
| 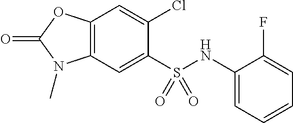 [SMILES: Cn1c(=O)oc2cc(Cl)c(S(=O)(=O)Nc3ccccc3F)cc21] | B |
| 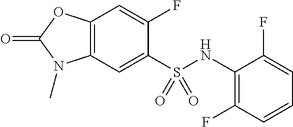 [SMILES: Cn1c(=O)oc2cc(F)c(S(=O)(=O)Nc3c(F)cccc3F)cc21] | D |
| 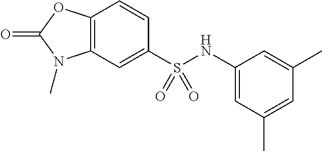 [SMILES: Cc1cc(C)cc(NS(=O)(=O)c2ccc3oc(=O)n(C)c3c2)c1] | A |
| 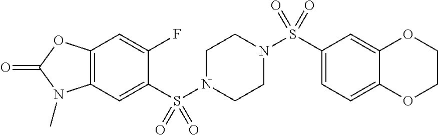 [SMILES: Cn1c(=O)oc2cc(F)c(S(=O)(=O)N3CCN(S(=O)(=O)c4ccc5c(c4)OCCO5)CC3)cc21] | A |
| 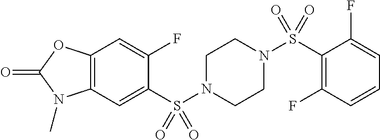 [SMILES: Cn1c(=O)oc2cc(F)c(S(=O)(=O)N3CCN(S(=O)(=O)c4c(F)cccc4F)CC3)cc21] | A |
| 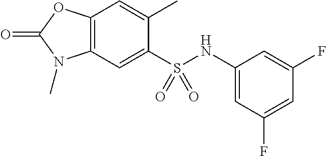 [SMILES: Cc1cc2oc(=O)n(C)c2cc1S(=O)(=O)Nc1cc(F)cc(F)c1] | B |
| 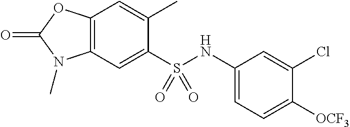 [SMILES: Cc1cc2oc(=O)n(C)c2cc1S(=O)(=O)Nc1ccc(OC(F)(F)F)c(Cl)c1] | B |
| 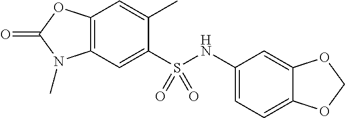 [SMILES: Cc1cc2oc(=O)n(C)c2cc1S(=O)(=O)Nc1ccc2c(c1)OCO2] | A |
| 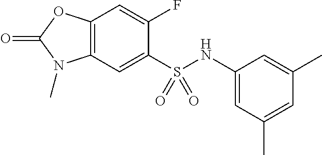 [SMILES: Cc1cc(C)cc(NS(=O)(=O)c2cc3c(cc2F)oc(=O)n3C)c1] | A |
| 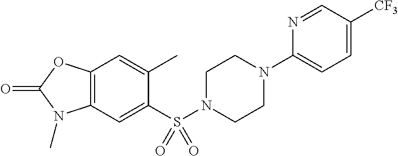 [SMILES: Cc1cc2oc(=O)n(C)c2cc1S(=O)(=O)N1CCN(c2ccc(C(F)(F)F)cn2)CC1] | B |
| 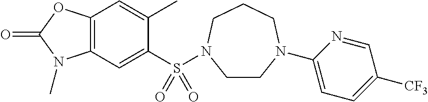 [SMILES: Cc1cc2oc(=O)n(C)c2cc1S(=O)(=O)N1CCCN(c2ccc(C(F)(F)F)cn2)CC1] | B |
| 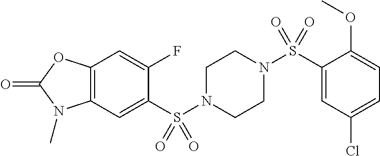 [SMILES: COc1ccc(Cl)cc1S(=O)(=O)N1CCN(S(=O)(=O)c2cc3c(cc2F)oc(=O)n3C)CC1] | A |
| 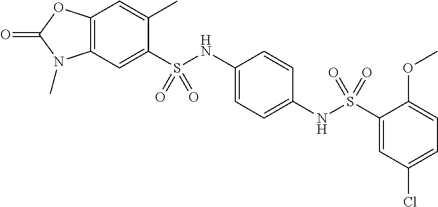 [SMILES: COc1ccc(Cl)cc1S(=O)(=O)Nc1ccc(NS(=O)(=O)c2cc3c(cc2C)oc(=O)n3C)cc1] | D |
| 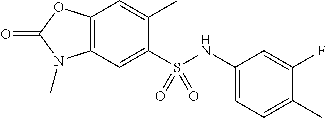 [SMILES: Cc1ccc(NS(=O)(=O)c2cc3c(cc2C)oc(=O)n3C)cc1F] | A |
| 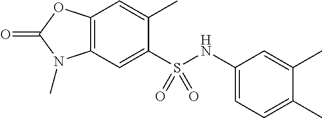 [SMILES: Cc1ccc(NS(=O)(=O)c2cc3c(cc2C)oc(=O)n3C)cc1C] | A |
| 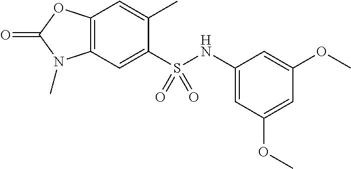 [SMILES: COc1cc(NS(=O)(=O)c2cc3c(cc2C)oc(=O)n3C)cc(OC)c1] | B |
| 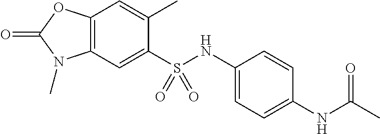 [SMILES: CC(=O)Nc1ccc(NS(=O)(=O)c2cc3c(cc2C)oc(=O)n3C)cc1] | D |
| 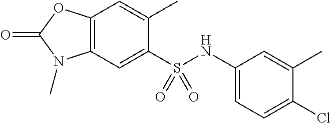 [SMILES: Cc1cc(NS(=O)(=O)c2cc3c(cc2C)oc(=O)n3C)ccc1Cl] | A |
| 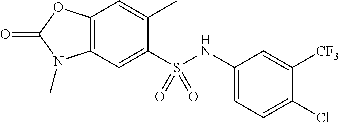 [SMILES: Cc1cc2oc(=O)n(C)c2cc1S(=O)(=O)Nc1ccc(Cl)c(C(F)(F)F)c1] | B |
| 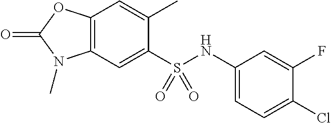 [SMILES: Cc1cc2oc(=O)n(C)c2cc1S(=O)(=O)Nc1ccc(Cl)c(F)c1] | B |
| 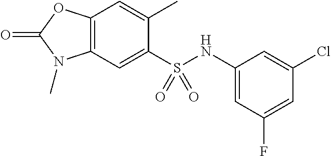 [SMILES: Cc1cc2oc(=O)n(C)c2cc1S(=O)(=O)Nc1cc(F)cc(Cl)c1] | B |
| 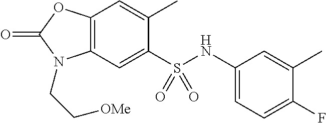 [SMILES: COCCn1c(=O)oc2cc(C)c(S(=O)(=O)Nc3ccc(F)c(C)c3)cc21] | B |
| 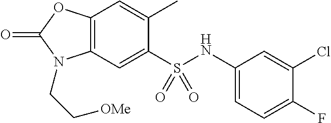 [SMILES: COCCn1c(=O)oc2cc(C)c(S(=O)(=O)Nc3ccc(F)c(Cl)c3)cc21] | B |
| 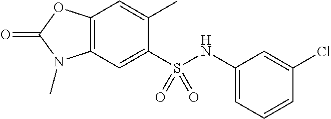 [SMILES: Cc1cc2oc(=O)n(C)c2cc1S(=O)(=O)Nc1cccc(Cl)c1] | A |
| 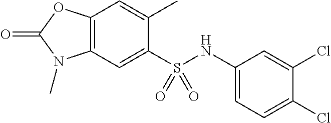 [SMILES: Cc1cc2oc(=O)n(C)c2cc1S(=O)(=O)Nc1ccc(Cl)c(Cl)c1] | B |
| 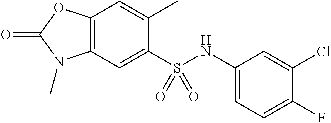 [SMILES: Cc1cc2oc(=O)n(C)c2cc1S(=O)(=O)Nc1ccc(F)c(Cl)c1] | A |
| 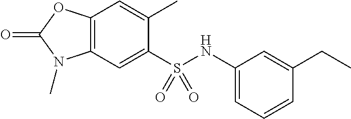 [SMILES: CCc1cccc(NS(=O)(=O)c2cc3c(cc2C)oc(=O)n3C)c1] | A |
| 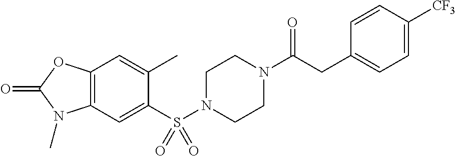 [SMILES: Cc1cc2oc(=O)n(C)c2cc1S(=O)(=O)N1CCN(C(=O)Cc2ccc(C(F)(F)F)cc2)CC1] | D |

The compounds described herein can be made using a variety of synthetic techniques. In some embodiments, a compound described herein may be available from a commercial source. In certain embodiments, the compounds described herein can be made via techniques described in the following applications: U.S. Application No. 61/175,217; U.S. Application No. 61/167,017; U.S. Application No. 61/233,470; U.S. Application No. 61/221,406; U.S. Application No. 61/221,430; and U.S. Application No. 61/292,360.

As can be appreciated by the skilled artisan, methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds) or substituents that can restrict bond rotation, e.g. restriction resulting from the presence of a ring or double bond. Accordingly, all cis/trans and E/Z isomers are expressly included in the present invention.

The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention. Accordingly, as used herein, compounds include polymorphs and hydrates of any particular structural formula.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

The compounds of this invention may be modified by appending appropriate functionalities to enhance selected biological properties, e.g., targeting to a particular tissue. Such modifications are known in the art and include those which increase biological penetration into a given biological compartment (e.g., blood, lymphatic system, central nervous system), increase oral availability, increase solubility to allow administration by injection, alter metabolism and alter rate of excretion.

In an alternate embodiment, the compounds described herein may be used as platforms or scaffolds that may be utilized in combinatorial chemistry techniques for preparation of derivatives and/or chemical libraries of compounds. Such derivatives and libraries of compounds have biological activity and are useful for identifying and designing compounds possessing a particular activity. Combinatorial techniques suitable for utilizing the compounds described herein are known in the art as exemplified by Obrecht, D. and Villalgrodo, J. M., Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, A. W., Curr. Opin. Chem. Bio., (1997) 1, 60. Thus, one embodiment relates to a method of using the compounds described herein for generating derivatives or chemical libraries comprising: 1) providing a body comprising a plurality of wells; 2) providing one or more compounds identified by methods described herein in each well; 3) providing an additional one or more chemicals in each well; 4) isolating the resulting one or more products from each well. An alternate embodiment relates to a method of using the compounds described herein for generating derivatives or chemical libraries comprising: 1) providing one or more compounds described herein attached to a solid support; 2) treating the one or more compounds identified by methods described herein attached to a solid support with one or more additional chemicals; 3) isolating the resulting one or more products from the solid support. In the methods described above, “tags” or identifier or labeling moieties may be attached to and/or detached from the compounds described herein or their derivatives, to facilitate tracking, identification or isolation of the desired products or their intermediates. Such moieties are known in the art. The chemicals used in the aforementioned methods may include, for example, solvents, reagents, catalysts, protecting group and deprotecting group reagents and the like. Examples of such chemicals are those that appear in the various synthetic and protecting group chemistry texts and treatises referenced herein.

Therapeutic Agents.

If desired, additional therapeutic regimens may be provided along with the activators described herein. In some embodiments, the additional therapeutic agent is an inhibitor of cystine oxidation. In some embodiments, the additional therapeutic agent is an inhibitor of glutamine metabolism. For example, therapeutic agents may be administered with the activators of PKM2 activity described herein at concentrations known to be effective for such therapeutic agents. Particularly useful agents include, e.g., chemotherapeutic agents and immunomodulatory agents.

Chemotherapeutic Agents.

Any suitable chemotherapeutic agent may be administered.

Chemotherapeutic agents suitable for the composition described herein include, e.g., asparaginase, bleomycin, busulfan carmustine (BCNU), chlorambucil, cladribine (2-CdA), CPT1 1, cyclophosphamide, cytarabine (Ara-C), dacarbazine, daunorubicin, dexamethasone, doxorubicin (adriamycin), etoposide, fludarabine, 5-fluorouracil (5FU), hydroxyurea, idarubicin, ifosfamide, interferon-α (native or recombinant), levamisole, lomustine (CCNU), mechlorethamine (nitrogen mustard), melphalan, mercaptopurine, methotrexate, mitomycin, mitoxantrone, paclitaxel, pentostatin, prednisone, procarbazine, tamoxifen, taxol-related compounds, 6-thioguanine, topotecan, vinblastine, and vincristine. Exemplary agents include cisplatinum, carboplatinum, oxaliplatinum, and pemetrexed. Exemplary chemotherapeutic agents are listed in, e.g., U.S. Pat. Nos. 6,864,275 and 6,984,654, hereby incorporated by reference. Hormonal therapy can be administered and may include, e.g., anti-estrogens and anti-androgens. Anti-estrogen therapy can be used in the treatment of breast cancer. Anti-androgen therapy can be used in the treatment of prostate cancer.

Immunomodulatory Agents.

Immunomodulatory agents are agents that can elicit or suppress an immune response. Examples of useful immunomodulatory agents include non-steroidal immunophilin-dependent immunosuppressants, e.g., ascomycin, cyclosporine (e.g., Restasis), everolimus, pimecrolimus, rapamycin, and tacrolimus. Also included are steroids, e.g., beclomethasone, budesonide, dexamethasone, fluorometholone, fluticasone, hydrocortisone, loteprednol etabonate, medrysone, rimexolone, and triamcinolone. Exemplary steroids are listed in, e.g., U.S. Pat. Nos. 5,837,698 and 6,909,007, hereby incorporated by reference.

Additional Therapeutic Regimens.

If more than one agent is employed, therapeutic agents may be delivered separately or may be admixed into a single formulation. When agents are present in different pharmaceutical compositions, different routes of administration may be employed. Routes of administration include, e.g., ocular, inhalation, parenteral, dermal, transdermal, buccal, rectal, sublingual, periungual, nasal, topical administration, or oral administration. Parenteral administration includes intravenous, intraperitoneal, subcutaneous, and intramuscular administration.

The therapeutic agents described herein may be admixed with additional active or inert ingredients, e.g., in conventional pharmaceutically acceptable carriers. A pharmaceutical carrier can be any compatible, non-toxic substance suitable for the administration of the compositions of the present invention to a patient. Pharmaceutically acceptable carriers include, for example, water, saline, buffers and other compounds, described, for example, in the Merck Index, Merck & Co., Rahway, N.J. Slow-release formulations or a slow-release apparatus may be also be used for continuous administration.

In addition to the administration of therapeutic agents, the additional therapeutic regimen may involve other therapies, including modification to the lifestyle of the subject being treated.

Formulation of Pharmaceutical Compositions.

The administration of the compositions described herein may be by any suitable means that results in a concentration of the activator and, optionally, therapeutic agent, that is effective in treating the disease associated with PKM2 function and characterized as having abnormally low serine levels.

The composition may be contained in any appropriate amount in any suitable carrier substance. The composition may be provided in a dosage form that is suitable for the oral, parenteral (e.g., intravenous or intramuscular), rectal, cutaneous, nasal, vaginal, inhalant, skin (e.g., a patch), ocular, or intracranial administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions according to the invention may be formulated to release the active agent immediately upon administration or at any predetermined time or time period after administration. The latter types of compositions are generally known as controlled-release formulations, which include (i) formulations that create substantially constant concentrations of a PKM2 activator within the body over an extended period of time; (ii) formulations that after a predetermined lag time create substantially constant concentrations of the PKM2 activator within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the combination to a particular target cell type. Administration of the combination in the form of a controlled-release formulation is especially preferred for compounds having a narrow absorption window in the gastro-intestinal tract or a relatively short biological half-life.

Any of a number of strategies can be pursued in order to obtain controlled release in which the rate of release outweighs the rate of metabolism of the composition in question. In one example, controlled release is obtained by appropriate selection of various formulation parameters and ingredients, including, e.g., various types of controlled release compositions and coatings. Thus, the combination is formulated with appropriate excipients into a pharmaceutical composition that, upon administration, releases the combination in a controlled manner. Examples include single or multiple unit tablet or capsule compositions, oil solutions, suspensions, emulsions, microcapsules, molecular complexes, microspheres, nanoparticles, patches, and liposomes.

Formulations for parenteral administration may, for example, contain excipients, sterile water, or saline, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, and liposomes) may be used to control the biodistribution of the compounds. Other potentially useful parenteral delivery systems include ethylene-vinyl acetate copolymer particles, osmotic pumps, implantable infusion systems, and liposomes.

Formulations for inhalation may contain excipients or may be aqueous solutions containing, for example, polyoxyethylene-9-lauryl ether, glycolate and deoxycholate, or may be oily solutions for administration in the form of nasal drops, or as a gel. The concentration of the compound in the formulation will vary depending upon a number of factors, including the dosage of the drug to be administered, and the route of administration.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc). Formulations for oral use may also be provided in unit dosage form as chewable tablets, tablets, caplets, or capsules (e.g., as hard gelatin capsules wherein the active ingredient is mixed with an inert solid diluent or as soft gelatin capsules wherein the active ingredient is mixed with water or an oil medium).

The composition may be optionally administered as a pharmaceutically acceptable salt, such as, e.g., a non-toxic acid addition salt or metal complex that is commonly used in the pharmaceutical industry. Examples of acid addition salts include, e.g., organic acids (e.g., acetic, lactic, pamoic, maleic, citric, malic, ascorbic, succinic, benzoic, palmitic, suberic, salicylic, tartaric, methanesulfonic, toluenesulfonic, or trifluoroacetic acids), polymeric acids (e.g., tannic acid or carboxymethyl cellulose), and inorganic acids (e.g., hydrochloric acid, hydrobromic acid, sulfuric acid, or phosphoric acid). Metal complexes include, e.g., zinc and iron complexes.

The formulations can be administered to human subjects in therapeutically effective amounts. Typical dose ranges are from about 0.01 μg/kg to about 2 mg/kg of body weight per day. The preferred dosage of drug to be administered is likely to depend on such variables as the type and extent of the disorder, the overall health status of the particular subject, the specific compound being administered, the excipients used to formulate the compound, and its route of administration. Standard clinical trials maybe used to optimize the dose and dosing frequency for any particular composition.

Dosages.

The pharmaceutical compositions described herein may be administered once, twice, three times, four times, or five times each day, or in other quantities and frequencies. Alternatively, the pharmaceutical composition may be administered once per week, twice per week, three times per week, four times per week, five times per week, or six times per week. Therapy with the composition described herein can continue until the disease or disorder has been ameliorated. The duration of therapy can be, e.g., one week to one month; alternatively, the pharmaceutical composition can be administered for a shorter or a longer duration. Continuous daily dosing with the compositions used in the methods and kits described herein may not be required. A therapeutic regimen may require cycles, during which time a composition is not administered, or therapy may be provided on an as-needed basis.

Appropriate dosages of compounds used in the methods described herein depend on several factors, including the administration method, the severity of the disease, and the age, weight, and health of the patient to be treated. Additionally, pharmacogenomic information (e.g., the effect of genotype on the pharmacokinetic, pharmacodynamic, or efficacy profile of a therapeutic) about a particular patient may affect the dosage used.

### EXAMPLE

FIG. 1 shows that an PKM2 activator, AGI-752 has no effect on viability of A549 cells grown in standard media conditions under normoxia or hypoxia, as evaluated by CTG (ATP levels) or cell count. A549 cells were cultured in RPMI medium and 10% FBS (fetal bovine serum). IC50 values>>50 μM.

FIGS. 2A-2D also show that AGI-752 has no effect on viability of A549 cells under various serum/glutamine concentrations. All the experiments in FIGS. 2A-2D were performed in DMEM base media.

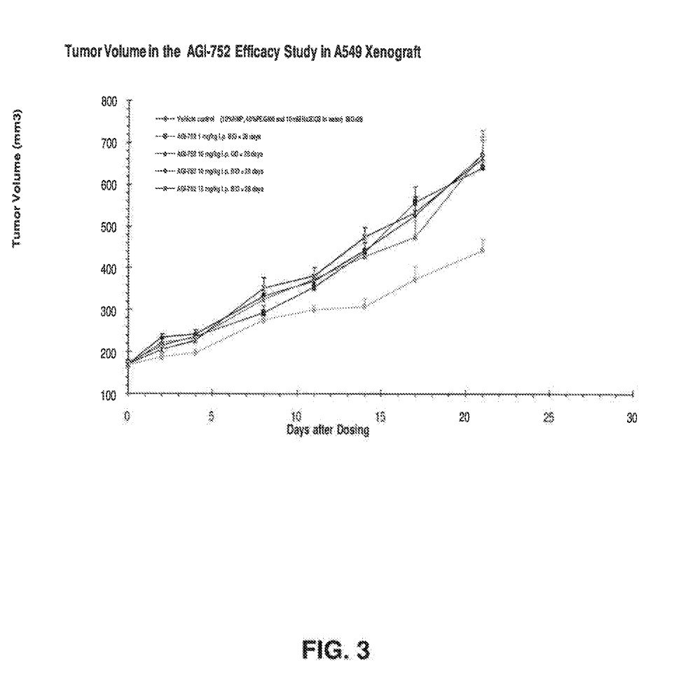

AGI-752 also had no effect on cell viability in a variety of cell lines (including A549, AsPC-1, LOVO, MIA PaCa-2, RPMI 8226, HCT 116, MDA-MB-231, and BT-474), and no effect on cell proliferation in vitro under many cell growth conditions. Multiple cells lines (including A549, 786-0, H460, HCT15, SKMEL28, Calu6, U118, HepG2, LN18, and HEK293) were screened for PKM2 activator growth inhibition. More than 10 growth conditions were assayed, such as by varying FBS, glucose, and glutamine levels; normoxia and hypoxia conditions; soft agar versus plastic substrate; and several PKM2 activators representing three different chemical scaffolds. AGI-752 also had no effect on H460 cell proliferation in a 3D matrigel assay. Despite the above negative results in experiments testing the effects of AGI-752, the compound was found to cause ˜35% tumor growth inhibition when administered at 10 mg/kg BID (twice daily) (FIG. 3). This result suggested that there is some element of the tumor microenvironment that makes tumor cells sensitive to PKM2 activation.

PKM2 activity is directly regulated allosterically by at least four amino acids. The enzyme is activated by serine, and inhibited by phenylalanine, alanine and cysteine. PKM2 also catalyzes the conversion of phosphoenolpyruvate (PEP) to pyruvate, which is connected directly or indirectly to many amino acid biosynthetic pathways (such as the interconversion of alanine to pyruvate). PKM2 also regulates the rate-limiting step in glycolysis, and several glycolytic intermediates are branch points for amino acid biosynthesis (e.g., 3PG to serine).

Experiments were designed to test the effect of amino acid variation on sensitivity of cells in culture to AGI-752. RPMI medium was custom-made with no glucose or amino acids. Dialyzed FBS (dFBS)/glucose/glutamine was added back; BME (Basal Medium Eagle; 12 amino acids (8 essential and 4 conditionally essential)) was optionally added back; NEAA mixture (7 non-essential amino acid mixture (Ala, Gly, Pro, Glu, Asp, Asn, or Ser)) was optionally added back; and sodium pyruvate was optionally added back. AGI-752 (2 μM) was tested for an effect on the cell viability.

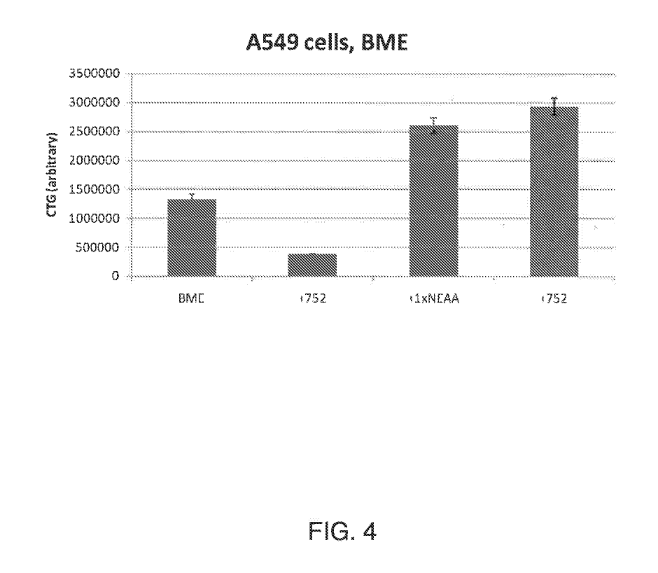

Cells grown in BME medium (12 amino acids) with 3% dialyzed FBS, 5 mM glucose, 0.5 mM to 2 mM glutamine, were sensitive to AGI-752 (FIG. 4). Adding NEAA mixture (7 amino acids) blocked the sensitivity to AGI-752 (BME-NEAA approximates full RPMI media).

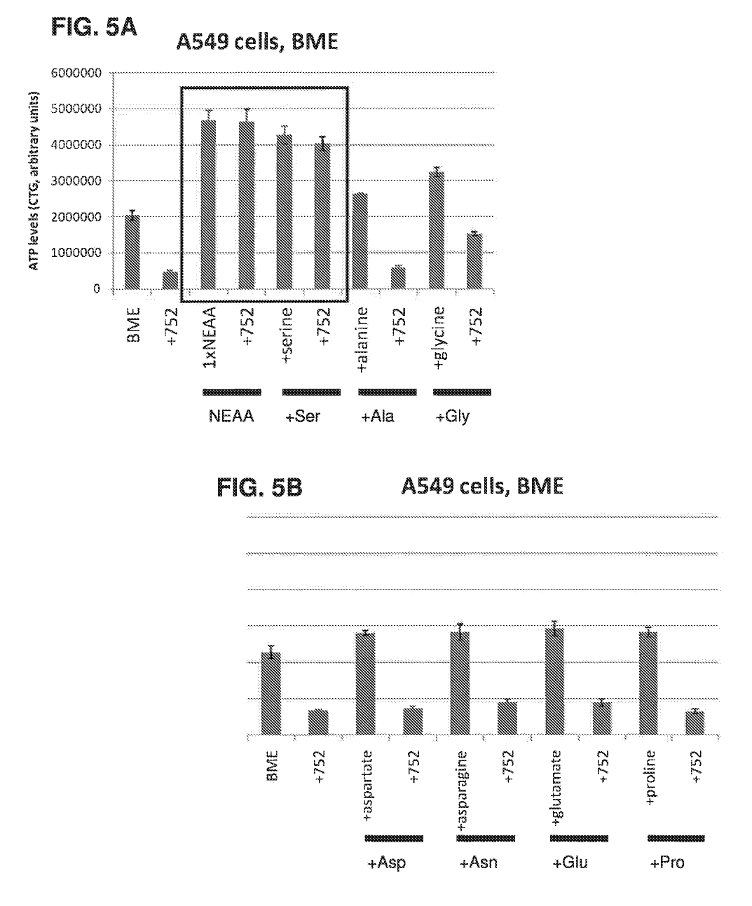

FIGS. 5A and 5B show that serine alone was able to reverse sensitivity to AGI-752 in A549 cells. Cells were grown in BME medium (12 amino acids), and either NEAA mixture (7 amino acids) was added, or each amino acid was added individually. Sensitivity was either reversed by addition of 1×NEAA or serine alone, but not by the addition of any other amino acid alone. A partial reversal of the effect was observed with glycine. Notably, serine is converted to glycine by SHMT (serine hydroxymethyltransferase).

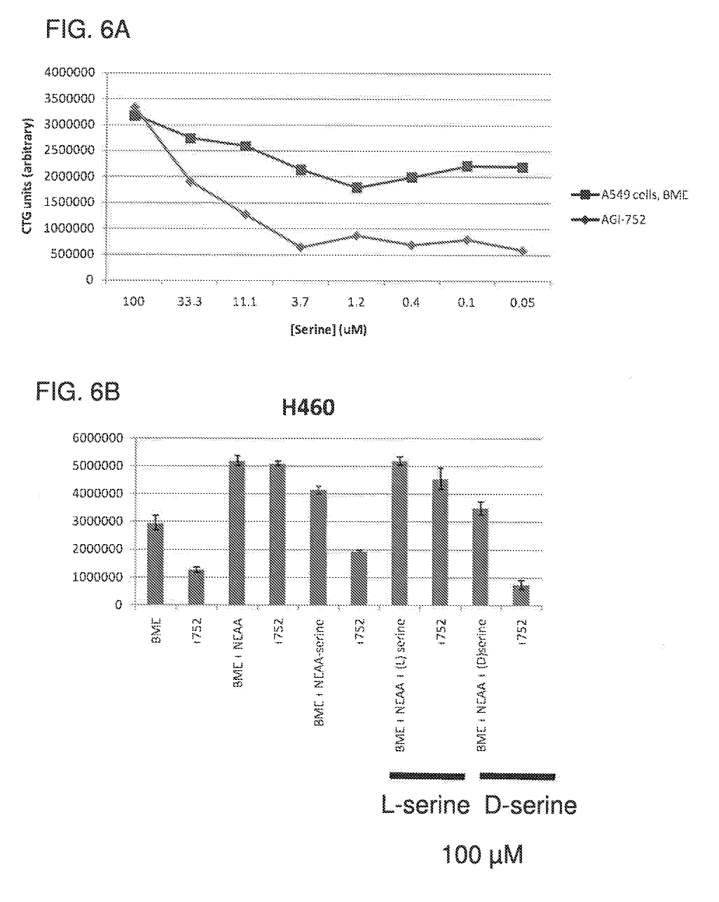

Serine rescued the toxic effect of AGI-752 in a dose-dependent manner (FIG. 6A). Cells were grown in BME, and serine was added in a 3-fold dose titration starting at 100 μM (“1×”). D-serine, however, was not capable of rescuing the effect of the compound (FIG. 6B).

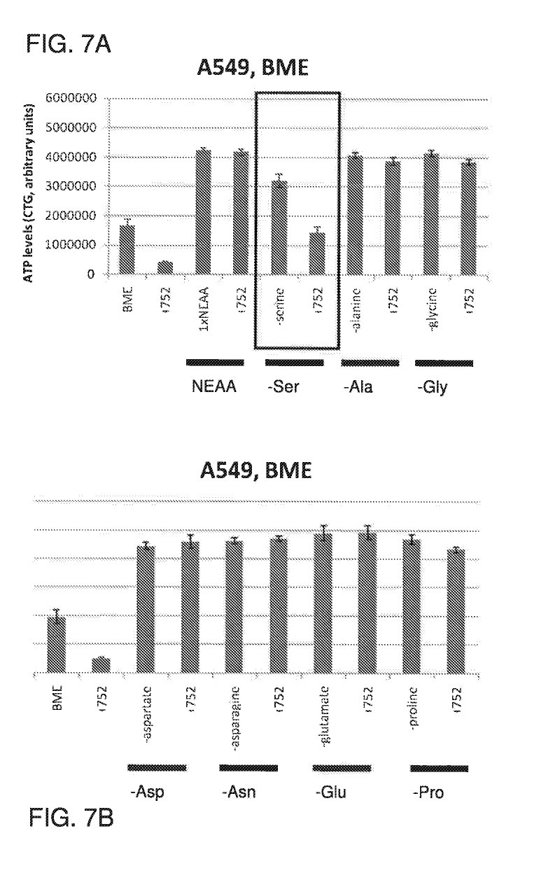

It was also found that serine deprivation is necessary for sensitivity of A549 cells to AGI-752 (FIGS. 7A and 7B). A549 cells were grown in BME media. NEAA (7 amino acids) or NEAA without individual amino acids (6 amino acids) sas added back to the media. The results indicated that dropping out serine preserves most of the sensitivity of AGI-752, while restoring most of the cell growth. Similar experiments performed with H460 cells yielded the same results.

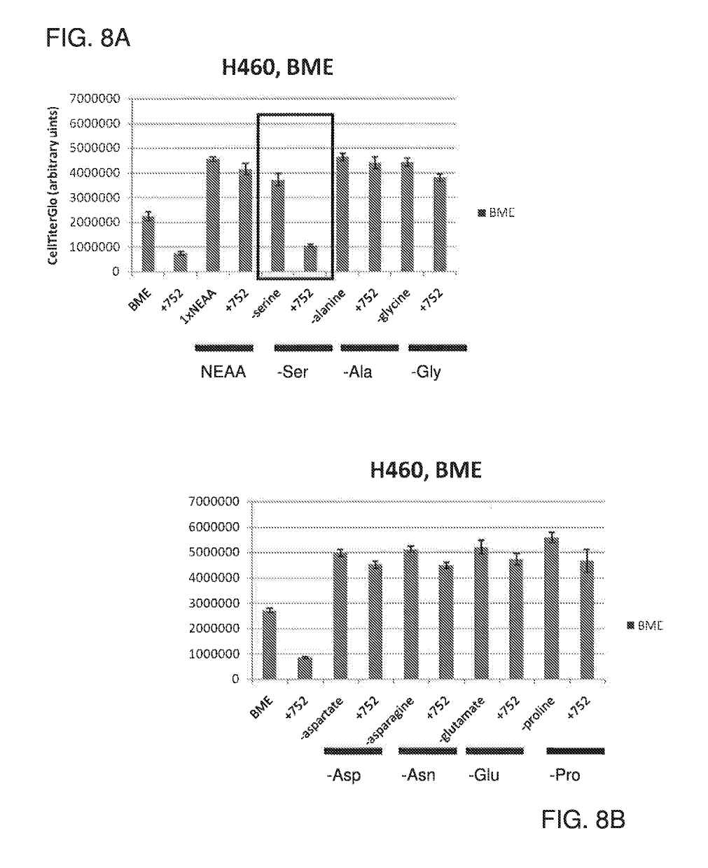

H460 cells were found to be fully sensitized to AGI-752 following serine deprivation in BME media. NEAA rescued the sensitivity except when serine was omitted from the media (FIGS. 8A and 8B).

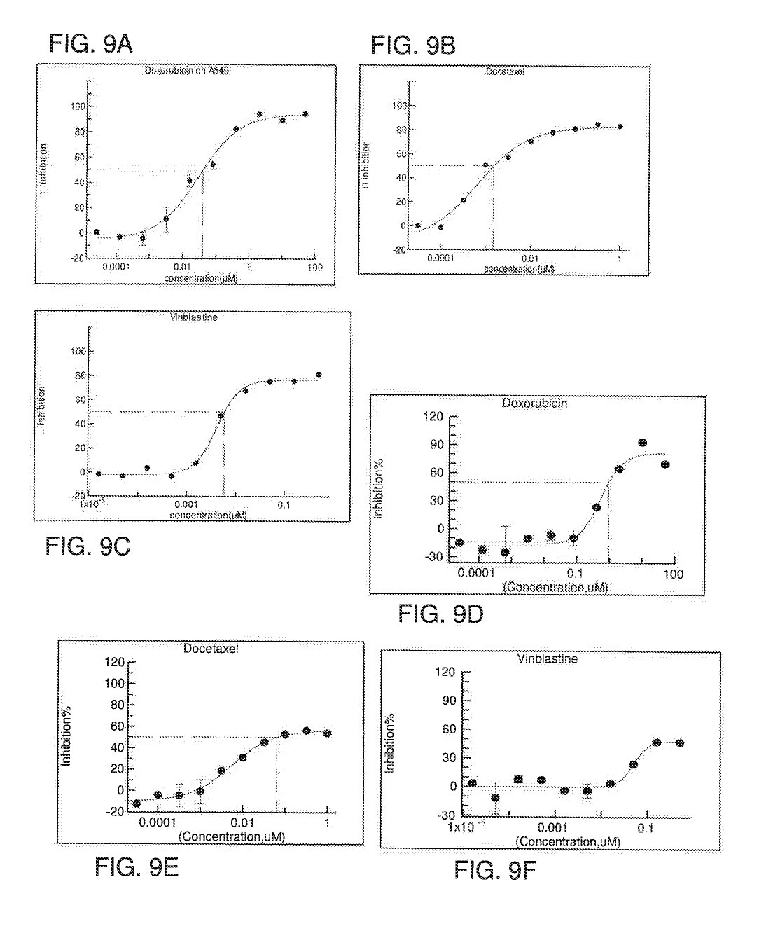

Cells grown in BME media are not generally more sensitive to cytotoxic agents (FIGS. 9A-9F). A549 cells were grown in RPMI (FIG. 9A, 9B or 9C) or in BME (FIGS. 9D, 9E, and 9F). Cells were treated with doxorubicin, docetaxel or vinblastine for 72 hours, and decreased potency was found to be consistent with slower cell proliferation in BME. Comparison of GI₅₀s side-by-side in regular RPMI versus BME medium indicated that the PKM2 activators of formula (V), compound A (an activator of PKM2), and compound B have a maximum of only about 60-70% growth inhibition in BME as compared to only 2-5% growth inhibition in RPMI. The effect of the PKM2 activators appears to by cytostatic (no induction of apoptosis).

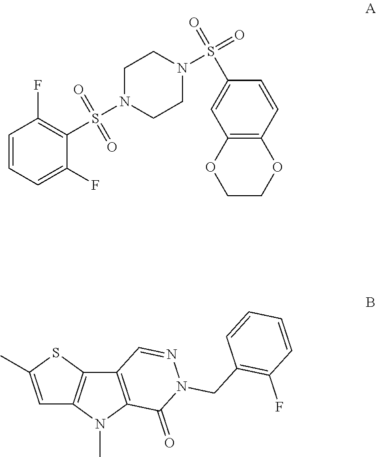 [SMILES: O=S(=O)(c1ccc2c(c1)OCCO2)N1CCN(S(=O)(=O)c2c(F)cccc2F)CC1 ; Cc1cc2c(s1)c1cnn(Cc3ccccc3F)c(=O)c1n2C]

There was no correlation observed between ex vivo AC50 and cell growth inhibition for A549 cells exposed to 81 different PKM2 activators over a 72 hr period, as measured by CTG (ATP content) in RPMI media with 10% FBS. This in contrast to the correlation observed between ex vivo AC50 and cell growth inhibition for A549 cells exposed to 100 different PKM2 activators over a 72 hr period, as measured by CTG in BME media. This data strongly suggests that the anti-proliferative effect in BME media is due to PKM2 activation.

Serine rescued the effect on cell proliferation in BME media of multiple PKM2 activator scaffolds. Cells were grown in BME±serine, and ±μM PKM2 activators.

To identify a genotype signature of PKM2 activators, cell lines were screened to identify lines with serine-dependent sensitivity to PKM2 activators. Cells were grown with BME-NEAA or BME-NEAA-serine amino acids, and cells were treated with the PKM2 activators. A full 11-point dose response was performed. 5 cell lines are identified as having sensitivity to PKM2 activation in BME and NEAA minus serine media. At least three of the five (A549, NCI-H460, and Colo-205) have low mRNA levels of phosphoserine phosphatase. The five cell lines are described in the below table.

| Cell Line | Cancer Type | Mutations |
|---|---|---|
| A549 | Non-small cell lung carcinoma | CDKN2A, KRAS, STK11 |
| NCI-H460 | Lung large cell carcinoma | CDKN2A, KRAS, |
| | | PI3KCA, STK11 |
| Colo-205 | Colon carcinoma | APC, BRAF, SMAD4, TP53 |
| MiaPaca-2 | Pancreatic cancer | CDKN2A, KRAS, TP53 |
| THP-1 | Acute monocytic leukemia | CDKN2A, NRAS, TP53 |

All publications, patents, and patent applications mentioned in this specification are herein incorporated by reference to the same extent as if each independent publication or patent application was specifically and individually indicated to be incorporated by reference.

A number of embodiments have been described. Nevertheless, it will be understood that various modifications may be made without departing from the spirit and scope of the invention.

We claim:

1. A method of diagnosing and treating a patient who has a cell proliferative-related disorder that is a candidate for treatment with a compound that activates PKM2, the method comprising:
diagnosing the patient as a candidate for treatment with a compound that activates PKM2 based on abnormally low levels of phosphoserine phosphatase mRNA or protein, or abnormally low levels of phosphoserine phosphatase activity, determined to be present in a sample from the subject, as compared to a reference standard, thereby measuring serine levels in the sample, to thereby diagnose the patient; and
administering an effective amount of a compound that activates PKM2.

2. The method of claim 1, wherein the compound is a small molecule.

3. The method of claim 1, wherein the sample comprises a serum sample or a tissue sample.

4. The method of claim 3, wherein the tissue sample is a sample from a tumor sample or from a tissue suspected of having cancerous cells.

5. The method of claim 1, wherein the cell proliferative-related disorder is cancer.

6. The method of claim 5, wherein the cancer is selected from the group consisting of a leukemia, polycythemia vera, lymphoma, Waldenstrom's macroglobulinemia, multiple myeloma, heavy chain disease, and a solid tumor.

7. The method of claim 6, wherein the leukemia is selected from the group consisting of acute leukemia, acute lymphocytic leukemia, acute myelocytic leukemia, acute myeloblasts leukemia, acute promyelocyte leukemia, acute myelomonocytic leukemia, acute monocytic leukemia, acute erythroleukemia, chronic leukemia, chronic myelocytic leukemia, and chronic lymphocytic leukemia.

8. The method of claim 6, wherein the lymphoma is Hodgkin's disease or non-Hodgkin's disease.

9. The method of claim 6, wherein the solid tumor is a sarcoma or a carcinoma.

10. The method of claim 9, wherein the sarcoma or carcinoma is selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma.

11. The method of claim 1, wherein diagnosing the patient as a candidate for treatment with a compound that activates PKM2 is based on abnormally low levels of phosphoserine phosphatase mRNA or protein.

12. The method of claim 1, wherein diagnosing the patient as a candidate for treatment with a compound that activates PKM2 is based on abnormally low levels of phosphoserine phosphatase activity.

13. The method of claim 1, comprising administering a second therapeutic agent that lowers the serine levels.

14. The method of claim 13, wherein the second therapeutic agent is an inhibitor of serine metabolism or disrupts a component of the phosphoserine pathway.
